(19) United States
(12) Patent Application Publication
(10) Pub. No.: US 2026/0035665 A1
(43) Pub. Date: Feb. 5, 2026

(54) SYSTEMS FOR CELL PROGRAMMING TOWARDS HEMATOPOIETIC LINEAGE AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US); Nikolas George Koutis BALANIS, Chicago, IL (US); Stephen Oren ARNOVITZ, Chicago, IL (US); Hugh HARDING, Chicago, IL (US); Renee Francesca Geraldine DE POOTER, Chicago, IL (US)

(21) Appl. No.: 19/298,640

(22) Filed: Aug. 13, 2025

Related U.S. Application Data
(63) Continuation of application No. PCT/US2024/016003, filed on Feb. 15, 2024.
(60) Provisional application No. 63/446,228, filed on Feb. 16, 2023.
(60) Provisional application No. 63/620,361, filed on Jan. 12, 2024.

(51) Int. Cl. C12N 5/0789; C12N 9/22; C12N 15/113; C12N 15/63

(52) U.S. Cl. CPC C12N 5/0647; C12N 9/222; C12N 15/113; C12N 15/635; C12N 2310/20; C12N 2506/03

(57) ABSTRACT

Provided herein are systems of modulating gene expression, methods of use thereof, and cells engineered thereof for the purpose of differentiating cells, for example hematopoietic cells.

### CROSS-REFERENCE

This application is a continuation of International Application No. PCT/US2024/016003, filed on Feb. 15, 2024 and claims the benefit of U.S. Provisional Patent Application No. 63/446,228, filed on Feb. 16, 2023, and U.S. Provisional Patent Application No. 63/620,361, filed on Jan. 12, 2024, each of which is incorporated herein by reference in its entirety.

### SEQUENCE LISTING

The instant application contains a Sequence Listing which has been submitted electronically in XML file format and is hereby incorporated by reference in its entirety. Said XML copy, created on Aug. 12, 2025, is named 61684-712_301_SL.xml and is 440,405 bytes in size.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell. In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species.

### SUMMARY

The present disclosure provides methods for converting a plurality of stem cells (first plurality) to a plurality of hematopoietic lineage cells (second plurality), the method comprising: culturing ex vivo the first plurality in a medium that is substantially free of one or more exogenous factors selected from the group consisting of thrombopoietin (TPO), exogenous FLT-3 Ligand (FLT3L), and interleukin (IL), wherein, within about 14 days following the culturing, a conversion rate from the first plurality to the second plurality is at least about 5%, wherein the second plurality is CD45+.

The present disclosure provides methods for converting a plurality of stem cells (first plurality) to a plurality of hematopoietic lineage cells (second plurality), the method comprising: contacting the first plurality with a heterologous genetic circuit comprising a plurality of gate units, wherein, upon activation of the heterologous genetic circuit, the plurality of gate units operates in concert to modulate expression levels of a plurality of distinct target genes in a sequential manner, wherein the plurality of gate units comprises: a first gate unit that is activatable, upon the activation of the heterologous genetic circuit, modulate expression of a target gene; and a second gate unit that is activatable, upon the activation of the first gate unit, to modulate expression of an additional target gene that is different from the target gene, such that modulation of the expression of the additional target gene is induced subsequent to modulation of the expression of the target gene, wherein the target gene comprises one or more members selected from the group consisting of TBXT, TBX6, MIXL1, and a combination thereof; and the additional target gene comprises one or more members selected from the group consisting of ETS1, ETV2, GATA2, SCL, LMO2, and a combination thereof, and wherein, upon the contacting, the plurality of gate units operates in concert to effect the conversion of the first plurality to the second plurality.

The present disclosure provides methods for converting a plurality of stem cells (first plurality) to a plurality of hematopoietic lineage cells (second plurality), the method comprising: contacting the first plurality with a heterologous genetic circuit comprising a plurality of gate units, wherein, upon activation of the heterologous genetic circuit, the plurality of gate units operates in concert to modulate expression levels of a plurality of distinct target genes in a sequential manner, wherein the plurality of gate units comprises: a first gate unit that is activatable, upon the activation of the heterologous genetic circuit, modulate expression of a target gene; and a second gate unit that is activatable, upon the activation of the first gate unit, to modulate expression of an additional target gene that is different from the target gene, such that modulation of the expression of the additional target gene is induced subsequent to modulation of the expression of the target gene, wherein the heterologous genetic circuit is programmed such that: (x) the target gene comprises one or more members selected from the group consisting of TBXT, TBX6, MIXL1, and a combination thereof; and the additional target gene comprises one or more members selected from the group consisting of HOXA5, HOXA9, ERG, LCOR, RUNX1, and a combination thereof; and/or (y) the target gene comprises one or more members selected from the group consisting of ETS1, ETV2, GATA2, SCL, LMO2, and a combination thereof; and the additional target gene comprises one or more members selected from the group consisting of HOXA5, HOXA9, ERG, LCOR, RUNX1, and a combination thereof, wherein, upon the contacting, the plurality of gate units operates in concert to effect the conversion of the first plurality to the second plurality.

The present disclosure provides methods for converting a plurality of stem cells (first plurality) to a plurality of hematopoietic lineage cells (second plurality), the method comprising: contacting a target gene in the first plurality by a heterologous gene regulating moiety to modulate expression and/or activity level of the target gene, wherein the target gene encodes a erythroblast transformation specific (ETS) transcription factor, wherein the contacting effects conversion of the first plurality to the second plurality.

The present disclosure provides methods for directing a plurality of stem cells towards differentiation, the method comprising: contacting the plurality of stem cells with a heterologous genetic circuit comprising a plurality of gate units, wherein, upon activation of the heterologous genetic circuit, the plurality of gate units operates in concert to induce a plurality of distinct modulations of a target gene in the plurality of stem cells in a sequential manner, each of the plurality of distinct modulations being necessary but individually insufficient to effect the directing of the plurality of stem cells towards differentiation, wherein the plurality of gate units comprises: (i) a first gate unit that is activatable, upon the activation of the heterologous genetic circuit, to induce a first distinct modulation of the plurality of distinct modulations; and (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell, wherein the target gene encodes an erythroblast transformation specific (ETS) transcription factor, and wherein, upon the contacting, the plurality of gate units operates in concert to effect the directing the plurality of stem cells towards differentiation.

The present disclosure provides systems for converting a plurality of stem cells (first plurality) to a plurality of hematopoietic lineage cells (second plurality), the system comprising: a medium for ex vivo culture of the first plurality, wherein the medium is substantially free of one or more exogenous factors selected from the group consisting of thrombopoietin (TPO), exogenous FLT-3 Ligand (FLT3L), and interleukin (IL), wherein, within about 14 days following ex vivo culturing of the first plurality in the medium, a conversion rate from the first plurality to the second plurality is at least about 5%, wherein the second plurality is CD45+.

The present disclosure provides systems for converting a plurality of stem cells (first plurality) to a plurality of hematopoietic lineage cells (second plurality), the system comprising: a heterologous genetic circuit comprising a plurality of gate units, wherein, upon activation of the heterologous genetic circuit, the plurality of gate units configured to operate in concert to modulate expression levels of a plurality of distinct target genes in a sequential manner, wherein the plurality of gate units comprises: a first gate unit that is activatable, upon the activation of the heterologous genetic circuit, modulate expression of a target gene; and a second gate unit that is activatable, upon the activation of the first gate unit, to modulate expression of an additional target gene that is different from the target gene, such that modulation of the expression of the additional target gene is induced subsequent to modulation of the expression of the target gene, wherein the target gene comprises one or more members selected from the group consisting of TBXT, TBX6, MIXL1, and a combination thereof; and the additional target gene comprises one or more members selected from the group consisting of ETS1, ETV2, GATA2, SCL, LMO2, and a combination thereof, and wherein, upon contacting of the first plurality by the heterologous genetic circuit, the plurality of gate units operates in concert to effect the conversion of the first plurality to the second plurality.

The present disclosure provides systems for converting a plurality of stem cells (first plurality) to a plurality of hematopoietic lineage cells (second plurality), the system comprising: a heterologous genetic circuit comprising a plurality of gate units, wherein, upon activation of the heterologous genetic circuit, the plurality of gate units configured to operate in concert to modulate expression levels of a plurality of distinct target genes in a sequential manner, wherein the plurality of gate units comprises: a first gate unit that is activatable, upon the activation of the heterologous genetic circuit, modulate expression of a target gene; and a second gate unit that is activatable, upon the activation of the first gate unit, to modulate expression of an additional target gene that is different from the target gene, such that modulation of the expression of the additional target gene is induced subsequent to modulation of the expression of the target gene, wherein the heterologous genetic circuit is programmed such that: (x) the target gene comprises one or more members selected from the group consisting of TBXT, TBX6, MIXL1, and a combination thereof; and the additional target gene comprises one or more members selected from the group consisting of HOXA5, HOXA9, ERG, LCOR, RUNX1, and a combination thereof; and/or (y) the target gene comprises one or more members selected from the group consisting of ETS1, ETV2, GATA2, SCL, LMO2, and a combination thereof; and the additional target gene comprises one or more members selected from the group consisting of HOXA5, HOXA9, ERG, LCOR, RUNX1, and a combination thereof, wherein, upon contacting of the first plurality by the heterologous genetic circuit, the plurality of gate units operates in concert to effect the conversion of the first plurality to the second plurality.

The present disclosure provides systems for converting a plurality of stem cells (first plurality) to a plurality of hematopoietic lineage cells (second plurality), the system comprising: a heterologous gene regulating moiety exhibiting specific binding to a target gene in the first plurality to modulate expression and/or activity level of the target gene, wherein the target gene encodes a erythroblast transformation specific (ETS) transcription factor, wherein contacting of the target gene by the heterologous gene regulating moiety effects conversion of the first plurality to the second plurality.

The present disclosure provides systems for directing a plurality of stem cells towards differentiation, the system comprising: a heterologous genetic circuit comprising a plurality of gate units, wherein, upon activation of the heterologous genetic circuit, the plurality of gate units operates in concert to induce a plurality of distinct modulations of a target gene in the plurality of stem cells in a sequential manner, each of the plurality of distinct modulations being necessary but individually insufficient to effect the directing of the plurality of stem cells towards differentiation, wherein the plurality of gate units comprises: (i) a first gate unit that is activatable, upon the activation of the heterologous genetic circuit, to induce a first distinct modulation of the plurality of distinct modulations; and (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell, wherein the target gene encodes an erythroblast transformation specific (ETS) transcription factor, and wherein, upon contacting of the plurality of stem cells by the heterologous genetic circuit, the plurality of gate units operates in concert to effect the directing the plurality of stem cells towards differentiation.

The present disclosure provides engineered cells comprising any one of the systems disclosed herein. The present disclosure provides compositions comprising any one of the systems and/or any one of the engineered cells disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic of the heterologous genetic circuit. An activating moiety initiates the circuit and can activate a gate unit. A gate unit is comprised of a gate moiety and/or a gene regulating moiety.

FIG. 2 depicts exemplary heterologous genetic circuits.

FIG. 3 shows scatter plots (i.e., a volcano plots) at days 3, 5, and 9 to identify one or more heterologous genetic circuits that differentiated induced pluripotent stem cells (iPSCs) into hematopoietic progenitor cells.

FIGS. 4A-4D show examples of hematopoietic progenitor cell marker analysis data used to generate the scatter plots of FIG. 3. FIG. 4A shows results for cell marker CD34⁺. FIG. 4B shows results for cell marker CD43⁺. FIG. 4C shows results for cell marker CD45⁺. FIG. 4D shows results for the combination of cell markers CD34⁺/CD43⁻/CD45⁺. The left and right dots represent replicate samples for each condition. The center dot represents mean value.

FIGS. 5A-5D show that the heterologous genetic circuits generate substantially more hematopoietic progenitor cells after five days in culture as compared to directed differentiation. FIG. 5A shows cell marker results for CD34⁺ and CD45⁺ markers. FIG. 5B shows additional cell marker results for CD34⁺, CD43⁺, and CD45⁺ markers. FIG. 5C shows a summary of the data of FIG. 5B. FIG. 5D shows a summary of the results from a repeat experiment.

FIG. 6 depicts the developmental cell lineage from a pluripotent stem cell (iPSC) to a hematopoietic progenitor cell (HPC). Cell surface marker (e.g. KDR, CD34, CD43, CD45) expression indicative of cell states are shown above cells. Genes encoding transcription factors are noted in the arrows indicating relative sequential order of activity for lineage progression. Exemplary heterologous genetic circuits utilizing such genes are depicted in Appendix Table.

FIG. 7 depicts design concepts for how either three sets of TFs or four sets of TFs are distributed in a 10-step cascade. Spacing paradigms were designed to accommodate future addition of later steps, temporally distribute gene activation steps, and maximize efficiency of heterologous gene circuit activation of endogenous genes.

FIGS. 8A-8D show the effect of heterologous gene circuits (CGO ###; described in Table 4) and control conditions (described in Table 4) on the differentiation of iPSC 7 days after introduction. Flow cytometry data show effects on the cell surface marker (KDR/CD309) indicative of early stages in lineage differentiation. FIG. 8D shows a summary of the percent of KDR+ cells determined from flow cytometry data.

FIGS. 9A-9D show the effect of heterologous gene circuits (CGO ###; described in Table 4) and control conditions (described in Table 4) on the differentiation of iPSC 7 days after introduction. Flow cytometry data show effects on the cell surface marker (CD34) indicative of intermediate stages in lineage differentiation. FIG. 9D shows a summary of the percent of KDR− CD34+ (hemogenic endothelium) cells determined from flow cytometry data.

FIGS. 10A-10D show the effect of heterologous gene circuits (CGO ###; described in Table 4) and control conditions (described in Table 4) on the differentiation of iPSC 7 days after introduction. Flow cytometry data show effects on cell surface markers (KDR/CD309; CD34) indicative of intermediate stages in lineage differentiation. FIG. 10D shows a summary of the percent of KDR− CD34+ (hemogenic endothelium) cells determined from flow cytometry data.

FIGS. 11A-11D show the effect of heterologous gene circuits (CGO ###; described in Table 4) and control conditions (described in Table 4) on the differentiation of iPSC 7 days after introduction. Flow cytometry data show effects on cell surface markers (CD43; CD34) indicative of HPC stage. FIG. 11D shows a summary of the percent of double positive (CD43+CD34+) cells determined from flow cytometry data.

FIGS. 12A-12D show the effect of heterologous gene circuits (CGO ###; described in Table 4) and control conditions (described in Table 4) on the differentiation of iPSC 7 days after introduction. Flow cytometry data show effects on cell surface markers (CD45; CD34) indicative of HPC stage. FIG. 12D shows a summary of the percent of double positive (CD45+CD34+) cells determined from flow cytometry data.

FIGS. 13A-13D show the yield in total cell numbers 6 days after seeding 3000 cells from 7-day differentiation procedures (analyzed in FIGS. 9A-11D; described in Table 4) into a DLL4-containing and growth factor rich expansion media. Pan-hematopoietic (CD45 total; FIG. 13A), HPC (CD34+CD45+; FIG. 13B), Lineage committed (CD34− CD45+; FIG. 13C), and Lymphoid (CD7; FIG. 13D) cell types are shown.

FIGS. 14A-14B show the effect of heterologous gene circuits (CGO ###; described in Table 4) compared to a benchmark protocol for CD34+CD45+CD43+ cells. FIG. 14A shows CGO178 with iPSC and transient plasmids at day 7. FIG. 14B shows the benchmark protocol with transgenic iPSC line at day 15.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

### Definitions

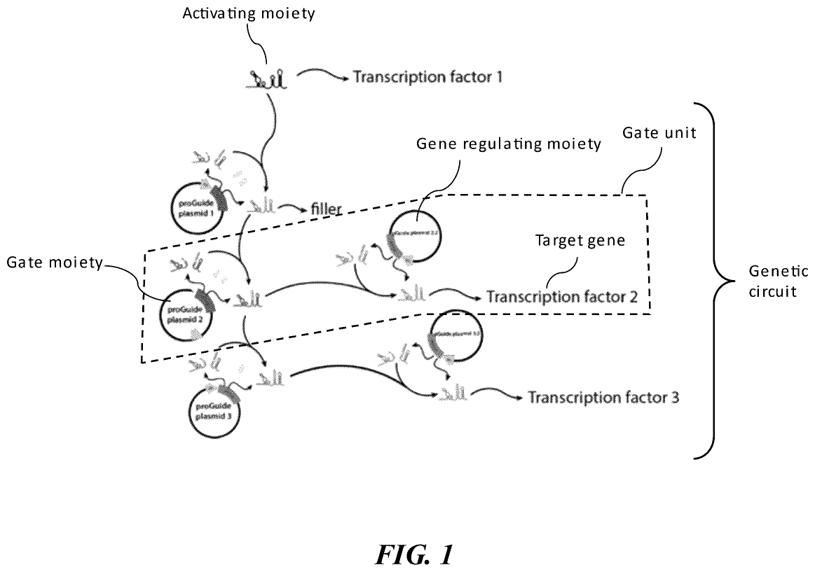

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 1).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units is activatable by an activating moiety (e.g., a heterologous input to the cell) to activate other gate units of the plurality of gate units (e.g., simultaneously at once, sequentially in a cascading manner, etc.) (FIG. 1). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 1). The terms “heterologous genetic circuit,” “HGC,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription/translation regulatory region, or series of regions, which the genetic switch acts on. The input for a gate unit can be an activating moiety and/or another gate unit. The output for a gate unit can be to activate another gate unit, to de-activate another gate unit, to affect a target gene, and/or a combination of any of the above. For example, a gate unit can be comprised of a plurality of gate moieties and/or a plurality of gene regulating moieties (FIG. 1).

The term “activating moiety,” as referred to herein, generally refers to a moiety that can activate plurality of genetic circuits and/or a plurality of gate units. An activating moiety can be a heterologous input to a cell. In some cases, activating moieties can include, but are not limited to, a guide nucleic acid molecule (e.g., a gRNA) or other nucleic acid, polypeptides, polynucleotides, small molecules, light, or a combination thereof. For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties. The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively, or in addition to, a gate moiety can activate and/or deactivate another gate unit of the genetic circuit (FIG. 1). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 1). For example, a gene editing moiety can regulate expression of a gene by editing a nucleic acid sequence (e.g., CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g., CRISPR-Cas-inspired RNA targeting systems). Alternatively, a gene editing moiety can repress translation of a gene (e.g., Cas13).

Alternatively, or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g., Cas12).

In some cases, a plasmid can encode a non-functional form of a gene editing moiety. The plasmid can be activated to express a functional form of the gene editing moiety, e.g., via activation of a functional gate moiety. For example, the gene editing moiety can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited to allow expression of a functional form of the gene editing moiety (e.g., a functional form of the guide nucleic acid molecule with specific binding to the target gene of the cell), to permit modulation of the target gene in the cell.

Accordingly, a gene regulating moiety can comprise a nucleic acid molecule (e.g., a guide nucleic acid molecule that forms a complex with an endonuclease, such as a Cas protein). Alternatively, or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

A gene regulating moiety can be a transcriptional modulator system (e.g., a gene repressor complex or a gene activator complex). For example, a gene regulating moiety can be a gene repressor complex comprising a dCas protein operatively coupled to (e.g., fused to) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASC1, JMJD2D, JARID1A, RBP2, JARID1B/PLU-1, JARIDIC/SMCX, JARIDID/SMCY, HDAC1, HDAC2, HDAC3, HDAC8, HDAC4, HDAC5, HDAC7, HDAC9, SIRT1, SIRT2, HDAC11, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SET1A, SET1B, MLL1, MLL2, MLL3, MLL4, MLL5, ASH1, SYMD2, NSD1, JHDM2a, JHDM2b, UTX, JMJD3, GCN5, PCAF, CBP, p300, TAF1, TIP60/PLIP, MOZ/MYST3, MORF/MYST4, SRCI, ACTR, P160, CLOCK, TET1CD, TET1, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TET1CD), TET1, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g., altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g., deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [RI 10]dCTP, [R6G]dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “reprogramming,” “dedifferentiation,” “increasing cell potency,” or “increasing developmental potency,” as used interchangeable herein, generally refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “pluripotent” generally refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (i.e., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., natural killer (NK) cells. In some cases, iPSCs can be engineered to differentiate first into tissue-specific stem cells (e.g., hematopoietic stem cells (HSCs) or hematopoietic progenitor cells (HPCs)), which can be further induced to differentiate into committed cells (e.g., NK cells).

The term “embryonic stem cell” (ESCs) generally refers to cells derived from the naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiate directly into committed cells (e.g., NK cells). In some cases, ESCs can be engineered to differentiate first into tissue-specific stem cells (e.g., HSCs), which can be further induced to differentiate into committed cells (e.g., NK cells).

The term “isolated stem cells” generally refers to any type of stem cells disclosed herein (e.g., ESCs, HSCs, mesenchymal stem cells (MSCs), etc.) that are isolated from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The term “hematopoietic lineage” when applied to a cell or a population generally refers to a cell derived (e.g., differentiated) from a mesoderm cell (e.g., obtained from pluripotent stem cells), and includes, for example, hemogenic endothelium (HE), pre-hematopoietic stem cell (HSC), HSC, and hematopoietic progenitor cell (e.g., multipotent progenitor cell, lymphoid progenitor cell, early thymic progenitor cell, pre-T cell progenitor cell, pre-NK progenitor cell, T progenitor cell, NK progenitor cell, myeloid progenitor cell, etc.). In some cases, hematopoietic progenitor cells or HSCs can be characterized as being CD34+ (e.g., as compared to a pluripotent cell). In some cases, hematopoietic progenitor cells or HSCs cells can be characterized as being CD34+CD45+ or CD34+CD43−CD45+ (e.g., as compared to a pluripotent cell).

The term “hematopoietic stem and progenitor cells,” “hematopoietic stem cells,” “hematopoietic progenitor cells,” or “hematopoietic precursor cells,” as used interchangeably herein, generally refers to cells which are committed to a hematopoietic lineage but are capable of further hematopoietic differentiation (e.g., into NK cells) and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). In some cases, HSCs can be CD34+ hematopoietic cells capable of giving rise to both mature myeloid and lymphoid cell types including T cells, NK cells and B cells.

The term “immune cell” generally refers to a differentiated hematopoietic cell. Non-limiting examples of an immune cell can include an NK cell, a T cell, a monocyte, an innate lymphocyte, a tumor-infiltrating lymphocyte, a macrophage, a granulocyte, etc.

In some cases, the term “proGuide” as generally used herein may refer to such vector (e.g., a plasmid) that encodes the activatable gNA. The proGuide can be an example of a gate moiety. The proGuide can be an example of a gene regulating moiety.

In some embodiments, a proGuide as provided herein can encode an activatable guide nucleic acid molecule, e.g., having the inactivation polynucleotide sequence (e.g., one or more polyX sequences, such as one or more polyT sequences). In some cases, a portion of the proGuide encoding the activatable guide nucleic acid molecule can comprise various regions that are sequentially linked (e.g., from 5′ to 3′), comprising upstream stem (e.g., an upstream cut site), a poly T unit (or “proUnit” or “proGuide Unit” as used interchangeably herein), and a downstream stem (e.g., a downstream cut site). The upstream stem and the downstream stem may correspond to the “stem region” polynucleotide sequences that are at least partially complementary to each other.

### Overview

Biological programming, such as cellular programming, allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control a cascade of a plurality of desired expression and/or activity profiles of a plurality of genes in the cell. To allow for better control of specific cellular outcomes, genetic circuits can be multiplexed to create positive feedback and/or negative feedback systems.

Although CRISPR/Cas systems are widely used for gene editing, Cas is essentially a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this simple barcoding, often using exogenous fluorophores, doesn't allow for the multiplexed regulation of endogenous genes to effect cell differentiation.

Thus, there remains an unmet need for an activatable, multiplexed CRISPR/Cas system and use of the same to edit a target polynucleotide (e.g., a genome of a cell, in particular a eukaryotic cell), using cascades of gRNAs to form genetic circuits which include feedback loops in order to single-handedly affect gene regulation and, in turn, cell-fate determination. Given its improved multiplexing capabilities through the use of internal positive and/or negative feedback loops, the preprogrammed, activatable, and self-regulating gRNA cascade CRISPR/Cas system finds use, e.g., in gene therapy, genetic circuitry, and/or complex cell-fate determination and/or control.

The present disclosure provides systems and methods for engineering a CRISPR/Cas9 system, which includes a Cas endonuclease and an array of cognate single guide RNAs (sgRNA or gRNA) that harbor inactivation sequences in a non-essential region and are activatable, to allow for modulation and modification of that system by the use of positive and negative feedback loops. The present disclosure also provides for an engineered cell that can contain any of the above-mentioned systems or that can be capable of performing any of the above-mentioned methods.

The differentiation of iPSC into hematopoietic progenitor cells (HPC) has been made difficult by several barriers with various etiologies. Attempts at generating HPC using extracellular control mechanisms (culture conditions, growth factors, small molecules, etc) have had limited success on small scales, but are prone to reproducibility problems and batch to batch variability in efficiency and potency. Cells generated from these approaches typically display restricted lineage potential, incapable of contributing to critical lymphoid cell lineages. Consistent with other iPSC differentiation procedures based on controlling cellular microenvironments, these approaches are also difficult to scale up to levels of production and manufacturing required for therapeutic products.

Previous work showed that using the activity of TF gene products can drive iPSC to an HPC state with capabilities of making lymphoid, myeloid, and erythroid cell lineages. The expression of a cohort of 7 TFs was achieved by lentiviral insertion of cDNA expression transgenes into the genome of human pluripotent stem cells. The resulting cell line harbored multiple gene insertions, posing a barrier for use in humans due to significant safety concerns. Thus, while the uncontrolled genome modifications from lentiviral insertions would prevent use in humans, cells harboring the TF transgene insertions display improved HPC stem cell properties and contribution to lymphoid lineages compared to approaches that used only manipulation of extracellular conditions.

The approach described here uses non-integrating plasmid DNAs to deliver genetic instructions to iPSC. The activation of TF genes provides cells with capabilities as HPC that have not been easily attainable using external control approaches. The plasmid DNAs do not require genomic integration for activation of endogenous genes, thus obviating the safety concerns caused by multiple lentiviral transgene insertions. The genetic instructions also have the benefit of being sequentially delimited for improving the transition from one cell state to the next by the timely activation of stage-specific TF gene expression.

### Systems and Methods for Engineering Genetic Circuits, Cells Comprising Thereof, and Methods of Use Thereof

Various aspects of the present disclosure provide systems for inducing a desired expression and/or activity level (or profile thereof) of one or more target genes in a cell. Various aspects of the present disclosure provide methods for inducing a desired expression and/or activity level (or profile thereof) of one or more target genes in a cell.

In an aspect, the present disclosure provides for a system that induces a desired expression and/or activity profile of a target gene in a cell. The system can comprise a heterologous genetic circuit comprising a plurality of gate units. The plurality of gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s). The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). The plurality of gate units can be different (e.g., comprising different polynucleotide sequences).

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel paths), or a combination thereof.

A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g., cell proliferation assays, metabolic activity assays, cell killing assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

The outcome in the cell can comprise regulation of a target gene. The regulation of the target gene can comprise a plurality of distinct modulations of the target gene. The plurality of gate units can each induce one of the plurality of distinct modulations of the target gene, such that a collection of the distinct modulation in concert yields a final expression and/or activity profile of the target gene. At least two distinct modulations of the plurality of distinct modulations can both increase an expression and/or activity level of the target gene. At least two distinct modulations of the plurality of distinct modulations can both decrease an expression and/or activity level of the target gene. Alternatively, a first distinct modulation of the plurality of distinct modulations can increase an expression and/or activity level of the target gene, while a second distinct modulation of the plurality of distinct modulations can decrease the expression and/or activity level of the target gene. In such case, the first distinct modulation can occur prior to the second distinct modulation, or vice versa. Alternatively, a distinct modulation (e.g., a first and/or second modulation) of the plurality of distinct modulations can maintain an expression and/or activity level of the target gene at the level of expression and/or activity level prior to the modulation.

In some cases, each distinct modulation of the plurality of distinct modulations of the target gene, as disclosed herein, can be necessary but individually insufficient to effect the desired expression and/or activity profile of the target gene. Thus, the outcome in the cell (e.g., enhanced cell function, induced cell state, etc.) induced by the plurality of distinct modulations of the target gene may not be possible in absence of any one of the plurality of distinct modulations of the target gene. Alternatively, a degree or measure of the outcome in the cell induced by the plurality of distinct modulations of the target gene can be greater than a degree or measure of the outcome in a control cell that is induced by none, one or more, but not all of the plurality of distinct modulations of the target gene, and/or by all of the plurality of distinct modulation of the target genes occurring through a different sequential order of events.

A second gate unit can be activated by a first gate unit (e.g., directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.).

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively, or in addition to, inactivation can be achieved through the use of a transcriptional modulator system (e.g., a transcriptional repressor). An endonuclease-transcriptional modulator system (e.g., a Cas-repressor) can be used to achieve polynucleotide cleavage (e.g., for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively, or in addition to, the endonuclease-transcriptional modulator system (e.g., a Cas-repressor) can be used to modulate target gene expression.

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g., a gate moiety and/or a gene regulating moiety of the first gate unit) that is responsible for inducing the first distinct modulation of the target gene.

The plurality of gate units can be preconfigured such that one of the plurality of gate units is activatable, upon the activation of the heterologous genetic circuit, to modulate expression and/or activity level of an additional target gene. The additional target gene can be different from the target gene that is modulated by the plurality of distinct modulations as provided herein. For example, the target gene can comprise an erythroblast transformation specific (ETS) transcription factor, and the additional target gene can comprise a T-box transcription factor (TBX), a homeobox protein, a GATA, and/or a basic helix-loop-helix transcription factor (bHLH). In some cases, the first gate unit can be configured to be activated to modulate the expression and/or activity level of the additional target gene. For example, the first gate unit can comprise a plurality of activatable gene regulating moieties for modulating expression and/or activity levels of the target gene and the additional target gene, respectively. In some cases, the second gate unit can be configured to be activated to modulate the expression and/or activity level of the additional target gene. For example, the second gate unit can comprise a plurality of activatable gene regulating moieties for modulating expression and/or activity levels of the target gene and the additional target gene, respectively. In some cases, the plurality of gate units can comprise at least one additional gate unit configured to be activated to modulate the expression and/or activity level of the additional target gene. For example, the heterologous genetic circuit can be configured such that the at least one additional gate unit is activated prior to activation of the first gate unit, between activation of the first gate unit and the second gate unit, and/or subsequent to activation of the second gate unit.

In some cases, a first gate unit modulates a first target gene. Alternatively, or in addition to, a first gate unit can also modulate a second gate unit. The modulation of the second gate unit can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or after the modulation of the first gate unit, as ascertained by rt-qPCR, Western blotting, or other methods.

In some cases, the second gate unit can modulate a second target gene. The modulation of the second target gene can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or more after the modulation of the first target gene, as ascertained by rt-qPCR, Western blotting, or other methods.

In some cases, modification of a target gene by a gate unit can inactivate a gene. For example, modification of a gene can stop expression and/or activity level of a target gene. Alternatively, modification of a gene can decrease the expression and/or activity level of a target gene. In some cases, modification of a gene can increase the expression and/or activity level of a target gene. Alternatively, modification of a gene can maintain the expression and/or activity level of a target gene.

The at least one additional gate unit can comprise at least or up to about 1 gate unit, at least or up to about 2 gate units, at least or up to about 3 gate units, at least or up to about 4 gate units, at least or up to about 5 gate units, at least or up to about 6 gate units, at least or up to about 7 gate units, at least or up to about 8 gate units, at least or up to about 9 gate units, at least or up to about 10 gate units, at least or up to about 11 gate units, at least or up to about 12 gate units, at least or up to about 13 gate units, at least or up to about 14 gate units, at least or up to about 15 gate units, at least or up to about 20 gate units, at least or up to about 30 gate units, at least or up to about 40 gate units, or at least or up to about 50 gate units. Each of the at least one additional gate unit can comprise at least or up to about 1 gene regulating moiety, at least or up to about 2 gene regulating moieties, at least or up to about 3 gene regulating moieties, at least or up to about 4 gene regulating moieties, at least or up to about 5 gene regulating moieties, at least or up to about 6 gene regulating moieties, at least or up to about 7 gene regulating moieties, at least or up to about 8 gene regulating moieties, at least or up to about 9 gene regulating moieties, at least or up to about 10 gene regulating moieties, at least or up to about 15 gene regulating moieties, at least or up to about 20 gene regulating moieties, at least or up to about 30 gene regulating moieties, at least or up to about 40 gene regulating moieties, or at least or up to about 50 gene regulating moieties. Each of the gene regulating moieties of a gate unit can be configured to bind to different target genes, to modulate expression and/or activity level of the different target genes, respectively. The heterologous genetic circuit as provided herein can be configured to modulate expression and/or activity level of one or more of the target genes as provided herein. In some cases, the heterologous genetic circuit can be configured to modulate expression and/or activity level of some but not all of the target genes as provided herein. For example, the heterologous genetic circuit can be preconfigured to modulate expression and/or activity level of a first set of one or more target genes (e.g., an erythroblast transformation specific (ETS) gene such as ETS1, ETV2, LMO2, etc.), but may not be preconfigured to modulate expression and/or activity of a second set of one or more target genes (e.g., TBXT, TBX6, MIXL1, etc.).

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel paths), or a combination thereof. In some embodiments, the plurality of gate units in series can operate in a forward cascade. In some embodiments, the forward manner can follow a numerically increasing step order (e.g., step 1 to step 2 to step 3 to step 4 to step 5, etc). In some embodiments, the plurality of gate units in series can operate in a reverse cascade. In some embodiments, the reverse cascade can follow a numerically decreasing step order (e.g., step 10 to step 9 to step 8 to step 7 to step 6, etc). In some embodiments, the plurality of gate units in series can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). In some embodiments, the plurality of gate units in series can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g., cell proliferation assays, metabolic activity assays, cell killing assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

The heterologous genetic circuit can comprise a plurality of gate units that are sequentially activated, e.g., activated in series one after another. The plurality of gate units can comprise a functional gate unit that is preconfigured such that it is activated to regulate (e.g., directly regulate) expression and/or epigenetic profile of a target gene (e.g., an endogenous targe gene). The plurality of gate units can further comprise one or more additional gate units that are preconfigured (i) to be activated prior to the functional gate unit and (ii) to effect a subsequent activation of the functional gate unit. In some cases, the one or more additional gate units can be preconfigured to be activated to regulate one or more additional target genes. Alternatively, the one or more additional gate units may not be preconfigured to regulate any target gene (e.g., any endogenous target gene) when activated. Such one or more additional gate units may instead serve to delay (e.g., in terms of time) activation of the functional gate unit during operation of the heterologous genetic circuit, thereby delaying the expression and/or epigenetic profile of the target gene of the functional gate unit, and thus the one or more additional gate units may be referred to as “blank” gate unit(s). The heterologous genetic circuit can comprise at least or up to about 1 blank gate unit, at least or up to about 2 blank gate units, at least or up to about 3 blank gate units, at least or up to about 4 blank gate units, at least or up to about 5 blank gate units, at least or up to about 6 blank gate units, at least or up to about 7 blank gate units, at least or up to about 8 blank gate units, at least or up to about 9 blank gate units, at least or up to about 10 blank gate units, at least or up to about 11 blank gate units, at least or up to about 12 blank gate units, at least or up to about 13 blank gate units, at least or up to about 14 blank gate units, at least or up to about 15 blank gate units, at least or up to about 16 blank gate units, at least or up to about 27 blank gate units, at least or up to about 18 blank gate units, at least or up to about 19 blank gate units, at least or up to about 20 blank gate units, at least or up to about 25 blank gate units, at least or up to about 30 blank gate units, at least or up to about 35 blank gate units, at least or up to about 40 blank gate units, at least or up to about 45 blank gate units, at least or up to about 50 blank gate units.

In some cases, use of the one or more blank gate units can delay activation of the functional gate unit (e.g., as ascertained by measurement of expression/epigenetic profile of the target gene, or as ascertained by measurement of expression of a functional variant or transcribed product of the functional gate unit) by at least or up to about 1 minute, at least or up to about 5 minutes, at least or up to about 10 minutes, at least or up to about 30 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 11 hours, at least or up to about 12 hours, at least or up to about 13 hours, at least or up to about 14 hours, at least or up to about 15 hours, at least or up to about 16 hours, at least or up to about 17 hours, at least or up to about 18 hours, at least or up to about 19 hours, at least or up to about 20 hours, at least or up to about 21 hours, at least or up to about 22 hours, at least or up to about 23 hours, at least or up to about 24 hours, at least or up to about 2 days, at least or up to about 3 days at least or up to about 4 days at least or up to about 5 days at least or up to about 6 days, or at least or up to about 7 days.

In some embodiments, the disclosure provided herein as Appendix A discloses solutions to barriers to therapeutic HPC production. In some embodiments, Appendix A discloses a multi-step cascade for HPC generation. In some embodiments, Appendix A discloses placing activation of transcription factor genes into a Cellgorithm library design. In some embodiments, Appendix A discloses a 10-step cascade for HPC generation. In some embodiments, the 10-step cascade can include blank steps and/or unused steps. In some embodiments, the blank steps and/or unused steps can space out factor activation, leave room for additional genes, maximize efficiency of Cellgorithm gene target induction, and/or modulate relative timing of each step. Appendix A discloses Cellgorithms producing a range of HPC expandability and yield of HPCs.

In some cases, a guide nucleic acid molecule (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least 1, at least 2, at least 3, at least 4, at least 5, at least 6, at least 7, at least 8, at least 9, at least 10, or more single-stranded breaks. In some cases, inactivation of a gene can be caused by at most 10, at most 9, at most 8, at most 7, at most 6, at most 5, at most 4, at most 3, at most 2, or at most 1 single-stranded breaks.

In some cases, modification of a polynucleotide sequence (e.g., as a component of a gate unit, such as a gate moiety) or a target gene can be caused by a double-stranded break wherein there is a discontinuity in both nucleotide strands. In some cases, inactivation of a gene can be caused by at least 1, at least 2, at least 3, at least 4, at least 5, at least 6, at least 7, at least 8, at least 9, at least 10, or more double-stranded breaks. In some cases, inactivation of polynucleotide sequence or a target gene can be caused by at most 10, at most 9, at most 8, at most 7, at most 6, at most 5, at most 4, at most 3, at most 2, or at most 1 double-stranded breaks.

In some cases, a gNA is at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides or more in length.

In some cases, the system and methods of the present disclosure can utilize at least two different endonucleases. A first endonuclease (e.g., a Cas protein that is not coupled to a transcriptional modulator) can be used in conjunction with a guide nucleic acid to induce a cleavage of a target polynucleotide sequence of a gate moiety or a gene regulating moiety plasmids, to activate and/or deactivate the gate moiety or the gene regulating moiety, respectively. In addition, a second endonuclease (e.g., a Cas protein that is coupled to a transcriptional modulator) can be used in conjunction with another guide nucleic acid to bind to a target gene (e.g., a target endogenous gene) of a cell, to modulate expression and/or activity of the target gene without generating a cleavage in the target gene.

In some cases, the systems and methods of the present disclosure can utilize a single endonuclease system (e.g., a Cas-repressor) to achieve both (i) polynucleotide cleavage (e.g., for activating/inactivating the gate moiety and/or the gene regulating moiety) and (ii) modulation of target gene expression. When using a single endonuclease-transcriptional modulator system, unique guide nucleic acid molecules (gNAs) of differing spacer sequence lengths can be used to determine whether the single endonuclease-transcriptional modulator system may (i) hybridize to the polynucleotide sequence to induce Cas-mediated nuclease activity of the polynucleotide sequence, or (ii) can hybridize to a target gene (e.g., genomic DNA) to modulate expression and/or activity level of the target gene via action of the transcriptional activator without mediating Cas nuclease activity, as desired by the individual heterologous genetic circuit. For example, use of gNAs of differing spacer sequence lengths that bind to different targets can allow for a second gate unit as provided herein to induce inactivation of a first gate unit that has been activated and/or induce a distinct modulation of a second target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g., a Cas or a Cas-transcriptional modulator fusion protein) at a target polynucleotide sequence. Alternatively, or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g., a Cas-activator system, a Cas-repressor system), without cleavage of the target gene.

In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein.

In some cases, modification of a polynucleotide sequence (e.g., as a component of a gate unit, such as a gate moiety) or a target gene can inactivate a gene. For example, modification of a polynucleotide sequence or a target gene can stop expression and/or activity level of the polynucleotide sequence or the target gene. Alternatively, modification of a polynucleotide sequence or a target gene can decrease the expression and/or activity level of the polynucleotide sequence of the target gene. In some cases, modification of a polynucleotide sequence or a target gene can increase the expression and/or activity level of the polynucleotide sequence or the target gene. Alternatively, modification of a polynucleotide sequence or a target gene can maintain the expression and/or activity level of the polynucleotide sequence or the target gene.

In some cases, modification of a polynucleotide sequence (e.g., as a component of a gate unit, such as a gate moiety) or a target gene can comprise decreasing the expression and/or activity level of the polynucleotide sequence or the target gene, respectively, by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polynucleotide sequence or a target gene can comprise decreasing the expression and/or activity level of the polynucleotide sequence or the target gene, respectively, by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polynucleotide sequence (e.g., as a component of a gate unit, such as a gate moiety) or a target gene can comprise increasing the expression and/or activity level of the polynucleotide sequence or the target gene, respectively, by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polynucleotide sequence or a target gene can comprise increasing the expression and/or activity level of the polynucleotide sequence or the target gene, respectively, by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polynucleotide sequence (e.g., as a component of a gate unit, such as a gate moiety) or a target gene can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a polynucleotide sequence or a target gene can comprise decreasing the expression and/or activity level of the polynucleotide sequence or the target gene, respectively, by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

In some cases, modification of a polynucleotide sequence (e.g., as a component of a gate unit, such as a gate moiety) or a target gene can comprise increasing the expression and/or activity level of the polynucleotide sequence or the target gene, respectively, by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a polynucleotide sequence or a target gene can comprise increasing the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

In some cases, (i) a change (e.g., enhancement or reduction) in the expression and/or activity level of the target gene upon the plurality of distinct modulations of the target gene in the sequential manner can be greater than (ii) any change in expression and/or activity level of the target gene upon only one or none of the plurality of distinct modulations, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 100%, at least or up to about 125%, at least or up to about 150%, at least or up to about 175%, at least or up to about 200%, at least or up to about 225%, at least or up to about 250%, at least or up to about 275%, or at least or up to about 300%.

In some cases, (i) a change (e.g., enhancement or reduction) in the expression and/or activity level of the target gene upon the plurality of distinct modulations of the target gene in the sequential manner can last for a longer duration than (ii) any change in expression and/or activity level of the target gene upon only one or none of the plurality of distinct modulations, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 100%, at least or up to about 125%, at least or up to about 150%, at least or up to about 175%, at least or up to about 200%, at least or up to about 225%, at least or up to about 250%, at least or up to about 275%, or at least or up to about 300%.

In some cases, (i) the expression and/or activity level of the target gene upon the plurality of distinct modulations of the target gene in the sequential manner (e.g., including the sequential disruption) can be less than (ii) expression and/or activity level of the target gene upon only one the first distinct modulation but not the second distinct modulation, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least up to or about 91%, at least up to or about 92%, at least up to or about 93%, at least up to or about 94%, at least up to or about 95%, at least up to or about 96%, at least up to or about 97%, at least up to or about 98%, at least up to or about 99%, or substantially about 100%.

In some cases, (i) the expression and/or activity level of the target gene upon the plurality of distinct modulations of the target gene in the sequential manner (e.g., including the sequential disruption) can last shorter than (ii) expression and/or activity level of the target gene upon only one the first distinct modulation but not the second distinct modulation, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least up to or about 91%, at least up to or about 92%, at least up to or about 93%, at least up to or about 94%, at least up to or about 95%, at least up to or about 96%, at least up to or about 97%, at least up to or about 98%, at least up to or about 99%, or substantially about 100%.

In some cases, the first gate unit and the second gate unit as provided here can be activated to induce a first distinct modulation and a subsequent second distinct modulation of a common gene (e.g., expression and/or activity profile of the common gene). The first gate unit and the second gate unit may be activated sequentially (e.g., at different time points), to effect affect the first and second distinct modulations of the common gene in a sequential manner. Alternatively, the first gate unit and the second gate unit may be activated subsequently simultaneously, and such gates may be preconfigured to still effect the first and second distinct modulations of the common gene in such sequential manner.

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively, or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least 1, 2, 3, 4, 5, or more different gate moieties) and/or a gene regulating moiety (e.g., at least 1, 2, 3, 4, 5, 6, 7, 8, 9, 10, or more different gene regulating moieties). A gate moiety as disclosed herein can comprise a guide nucleic acid molecule (gNA) (e.g., at least 1, 2, 3, 4, 5, or more gNAs). A gene regulating moiety as disclosed herein can comprise a gNA (e.g., at least 1, 2, 3, 4, 5, or more gNAs). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety (e.g., electromagnetic energy) may activate the initial gate unit without directly binding the at least the portion of the initial gate unit. In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the activatable gNA molecule comprises a non-canonical transcription termination sequence (e.g., a polyX sequence, such as a polyU sequence or a polyT sequence), such that a functional gNA molecule is not expressed until a gene encoding the activatable gNA having the non-canonical transcription termination sequence can be modified (e.g., to remove some or all of the transcription termination sequence). Thus, in absence of the modification of the transcription termination sequence, a non-functional variant (e.g., a non-functional fragment) of the gNA may be expressed. In some embodiments, the gNA can be synthetic. In some embodiments, the gNA can have a fluorescent label attached.

In some cases, the non-canonical termination sequence can comprise or consist substantially of a polynucleotide sequence exhibiting at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the polynucleotide sequence of one or more members selected from the group consisting of SEQ ID NOs: 136 and 145, or a complementary sequence thereof.

In some cases, the polynucleotide sequence comprising the non-canonical termination sequence (or a complementary sequence thereof) can have the following structure (II):

M-T′-M′,

wherein: (i) T′ is the non-canonical termination sequence (e.g., polyT) as provided herein; and (ii) M and M′ are as described above for the structure (II).

In some cases, in the pair comprising M and M′ as shown in the structure (II) and/or the structure (III), the pair may form an insulator sequence, as provided herein. Alternatively, the pair may for a stem sequence, as provided herein.

In some cases, in the pair comprising M and M′ as shown in the structure (II), a polynucleotide sequence of M and an additional polynucleotide sequence of M′ can, respectively, exhibit at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the respective pair selected from the group consisting of (1) SEQ ID NO: 117 and SEQ ID NO: 154; (2) SEQ ID NO: 118 and SEQ ID NO: 155; (3) SEQ ID NO: 119 and SEQ ID NO: 156; (4) SEQ ID NO: 120 and SEQ ID NO: 157; (5) SEQ ID NO: 121 and SEQ ID NO: 158; (6) SEQ ID NO: 122 and SEQ ID NO: 159; (7) SEQ ID NO: 123 and SEQ ID NO: 160; (8) SEQ ID NO: 124 and SEQ ID NO: 161; (9) SEQ ID NO: 126 and SEQ ID NO: 162; (10) SEQ ID NO: 127 and SEQ ID NO: 163; (11) SEQ ID NO: 128 and SEQ ID NO: 164; (12) SEQ ID NO: 129 and SEQ ID NO: 165; (13) SEQ ID NO: 130 and SEQ ID NO: 166; (14) SEQ ID NO: 131 and SEQ ID NO: 167; (15) SEQ ID NO: 132 and SEQ ID NO: 168; (16) SEQ ID NO: 133 and SEQ ID NO: 169; (17) SEQ ID NO: 134 and SEQ ID NO: 170; and (18) SEQ ID NO: 135 and SEQ ID NO: 171, or complementary sequence pair thereof.

In some cases, a size of the polyT sequence is greater than or equal to a threshold length, wherein the threshold length is sufficient to reduce expression of the guide nucleic acid molecule from the polynucleotide sequence. Accordingly, a plasmid (e.g., a gate moiety or a gene regulating moiety) can encode an inactivated gNA comprising the polyT sequence that is greater than or equal to the threshold length, and editing of such plasmid to reduce the length of the polyT to below the threshold length can permit expression of the gNA in its entirety without early termination, thereby activating the gNA. In some cases, the polyT sequence comprises at least 5 T. In some cases, the polyT sequence comprises at least 7 T. In some cases, the polyT sequence comprises at least 8 T. In some cases, the polyT sequence comprises at least 10 T. In some cases, the polyT sequence comprises between 5 T and 15 T. In some cases, the polyT sequence comprises one or more additional nucleotides that are not T.

In some cases, a gene regulating moiety (e.g., a guide nucleic acid and/or an endonuclease) can be configured to bind to a target polynucleotide sequence operatively coupled to a target gene in a cell. The target gene can comprise an encoding polynucleotide sequence that encodes a target nucleic acid molecule or a target protein. The target polynucleotide sequence can be a part of the encoding polynucleotide sequence. Alternatively, the target polynucleotide sequence may not be a part of the encoding polynucleotide sequence. For example, the target polynucleotide sequence can be upstream of the encoding polynucleotide sequence (e.g., part of a promoter of the encoding polynucleotide sequence, such as a transcription start site (TSS).

In some embodiments, a proGuide can comprise a target polynucleotide domain at or adjacent to an inactivation polynucleotide sequence (e.g., at or adjacent to 5′ and/or 3′ ends of the inactivation polynucleotide sequences), which target polynucleotide domain can be targeted (e.g., via sequential activation mechanism of the heterologous genetic circuit as provided herein) to modify (e.g., edit, cleave) the inactivation polynucleotide sequence, thereby rendering the proGuide to express an activated guide nucleic acid molecule. The target polynucleotide domain of a proGuide may not exhibit sequence identity to any comparable endogenous polynucleotide sequence in a cell, thereby to avoid inadvertent targeting and modulation of an endogenous target gene.

In some embodiments, the inactivation polynucleotide sequence of the proGuide can be disposed between two target polynucleotide domains, which may or may not be targetable by a common guide nucleic acid sequence. In some cases, the two target polynucleotide domains can be reverse and complementary to one another, such that the inactivation polynucleotide sequence can be modified or cleaved by the same mechanism (e.g., same spacer sequence of a guide nucleic acid molecule).

In some embodiments, a proGuide can comprise a polynucleotide sequence that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members (shown in Table 5) from SEQ ID NOs: 190-191 (e.g., BCL11A targeting), SEQ ID NOs: 192-194 (e.g., BCL11B targeting), SEQ ID NOs: 195-198 (e.g., BCL2 targeting), SEQ ID NOs: 199-206 (e.g., BCL2L1 targeting), SEQ ID NOs: 207-210 (e.g., BMI1 targeting), SEQ ID NOs: 211-214 (e.g., CD34 targeting), SEQ ID NOs: 215-218 (e.g., DLL4 targeting), SEQ ID NOs: 219-224 (e.g., DNMT3B targeting), SEQ ID NOs: 225-227 (e.g., DTX1 targeting), SEQ ID NOs: 228-230 (e.g., EBF1 targeting), SEQ ID NOs: 231-233 (e.g., EOMES targeting), SEQ ID NOs: 234-241 (e.g., ERG targeting), SEQ ID NOs: 242-245 (e.g., ETS1 targeting), SEQ ID NOs: 246-248 (e.g., ETV2 targeting), SEQ ID NOs: 249-252 (e.g., EZH1 targeting), SEQ ID NOs: 253-256 (e.g., FLT1 targeting), SEQ ID NOs: 257-260 (e.g., FOS targeting), SEQ ID NOs: 261-264 (e.g., FOXO1 targeting), SEQ ID NOs: 265-267 (e.g., FOXP3 targeting), SEQ ID NOs: 268-271 (e.g., GATA1 targeting), SEQ ID NOs: 272-278 (e.g., GATA2 targeting), SEQ ID NOs: 279-282 (e.g., GATA3 targeting), SEQ ID NOs:283-289 (e.g., GFI1 targeting), SEQ ID NOs: 290-293 (e.g., GFI1B targeting), SEQ ID NOs: 294-297 (e.g., HES1 targeting), SEQ ID NOs: 298-301 (e.g., HHEX targeting), SEQ ID NOs: 302-304 (e.g., HOXA10 targeting), SEQ ID NOs: 305-307 (e.g., HOXA5 targeting), SEQ ID NOs: 308-311 (e.g., HOXA7 targeting), SEQ ID NOs: 312 (e.g., HOXA9 targeting), SEQ ID NOs: 313-315 (e.g., HOXB4 targeting), SEQ ID NOs: 316-322 (e.g., ID2 targeting), SEQ ID NOs: 323-325 (e.g., ID3 targeting), SEQ ID NOs: 326-329 (e.g., IKZF1 targeting), SEQ ID Nos: 330-336 (e.g., IKZF2 targeting), SEQ ID NOs: 337-340 (e.g., IL7R targeting), SEQ ID NOs: 341-347 (e.g., IRF4 targeting), SEQ ID NOs: 348-351 (e.g., IRF8 targeting), SEQ ID NOs: 352-355 (e.g., KDR targeting), SEQ ID NOs: 356-363 (e.g., LCOR targeting), SEQ ID NOs: 364-371 (e.g., LEF1 targeting), SEQ ID NOs: 372-374 (e.g., LMO2 targeting), SEQ ID NOs: 375-378 (e.g., LYL1 targeting), SEQ ID NOs: 379-382 (e.g., MEF2C targeting), SEQ ID NOs: 383-386 (e.g., MIXL1 targeting), SEQ ID NOs: 387-389 (e.g., MYB targeting), SEQ ID NOs: 390-393 (e.g., MYC targeting), SEQ ID NOs: 394-397 (e.g., NFIL3 targeting), SEQ ID Nos: 398-401 (e.g., NR4A1 targeting), SEQ ID NOs: 402-405 (e.g., PAX5 targeting), SEQ ID NOs: 406-409 (e.g., PGF targeting), SEQ ID NOs: 410-416 (e.g., RORC targeting), SEQ ID NOs: 417-419 (e.g., RUNX1 targeting), SEQ ID NOs: 420-426 (e.g., RUNX3 targeting), SEQ ID NOs: 427-430 (e.g., SATB1 targeting), SEQ ID NOs: 431-434 (e.g., SNAI1 targeting), SEQ ID NOs: 435-438 (e.g., sox17 targeting), SEQ ID NOs: 439-446 (e.g., SOX4 targeting), SEQ ID NOs: 447-448 (e.g., SPI1 targeting), SEQ ID NOs: 449-452 (e.g., SUV39H1 targeting), SEQ ID Nos: 453-460 (e.g., TAL1 targeting), SEQ ID NOs: 461-463 (e.g., TBX21 targeting), SEQ ID NOs: 464-470 (e.g., TCF12 targeting), SEQ ID NOs: 471-478 (e.g., TCF3 targeting), SEQ ID NOs: 479-480 (e.g., TCF7 targeting), SEQ ID NOs: 481-484 (e.g., TOX targeting), SEQ ID Nos: 485-488 (e.g., TOX2 targeting), SEQ ID NOs: 489-492 (e.g., VEGFA targeting), SEQ ID NOs: 493-496 (e.g., VEGFB targeting), SEQ ID NOs: 497-500 (e.g., VEGFC targeting), SEQ ID NOs: 501-504 (e.g., VEGFD targeting), SEQ ID NOs: 505-507 (e.g., ZBTB16 targeting), SEQ ID NOs: 508-511 (e.g., ZBTB17 targeting), SEQ ID NOs: 512-514 (e.g., ZBTB7B targeting), SEQ ID NOs: 515-518 (e.g., ZSCAN4 targeting), or a complementary sequence thereof.

The plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.10%. Alternatively, or in addition to, the distinct modulation of the target gene can be substantially the same (e.g., the same).

The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

One or more target genes as disclosed herein can comprise a cell differentiation regulatory factor, a molecular function regulatory factor, a binding factor, a fusogenic factor, a protein folding chaperone, a protein tag, a RNA folding chaperone, a cell signaling factor, an immune response factor, a sensory receptor, a cell structural factor, a protein binding factor, a cargo receptor, a catalytic factor, or a small molecule sensor.

One or more target genes as disclosed herein can comprise a cell differentiation regulatory factor that comprises a growth factor, a transcription factor, a myogenic regulatory factor, an immune cell regulatory factor, a neuronal regulatory factor, a stem cell differentiation factor, a chondrogenic regulatory factor, an osteogenic regulatory factor, a senescence factor, a sternness factor (e.g., de-differentiation factor), etc.

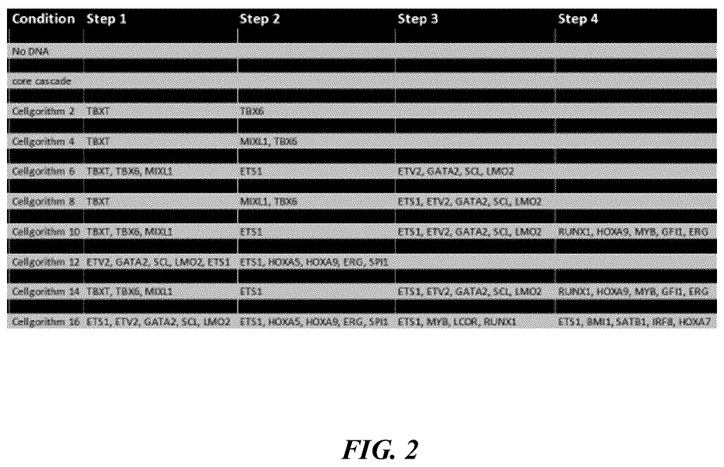

In some cases, the one or more target genes (e.g., one or more immune cell regulatory factors) can comprise MYCN, PTCRA, BCL11B, HHEX, NOTCH1, TCF3, RAG2, DTX1, RUNX1, HOXA9, HOXA5, HOXB4, RAG1, SPI1, EVT2, SCL, ID2, BCL11A, ID3, TCF7, IKZF1, TCF12, RUNX3, LMO2, LEF1, GFI1, LYL1, AQP3, MEIS1, GATA2, GATA3, HES1, ST18, NR4A1, C20ORF100, IKAROS, SPIB, RORC, TCF1, LCOR, IRF8, SATB1, BMI1, MYC, AHR, MIXL1, FOXO1, NOTCH1, NOTCH3, IL-2, IL4, IL7, IL15, EBF1, PAX5, TAL1, MYB, ERG, HHEX, E4PB4, GFI1B, PTA, TBXT, TBX6, TBX21, ETS1, ETS2, or MEF2C. For example, one or more target genes for inducing hematopoietic stem cell differentiation can be selected from the group consisting of TBXT, TBX6, MIXL1, ETS1, ETV2, GATA2, SCL, LMO2, HOXA5, HOXA9, ERG, SPI1, MYB, RUNX1, HOXB4, GFI1, LCOR, IRF8, SATB1, and BMI1 (FIG. 2). In some cases, the one or more target genes (e.g., one or more hematopoietic stem cell differentiation factors) may not comprise TBXT, TBX6, and/or MIXL1.

In some cases, the one or more target genes (e.g., one or more stem cell differentiating factors) can comprise HOXB4, NOTCH1, SOX11, SOX17, RUNX1, GATA2, FLI1, ERG, WNT, HNF1, HNF3, HNF4, CDX2, and LIN28A.

In some cases, the one or more target genes (e.g., one or more stemness factors) can comprise HES1, HES5, CBF1, SOC2, HMGA2, OLIG2, ID2, ID4, HESR1, HESR2, GLI1, GLI2, GLI3, SOXB, and BMI1.

In some cases, the one or more target genes can comprise T-box transcription factors (TBX genes). TBX transcription factors are involved in development. T-box proteins have relatively large DNA-binding domains. Non-limiting examples of TBX transcription factors can include TBX1, TBX2, TBX3, TBX4, TBX5, TBX6, TBX10, TBX15, TBX18, TBX19, TBX20, TBX21, TBX22, and TBXT (Brachyury protein). In some cases, the one or more target genes may not comprise one or more TBX transcription factors (e.g., TBXT or TBX6).

In some cases, the plurality of gate units is preconfigured such that one of the plurality of gate units is activatable, upon the activation of the heterologous genetic circuit, to modulate the expression and/or activity level of a TBX gene. In some cases, the first gate unit is activatable to modulate the expression and/or activity level of a TBX gene. Alternatively, or in addition to, a second gate unit is activatable to modulate the expression and/or activity level of a TBX gene. Alternatively, or in addition to, a gate unit is activated before the first gate unit to modulate the expression and/or activity level of a TBX gene. Alternatively, or in addition to, a gate unit is activated between the first gate unit and the second gate unit to modulate the expression and/or activity level of a TBX gene.

In some cases, the plurality of gate units is preconfigured such that one of the plurality of gate units is activatable, upon the activation of the heterologous genetic circuit, to modulate the expression and/or activity level of TBXT. In some cases, the first gate unit is activatable to modulate the expression and/or activity level of TBXT. Alternatively, or in addition to, a second gate unit is activatable to modulate the expression and/or activity level of TBXT. Alternatively, or in addition to, a gate unit is activated before the first gate unit to modulate the expression and/or activity level of TBXT. Alternatively, or in addition to, a gate unit is activated between the first gate unit and the second gate unit to modulate the expression and/or activity level of TBXT.

In some cases, the plurality of gate units is preconfigured such that one of the plurality of gate units is activatable, upon the activation of the heterologous genetic circuit, to modulate the expression and/or activity level of TBX6. In some cases, the first gate unit is activatable to modulate the expression and/or activity level of TBX6. Alternatively, or in addition to, a second gate unit is activatable to modulate the expression and/or activity level of TBX6. Alternatively, or in addition to, a gate unit is activated before the first gate unit to modulate the expression and/or activity level of TBX6. Alternatively, or in addition to, a gate unit is activated between the first gate unit and the second gate unit to modulate the expression and/or activity level of TBX6.

In some cases, the one or more target genes can comprise a basic helix-loop-helix transcription factor (bHLH gene). bHLH transcription factors are involved in the regulation of the cell cycle and many other developmental processes. bHLH proteins have a basic helix-loop-helix protein structure. Non-limiting examples of bHLH transcription factors can include AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPAS1, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOA1, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTF1A, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2.

In some cases, the plurality of gate units is preconfigured such that one of the plurality of gate units is activatable, upon the activation of the heterologous genetic circuit, to modulate the expression and/or activity level of a bHLH gene. In some cases, the first gate unit is activatable to modulate the expression and/or activity level of a bHLH gene. Alternatively, or in addition to, a second gate unit is activatable to modulate the expression and/or activity level of a bHLH gene. Alternatively, or in addition to, a gate unit is activated before the first gate unit to modulate the expression and/or activity level of a bHLH gene. Alternatively, or in addition to, a gate unit is activated between the first gate unit and the second gate unit to modulate the expression and/or activity level of a bHLH gene.

In some cases, the plurality of gate units is preconfigured such that one of the plurality of gate units is activatable, upon the activation of the heterologous genetic circuit, to modulate the expression and/or activity level of SCL. In some cases, the first gate unit is activatable to modulate the expression and/or activity level of SCL. Alternatively, or in addition to, a second gate unit is activatable to modulate the expression and/or activity level of SCL. Alternatively, or in addition to, a gate unit is activated before the first gate unit to modulate the expression and/or activity level of SCL. Alternatively, or in addition to, a gate unit is activated between the first gate unit and the second gate unit to modulate the expression and/or activity level of SCL.

In some cases, the one or more target genes can comprise a SRY-related box transcription factor (SOX gene). SOX transcription factors are involved in developmental regulation. Non-limiting examples of SOX transcription factors can include SOX1, SOX2, SOX3, SOX4, SOX5, SOX6, SOX7, SOX8, SOX9, SOX10, SOX11, SOX12, SOX13, SOX14, SOX15, SOX17, SOX18, SOX21, SOX30, and SRY.

In some cases, the one or more target genes can comprise SOX group A, comprising SRY. In some cases, the one or more target genes can comprise SOX group B1, comprising SOX1, SOX2 and/or SOX3. In some cases, the one or more target genes can comprise SOX group B2, comprising SOX14 and/or SOX21. In some cases, the one or more target genes can comprise SOX group C, comprising SOX4, SOX11 and/or SOX12. In some cases, the one or more target genes can comprise SOX group D, comprising SOX5, SOX6, and/or SOX13. In some cases, the one or more target genes can comprise SOX group E, comprising SOX8, SOX9, and/or SOX10. In some cases, the one or more target genes can comprise SOX group F, comprising SOX7, SOX 17, and/or SOX18. In some cases, the one or more target genes can comprise SOX group G, comprising SOX15. In some cases, the one or more target genes can comprise SOX group H, comprising SOX30.

In some cases, the one or more target genes can comprise a forkhead box (FOX). FOX are transcription factors that play a role in regulating the expression of genes involved in cell growth, proliferation, differentiation, and longevity. Some FOX genes can bind chromatin during cell differentiation processes. Non-limiting examples of FOX genes can include FOXA, FOXB, FOXC, FOXD, FOXE, FOXF, FOXG, FOXH, FOXI, FOXJ, FOXK, FOXL, FOXM, FOXN, FOXO, FOXP, FOXQ, FOXR, and FOXS.

In some cases, the one or more target genes can comprise an erythroblast transformation specific (ETS) gene. ETS are transcription factors unique to animals and are implicated in tissue development. Non-limiting examples of ETS genes can include ELF1, ELF2 (NERF) ELF4 (MEF) GABPU, ERG, FLI1, FEV, ERF (PE2), ETV3 (PE1), ELF3 (ESE1/ESX), ELF5 (ESE2), ESE3 (EHF), ETS1, ETS2, SPDEF (PDEF/PSE), ETV4 (PEA3/E1AF), ETV5 (ERM), ETV1 (ER81), ETV2 (ER71), SPI1 (PU.1), SPIB, SPIC, ELK1, ELK4 (SAP1), ELK3 (NET/SAP2), ETV6 (TEL), and ETV7 (TEL2).

In some cases, the one or more target genes can comprise a collagen. Collagens are fibrous proteins and are the major elements of skin, bone, tendon, cartilage, blood vessels, and teeth. Collagens form insoluble fibers of high tensile strength. Non-limiting examples of collagen genes can include COL1A1, COL1A2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4P1, COL6A4P2, COL6A5, COL6A6, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, COL26A1, COL27A1, and COL28A1.

In some cases, the one or more target genes can comprise a homeobox gene. Homeobox genes are genes that regulate, for example, large-scale anatomical features in the early stages of embryonic development. Types of homeobox genes include HOX genes, LIM genes, PAX genes, POU genes, CERS genes, HNF genes, SINE genes, CUT genes, ZF genes, paraHOX genes, DLX genes, TALE genes, PRD genes, and NKL genes. Non-limiting examples of homeobox genes can include HOXA1, HOXA2, HOXA3, HOXA4, HOXA5, HOXA6, HOXA7, HOXA9, HOXA10, HOXA11, HOXA13, HOXB1, HOXB2, HOXB3, HOXB4, HOXB5, HOXB6, HOXB7, HOXB8, HOXB9, HOXB13, HOXC4, HOXC5, HOXC6, HOXC8, HOXC9, HOXC10, HOXC11, HOXC12, HOXC13, HOXD1, HOXD3, HOXD4, HOXD8, HOXD9, HOXD10, HOXD11, HOXD12, HOXD13, CDX1, CDX2, CDX4, GSX1, GSX2, PDX1, EVX1, EVX2, GBX1, GBX2, MEOX1, MEOX2, MNX1, DLX1, DLX2, DLX3, DLX4, DLX5, DLX6, IRX1, IRX2, IRX3, IRX4, IRX5, IRX6, MEIS1, MEIS2, MEIS3, MKX, PBX1, PBX2, PBX3, PBX4, PKNOX1, PKNOX2, TGIF1, TGIF2, TGIF2LX, TGIF2LY, ISL1, ISL2, LHX1, LHX2, LHX3, LHX4, LHX5, LHX6, LHX8, LHX9, LMX1A, LMX1B, HDX, POU1F1, POU2F1, POU2F2, POU2F3, POU3F1, POU3F2, POU3F3, POU3F4, POU4F1, POU4F2, POU4F3, POU5F1, POU5F1P1, POU5F1P4, POU5F2, POU6F1, POU6F2, LASS2, LASS3, LASS4, LASS5, LASS6, HMBOX1, HNF1A, HNF1B, SIX1, SIX2, SIX3, SIX4, SIX5, SIX6, ONECUT1, ONECUT2, ONECUT3, CUX1, CUX2, SATB1, SATB2, ADNP, ADNP2, TSHZ1, TSHZ2, TSHZ3, ZEB1, ZEB2, ZFHX2, ZFHX3, ZFHX4, ZHX1, HOMEZ, ALX1 (CART1), ALX3, ALX4, ARGFX, ARX, DMBX1, DPRX, DRGX, DUXA, DUXB, DUX(1, 2, 3, 4, 4c, 5), ESX1, GSC, GSC2, HESX1, HOPX, ISX, LEUTX, MIXL1, NOBOX, OTP, OTX1, OTX2, CRX, PAX2, PAX3, PAX4, PAX5, PAX6, PAX7, PAX8, PHOX2A, PHOX2B, PITXT, PITX2, PITX3, PROP1, PRRX1, PRRX2, RAX, RAX2, RHOXF1, RHOXF2/2B, SEBOX, SHOX, SHOX2, TPRX1, UNCX, VSXT, VSX2, BARHL1, BARHL2, BARX1, BARX2, BSX, DBX1, DBX2, EMX1, EMX2, EN1, EN2, HHEX, HLX1, LBX1, LBX2, MSX1, MSX2, NANOG, NOTO, TLX1, TLX2, TLX3, TSHZ1, TSHZ2, TSHZ3, VAX1, VAX2, VENTX, NKX2-1, NKX2-4, NKX2-2, NKX2-8, NKX3-1, NKX3-2, NKX2-3, NKX2-5, NKX2-6, HMX1, HMX2, HMX3, NKX6-1, NKX6-2, and NKX6-3. In some cases, the one or more target genes may not comprise a homeobox gene (e.g., MIXL1).

In some cases, the plurality of gate units is preconfigured such that one of the plurality of gate units is activatable, upon the activation of the heterologous genetic circuit, to modulate the expression and/or activity level of a homeobox gene. In some cases, the first gate unit is activatable to modulate the expression and/or activity level of a homeobox gene. Alternatively, or in addition to, a second gate unit is activatable to modulate the expression and/or activity level of a homeobox gene. Alternatively, or in addition to, a gate unit is activated before the first gate unit to modulate the expression and/or activity level of a homeobox gene. Alternatively, or in addition to, a gate unit is activated between the first gate unit and the second gate unit to modulate the expression and/or activity level of a homeobox gene.

In some cases, the plurality of gate units is preconfigured such that one of the plurality of gate units is activatable, upon the activation of the heterologous genetic circuit, to modulate the expression and/or activity level of MIXL1. In some cases, the first gate unit is activatable to modulate the expression and/or activity level of MIXL1. Alternatively or in addition to, a second gate unit is activatable to modulate the expression and/or activity level of MIXL1. Alternatively, or in addition to, a gate unit is activated before the first gate unit to modulate the expression and/or activity level of MIXL1. Alternatively, or in addition to, a gate unit is activated between the first gate unit and the second gate unit to modulate the expression and/or activity level of MIXL1.

In some cases, the one or more target genes can comprise a GATA gene. GATA genes are transcription factors characterized by their ability to bind to the DNA sequence “GATA.” Non-limiting examples of GATA genes can include GATA1, GATA2, GATA3, GATA4, GATA5, and GATA6.

In some cases, the plurality of gate units is preconfigured such that one of the plurality of gate units is activatable, upon the activation of the heterologous genetic circuit, to modulate the expression and/or activity level of a GATA gene. In some cases, the first gate unit is activatable to modulate the expression and/or activity level of a GATA gene. Alternatively, or in addition to, a second gate unit is activatable to modulate the expression and/or activity level of a GATA gene. Alternatively, or in addition to, a gate unit is activated before the first gate unit to modulate the expression and/or activity level of a GATA gene. Alternatively, or in addition to, a gate unit is activated between the first gate unit and the second gate unit to modulate the expression and/or activity level of a GATA gene.

In some cases, the plurality of gate units is preconfigured such that one of the plurality of gate units is activatable, upon the activation of the heterologous genetic circuit, to modulate the expression and/or activity level of GATA2. In some cases, the first gate unit is activatable to modulate the expression and/or activity level of GATA2. Alternatively, or in addition to, a second gate unit is activatable to modulate the expression and/or activity level of GATA2. Alternatively, or in addition to, a gate unit is activated before the first gate unit to modulate the expression and/or activity level of GATA2. Alternatively, or in addition to, a gate unit is activated between the first gate unit and the second gate unit to modulate the expression and/or activity level of GATA2.

In some cases, a guide nucleic acid (gNA) molecule as provided herein can be configured to bind a target gene (e.g., a coding region or a non-coding region of the target gene), to modulate expression and/or activity of the target gene. The gNA molecule can comprise a polynucleotide sequence (e.g., a spacer sequence) exhibiting specific binding to a target polynucleotide sequence of the target gene. The polynucleotide sequence of the gNA molecule can exhibit at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to the polynucleotide sequence of any one of the SEQ ID NOs. 1-112 (e.g., that of any one of the SEQ ID NOs. 13-16 for targeting ETS1). The polynucleotide sequence of the gNA molecule that binds to the target gene can comprise at least or up to about 10 nucleotides, at least or up to about 11 nucleotides, at least or up to about 12 nucleotides, at least or up to about 13 nucleotides, at least or up to about 14 nucleotides, at least or up to about 15 nucleotides, at least or up to about 16 nucleotides, at least or up to about 17 nucleotides, at least or up to about 18 nucleotides, at least or up to about 19 nucleotides, at least or up to about 20 nucleotides, at least or up to about 21 nucleotides, at least or up to about 22 nucleotides, at least or up to about 23 nucleotides, at least or up to about 24 nucleotides, or at least or up to about 35 nucleotides.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, mesodermal stem cells) into hematopoietic lineage cells (e.g., hematopoietic stem cells or hematopoietic precursor cells) whereby at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% of the resulting cells generated by using the heterologous genetic circuit as disclosed herein are hematopoietic lineage cells.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, mesodermal stem cells) into hematopoietic lineage cells (e.g., hematopoietic stem cells or hematopoietic precursor cells) whereby at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% of the resulting cells generated by using the heterologous genetic circuit as disclosed herein are the target cell type. In some embodiments, the target cell can be ascertained by characterizing one or more cell surface markers which can include KDR, CD34, CD43, CD45, and CD309.

In some cases, use of the heterologous genetic circuit as disclosed herein can be used to differentiate stem cells (e.g., pluripotent stem cells (PSC), erythro-myeloid progenitor (EMP) cells, mesogenic progenitor cells) into hematopoietic lineage cells (e.g., hematopoietic stem cells or hematopoietic precursor cells), e.g., in absence of one, two, or all of feeder cells, serum, and one or more exogenous factors (e.g., one or more exogenous growth factors. In some cases, the one or more exogenous factors may otherwise be required to effect differentiation of the stem cells into the hematopoietic lineage cells in absence of the heterologous genetic circuit as provided herein. Non-limiting examples of the one or more exogenous factors can include a bone morphogenetic protein (BMP), a vascular endothelial growth factor (VEGF), a fibroblast growth factor (FGF), a stem cell factor (SCF), thrombopoietin (TPO), FMS-like tyrosine kinase receptor ligand (FLTL), and/or interleukin (IL).

Non-limiting examples of BMP can include BMP1, BMP1b, BMP2, BMP2A, BMP2B, BMP3, BMP4, BMP5, BMP6, BMP7, BMP8A, and BMP8B. In some embodiments, the BMP comprises BMP4. Non-limiting examples of VEGF can include VEGF-A, VEGF-B, VEGF-C, VEGF-D, and PGF. Non-limiting examples of FGF can include FGF1, FGF2 (basic FGF or bFGF), FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23. Non-limiting examples of FLTL can include FLTL-1, FLTL-2, FLTL-3, and FLTL-4. Non-limiting examples of the IL can include IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36.

Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹¹, at least about 2×10¹⁵, at least about 5×10¹⁵, or more hematopoietic lineage cells (e.g., hematopoietic stem cells or hematopoietic precursor cells) from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or fewer pluripotent stem cells (e.g., human PSCs).

Such generation of hematopoietic lineage cells (e.g., hematopoietic stem cells or hematopoietic precursor cells) by using the heterologous genetic circuit as disclosed herein can be achieved within the span of at most about 60 days, at most about 55 days, at most about 50 days, at most about 45 days, at most about 40 days, at most about 35 days, at most about 30 days, at most about 25 days, at most about 20 days, at most about 15 days, at most about 10 days, at most about 9 days, at most about 8 days, at most about 7 days, at most about 6 days, at most about 5 days, at most about four days, at most about three days, at most about 2 days, or less. When injected into mice, the resulting hematopoietic lineage cells (e.g., hematopoietic stem cells or hematopoietic precursor cells) generated by using the heterologous genetic circuit as disclosed herein can extend mouse lifespan at least about 30%, at least about 40%, at least about 50%, at least about 60%, at least about 70%, at least about 80%, at least about 90%, at least about 100%, or more.

In some cases, a plurality of a first type of cell can be converted to a plurality of a second type of cell within at most about 20 days, within at most about 19 days, within at most about 18 days, within at most about 17 days, within at most about 16 days, within at most about 15 days, within at most about 14 days, within at most about 13 days, within at most about 12 days, within at most about 11 days, within at most about 10 days, within at most about 9 days, within at most about 8 days, within at most about 7 days, within at most about 6 days, within at most about 5 days, within at most about 4 days, within at most about 3 days, within at most about 2 days, or within at most about 1 day following the culturing (e.g., ex vivo culturing) of the stem cells. In some cases, the conversion rate from the plurality of the first type cells to the plurality of the second type of cells can be at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100%.

In some cases, a plurality of stem cells can be converted to a plurality of hematopoietic lineage cells within at most about 20 days, within at most about 19 days, within at most about 18 days, within at most about 17 days, within at most about 16 days, within at most about 15 days, within at most about 14 days, within at most about 13 days, within at most about 12 days, within at most about 11 days, within at most about 10 days, within at most about 9 days, within at most about 8 days, within at most about 7 days, within at most about 6 days, within at most about 5 days, within at most about 4 days, within at most about 3 days, within at most about 2 days, or within about 1 day following the culturing (e.g., ex vivo culturing) of the stem cells. In some cases, the conversion rate from the plurality of stem cells to the plurality of hematopoietic lineage cells can be at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100%.

In some cases, a plurality of stem cells can be converted to a plurality of hematopoietic lineage cells, wherein the hematopoietic lineage cells are CD45+ (e.g., CD34+CD43−CD45+), within at most about 20 days, within at most about 19 days, within at most about 18 days, within at most about 17 days, within at most about 16 days, within at most about 15 days, within at most about 14 days, within at most about 13 days, within at most about 12 days, within at most about 11 days, within at most about 10 days, within at most about 9 days, within at most about 8 days, within at most about 7 days, within at most about 6 days, within at most about 5 days, within at most about 4 days, within at most about 3 days, within at most about 2 days, or within about 1 day following the culturing (e.g., ex vivo culturing) of the stem cells. In some cases, the conversion rate from the plurality of stem cells to the plurality of hematopoietic lineage cells wherein the hematopoietic lineage cells are CD45+(e.g., CD34+CD43−CD45+) can be at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100%.

In some cases, a plurality of stem cells can be converted to a plurality of hematopoietic lineage cells wherein the hematopoietic lineage cells are CD34+ within at most about 20 days, within at most about 19 days, within at most about 18 days, within at most about 17 days, within at most about 16 days, within at most about 15 days, within at most about 14 days, within at most about 13 days, within at most about 12 days, within at most about 11 days, within at most about 10 days, within at most about 9 days, within at most about 8 days, within at most about 7 days, within at most about 6 days, within at most about 5 days, within at most about 4 days, within at most about 3 days, within at most about 2 days, or within about 1 day following the culturing (e.g., ex vivo culturing) of the stem cells. In some cases, the conversion rate from the plurality of stem cells to the plurality of hematopoietic lineage cells wherein the hematopoietic lineage cells are CD34+ can be at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or about 100%.

In some cases, use of the heterologous genetic circuit in the stem cells (e.g., pluripotent stem cells (PSC), erythro-myeloid progenitor (EMP) cells, mesogenic progenitor cells) can induce the stem cells to differentiate into hematopoietic progenitor cells in absence of feeder cells. In some cases, use of the heterologous genetic circuit in the stem cells (e.g., pluripotent stem cells (PSC), erythro-myeloid progenitor (EMP) cells, mesogenic progenitor cells) can induce the stem cells to differentiate into hematopoietic progenitor cells in absence of serum. In some cases, use of the heterologous genetic circuit in the stem cells (e.g., pluripotent stem cells (PSC), erythro-myeloid progenitor (EMP) cells, mesogenic progenitor cells) can induce the stem cells to differentiate into hematopoietic progenitor cells in absence of one or more exogenous factors (e.g., one or more exogenous growth factors. In some cases, use of the heterologous genetic circuit as disclosed herein can be used to differentiate stem cells (e.g., pluripotent stem cells (PSC), erythro-myeloid progenitor (EMP) cells, mesogenic progenitor cells) into hematopoietic progenitor cells e.g., in the absence of one or both embryoid bodies and serum. The resulting hematopoietic progenitor cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population.

In some cases, a target gene may be subjected to at least two distinct modulations comprising a first modulation and a second modulation. Timing of the first modulation and the second modulation can be controlled (e.g., as predetermined by the design of the heterologous genetic circuit). For example, the onset of the second modulation (e.g., by at least a portion of the second gate unit, such as the second gene regulation moiety) can occur subsequent to the onset of the first modulation (e.g., by at least a portion of the first gate unit, such as the first gene regulating moiety) by at least about 1 second, at least about 2 seconds, at least about 3 seconds, at least about 4 seconds, at least about 5 seconds, at least about 6 seconds, at least about 7 seconds, at least about 8 seconds, at least about 9 seconds, at least about 10 seconds, at least about 20 seconds, at least about 30 seconds, at least about 40 seconds, at least about 50 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 20 hours, at least about 1 day, at least about 2 days, at least about 3 days, at least about 4 days, at least about 5 days, at least about 6 days, at least about 7 days, at least about 8 days, at least about 9 days, or at least about 10 days. The onset of the second modulation (e.g., by at least a portion of the second gate unit, such as the second gene regulation moiety) can occur subsequent to the onset of the first modulation (e.g., by at least a portion of the first gate unit, such as the first gene regulation moiety) by at most about 10 days, at most about 9 days, at most about 8 days, at most about 7 days, at most about 6 days, at most about 5 days, at most about 4 days, at most about 3 days, at most about 2 days, at most about 1 day, at most about 20 hours, at most about 10 hours, at most about 9 hours, at most about 8 hours, at most about 7 hours, at most about 6 hours, at most about 5 hours, at most about 4 hours, at most about 3 hours, at most about 2 hours, at most about 1 hours, at most about 50 minutes, at most about 40 minutes, at most about 30 minutes, at most about 20 minutes, at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 1 minutes, at most about 50 seconds, at most about 40 seconds, at most about 30 seconds, at most about 20 seconds, at most about 10 seconds, at most about 9 seconds, at most about 8 seconds, at most about 7 seconds, at most about 6 seconds, at most about 5 seconds, at most about 4 seconds, at most about 3 seconds, at most about 2 seconds, or at most about 1 second.

In some cases, a number of gate units that need to be activated (e.g., sequentially activated) between the activation of the first modulation by the first gate unit and the later activation of the second modulation by the second gate unit can at least in part determine (e.g., substantially determine) the timing between the first modulation and the second modulation. Upon activation of the first modulation of the target gene by the first gate unit, at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more additional gate units may need to be activated (e.g., sequentially activated) to activate the second gate unit for inducing the second modulation. Upon activation of the first modulation of the target gene by the first gate unit, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 additional gate units may need to be activated (e.g., sequentially activated) to activate the second gate unit for inducing the second modulation.

The outcome of a cell can comprise the regulation of a plurality of target genes. For example, the outcome can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The outcome can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic circuit and/or (ii) the activating moiety of the heterologous genetic circuit.

The plurality of gate units can operate sequentially (e.g., each of the plurality of gate units is activated in a sequential manner). For example, a gate unit of the plurality to be activated to activate a subsequent gate unit of the plurality. Sequential operation of the gate units can be linear. Alternatively, sequential operation of the gate units can route back on one another as inputs to form a loop. For example, a plurality of the gate units can induce a feedback loop such as a positive feedback loop or a negative feedback loop.

In some embodiments of any one of the systems disclosed herein, the first gate unit can comprise a first gene regulating moiety that can be activatable to exhibit specific binding to the target gene to induce a first distinct modulation. Alternatively, or in addition to, the first gate unit can comprise a first gene regulating moiety that can be activatable to exhibit non-specific binding to the target gene to induce the first distinct modulation.

The first distinct modulation can induce a change (e.g., increase or decrease) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. The first distinct modulation can induce a change (e.g., increase or decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.10%, or less.

The first distinct modulation as disclosed herein (e.g., induced by the first gate unit) can induce a change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The first distinct modulation can induce a change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Subsequently, a second distinct modulation as disclosed herein (e.g., induced by the second gate unit) can induce an additional change (e.g., increase, decrease, or selective attenuation) in the expression and/or activity level of the target gene by at least about 0.10%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce an additional change (e.g., increase or decrease) in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%.

The additional change via the second distinct modulation can induce an additional change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The second distinct modulation can induce an additional change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

The additional change via the second distinct modulation can occur when the expression and/or activity level of the target gene reaches a target level via action of the first distinct modulation, e.g., by design of the heterologous genetic circuit.

The additional change via the second distinct modulation can occur when the expression and/or activity level of the target gene is changed (e.g., increased or decreased) via action of the first distinct modulation by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The additional change via the second distinct modulation can occur when the expression and/or activity level of the target gene is changed (e.g., increased or decreased) via action of the first distinct modulation by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Alternatively, or in addition to, a second distinct modulation as disclosed herein (e.g., induced by the second gate unit) can induce a change (e.g., increase or decrease) in the expression and/or activity level of an additional target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce a change (e.g., increase or decrease) in the expression and/or activity level of the additional target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. A cell can comprise a prokaryotic cell, a eukaryotic cell, or an artificial cell.

In some embodiments, the second plurality comprises hemogenic endothelium cells that are KDR− and CD34+, further optionally wherein within about 14 days or about 7 days following the contacting, a conversion rate from the first plurality to the second plurality is at least or at most about 2%, 3%, 4%, 5%, 6%, 7%, 8%, 9%, 10%, 11%, 12%, 13%, 14%, 15%, 16%, 17%, 18%, 19%, or 20%. In some embodiments, the conversion rate from the first plurality of the second plurality via the heterologous genetic circuit is greater than that in absence of the heterologous genetic circuit by at least or at most about 0.5-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold.

In some embodiments, the second plurality comprises hematopoietic cells (HPCs) that are CD34+ and CD43+, further optionally wherein within about 14 days or about 7 days following the contacting, a conversion rate from the first plurality to the second plurality is at least or at most about 2%, 3%, 4%, 5%, 6%, 7%, 8%, 9%, 10%, 11%, 12%, 13%, 14%, 15%, 16%, 17%, 18%, 19%, or 20%. In some embodiments, the conversion rate from the first plurality of the second plurality via the heterologous genetic circuit is greater than that in absence of the heterologous genetic circuit by at least or at most about 0.5-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold.

In some embodiments, the second plurality comprises hematopoietic cells (HPCs) that are CD34+ and CD45+, further optionally wherein within about 14 days or about 7 days following the contacting, a conversion rate from the first plurality to the second plurality is at least or at most about 2%, 3%, 4%, 5%, 6%, 7%, 8%, 9%, 10%, 11%, 12%, 13%, 14%, 15%, 16%, 17%, 18%, 19%, or 20%. In some embodiments, the conversion rate from the first plurality of the second plurality via the heterologous genetic circuit is greater than that in absence of the heterologous genetic circuit by at least or at most about 0.5-fold, 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold.

A cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, a secretory cell, a blood cell, a germ cell, a nurse cell, a storage cell, an enteroendocrine cell, a pituitary cell, a neurosecretory cell, a duct cell, an odontoblast, a cementoblast, a glial cell, an interstitial cell, a mesodermal stem cell, a endodermal stem cell, an ectodermal stem cell, a hematopoietic progenitor cell, a mesodermal progenitor cell, a mesenchymal stem cell (MSC), or a erythro-myeloid progenitor (EMP) cell.

Non-limiting examples of such a cell can include lymphoid cells, such as B cell, T cell (Cytotoxic T cell, Natural Killer T cell, Regulatory T cell, T helper cell), Natural killer cell, cytokine induced killer (CIK) cells (see e.g., US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

A stem cell can comprise an induced pluripotent stem cell (iPSC), a pluripotent stem cell (PSC), an embryonic stem cell (ESC), a mesenchymal stem cell (MSC), a erythro-myeloid progenitor (EMP) cell, a mesogenic progenitor cell, a hematopoietic stem cell (HSC), a hematopoietic progenitor cell, a muscle stem cell, a neural stem cell, an epithelial stem cell, an epidermal stem cell, a mammary stem cell, an intestinal stem cell, a neural crest stem cell, or a testicular stem cell.

The stem cells as provided herein (e.g., pluripotent stem cells) can be engineered to disrupt expression of a T cell receptor (TCR) (e.g., TCR alpha, TCR beta, CD3 gamma, CD3 delta, CD3 epsilon, and/or CD247) and/or a major histocompatibility complex (MHC) (e.g., MHC class I, MHC class II, and/or MHC class III, wherein human MHC may be referred to as human leukocyte antigen (HLA)). For example, a gene encoding at least a portion of the TCR of the MHC can be edited (e.g., mutated through nonhomologous end-joining (NHEJ) or homologous recombination (HR)) to disrupt the expression. Disrupting the expression of the TCR and/or the MHC can reduce or eliminate (i) graft-versus-host disease (GvHD) and/or (ii) host-versus-graft disease (HvGD) upon administration of the differentiated cells as provided herein to a subject in need thereof. For example, disrupting the expression of the TCR and/or the MHC can reduce or eliminate the effects of administering the differentiated cells as allogeneic cells (e.g., allogeneic T cells) to a subject that is HLA-mismatched (or partially mismatched). Alternatively, the differentiated cells can be administered as allogeneic cells to a subject that is HLA-matched (e.g., without disruption expression of the TCR and/or the MHC).

Disruption of the expression of the TCR and/or the MHC can be performed prior to, simultaneously with, or subsequent to the conversion of the plurality of stem cells (e.g., pluripotent stem cells) to hematopoietic lineage cells (e.g., hematopoietic stem cells). Disruption of the expression of the TCR and/or the MHC can be performed prior to, simultaneously with, or subsequent to the introduction (e.g., expression) of the heterologous genetic circuit to the plurality of stem cells. Disruption of the expression of the TCR and/or the MHC can be performed prior to, simultaneously with, or subsequent to the activation of the heterologous genetic circuit in the plurality of stem cells. In some cases, disruption of the expression of the TCR and/or the MHC can be performed subsequent to generating the hematopoietic lineage cells.

Various aspects of the present disclosure provide engineered cells that are programmed to induce a desired expression and/or activity level (or profile thereof) of one or more target genes in a cell.

In some embodiments, the engineered cell (e.g., the engineered hematopoietic progenitor cell, the engineered immune cell) of the present disclosure can be generated from an isolated stem cell (e.g., isolate ESCs, iPSCs, EMPs, etc.). The heterologous genetic circuit and/or its components (e.g., gate units, gate moieties, activating moieties, etc.), as disclosed herein, can be introduced during any stage (or cellular state) between and including (a) the isolated stem cell and (b) the differentiated hematopoietic progenitor cell state thereof.

In some cases, the engineered cells hematopoietic progenitor cells can be derived from PSCs, and the heterologous genetic circuit and/or its components (e.g., the heterologous gate units, the heterologous activating moieties, the heterologous gate moieties, etc.) can be introduced to the cell at (A) the PSC state, (B) the mesodermal stem cell state, (C) the hematopoietic progenitor cell state or (D) any other intermediary cell states. The heterologous genetic circuit and/or its components (e.g., the heterologous gate units, the heterologous activating moieties, the heterologous gate moieties, etc.) can be introduced to the cell once during one of (A), (B), (C), or (D). Alternatively, the heterologous genetic circuit and/or its components (e.g., the heterologous gate units, the heterologous activating moieties, the heterologous gate moieties, etc.) can be introduced to the cell multiple times during two, three, or all of (A), (B), (C), or (D).

The engineered cell (e.g., the engineered hematopoietic progenitor cell) of the present disclosure can be used (e.g., administered) to treat a subject in need thereof. The subject can have or can be suspected of having a condition, such as a disease (e.g., cancer). A cell (e.g., a stem cell or a differentiated cell) can be obtained from the subject and such cell can be cultured ex vivo and genetically modified to generate any subject engineered cell (e.g., any immune cell) as disclosed herein. Subsequently, the engineered immune cell can be administered to the subject for adaptive immunotherapy. Thus, the engineered cell can be autologous to the subject in need thereof. Alternatively, the engineered cell can be allogeneic to the subject (e.g., allogeneic stem cell transplantation, allogeneic adoptive immunotherapy, etc.).

A cell or construct of the present disclosure can be used (e.g., administered) in a pharmaceutical formulation. A pharmaceutical formulation can further comprise an additional therapeutic agent. An additional therapeutic agent can comprise a chemotherapeutic agent, an immunosuppressing agent, and/or an antibiotic agent.

A chemotherapeutic agent, also known as an antineoplastic agent, is a type of cancer treatment used to directly or indirectly inhibit the growth and proliferation of cancer cells. A chemotherapeutic agent can be an alkylating agent (e.g., an oxazaphosphorine, a nitrogen mustard, an imidazotetrazine, a nitrosourea, an alkyl sulfonate, a hydrazine, or a platinum based agent), an antimetabolite (e.g., an antifolate, a pyrimidine antagonist, a purine antagonist, or a ribonuclease reductase inhibitor), a topoisomerase inhibitor (e.g., a topoisomerase I inhibitor or a topoisomerase II inhibitor), an antibiotic (e.g., a bleomycin, an actinomycin D, an anthracycline, or a mitomycin), a mitotic inhibitor (e.g., a vinca alkaloid, a taxane, or a nontaxane microtubule inhibitor), a protein kinase inhibitor (e.g., a tyrosine kinase inhibitor, a MEK inhibitor, a CDK inhibitor), a proteasome inhibitor, or a PARP inhibitor.

An immunosuppressing agent is an agent that decreases an immune response. Non-limiting examples of immunosuppressing agents can include steroids (e.g., prednisone, methylprednisolone, dexamethasone), colchicine, hydroxychloroquine, sulfasalazine, dapsone, methotrexate, mycophenolate mofetil, azathioprine, anti-IL-1 biologics, anti-TNF biologics, anti-IL-6 biologics, B cell growth factor targeting biologics, T cells, cytokines, or JAK inhibitors.

An antibiotic agent is an agent that destroys or inhibits the growth of microorganisms. Non-limiting examples of antibiotic agents can include tetracycline, oxytetracy cline, metacycline, doxycycline, minocycline, erythromycin, lincomycin, penicillin G, clindamycin, kanamycin, chloramphenicol, fradiomycin, streptomycin, norfloxacin, ciprofloxacin, ofloxacin, grepafloxacin, levofloxacin, sparfloxacin, ampicillin, carbenicillin, methicillin, cephalosporins, vancomycin, bacitracin, gentamycin, fusidic acid, ciprofloxin and other quinolones, erythromycin, gentamicin, sulfonamides, trimethoprim, dapsone, isoniazid, teicoplanin, avoparcin, synercid, virginiamycin, piperacillin, ticarcillin, cefepime, cefpirome, rifampicin, pyrazinamide, enrofloxacin, amikacin, netilmycin, imipenem, meropenem, inezolidcefuroxime, ceftriaxone, cefadroxil, cefazoline, ceftazidime, cefotaxime, roxithromycin, cefaclor, cefalexin, cefoxitin, amoxicillin, co-amoxiclav, mupirocin, cloxacillin, and co-trimoxazole.

A pharmaceutical formulation can further comprise an excipient. An excipient can be a buffer, a carrier, a stabilizer, a solubilizer, a filler, a preservative, a dilutant, a vehicle, a detergent, a salt, a peptide, a surfactant, an oligosaccharide, an amino acid, an adjuvant, a carbohydrate, and/or a bulking agent.

The engineered cells as disclosed herein can be administered to the subject prior to, concurrently with, or subsequent to activation of the heterologous genetic circuit(s) in the engineered stem cells. For example, the engineered cells can be activated subsequent to being administered into the subject, e.g., by administering to the subject an activator of the heterologous genetic circuit(s).

The subject can be treated (e.g., administered with) a population of engineered cells (e.g., engineered hematopoietic progenitor cells) of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses. Alternatively, or in addition to, the subject can be treated (e.g., administered with) a population of engineered cells (e.g., engineered hematopoietic progenitor cells) of the present disclosure for 1 week, 2 weeks, 3 weeks, 4 weeks, 5 weeks, 6, weeks, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or 100 years.

Any one of the methods disclosed herein can be utilized to treat a target cell, a target tissue, a target condition, or a target disease of a subject.

Non-limiting examples of the target tissue can include cells, for example hematopoietic progenitor cell, can be obtained from a subject. Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

The target disease of the subject can be cancer or tumor. Non-limiting examples of cancer can include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Vemer Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

The present disclosure also provides a composition comprising the engineered genetic circuit(s) as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered cells. Alternatively, or in addition to, the activator(s) can be in a different and separate composition from the engineered cells.

### EXAMPLES

### Example 1: Differentiation of Hematopoietic Progenitor Cells

In this example, induced pluripotent stem cells (iPSCs) are differentiated into hematopoietic progenitor cells using various heterologous genetic circuits (HGCs).

iPSC Maintenance

Episomal iPSCs (Gibco, CatA18945) were maintained in complete Essential 8™ Flex Medium (Gibco, Cat #A2858501) or StemFlex™ Medium (Gibco, Cat #A3349401) on Vitronectin (Gibco, Cat #A31804)-coated flasks, and passaged to maintain 50-90% confluency using Versene (Gibco, Cat #15040066) to lift cells while maintaining cell clumps.

### Nucleofection

On Day 0 of the experiment, iPSC cells were collected as a single cell suspension using ACCUTASE™ (Stemcell Technologies, Cat #07920). Cells were resuspended at 1.2e9 cells/ml in P3 Primary Cell Nucleofector™ Solution and 8e5 cells were nucleofected per well in a 96 well nucleocuvette (Lonza, Cat #V4SP-3096) on Lonza's 4D Nucleofector with 96 well shuttle (Cat #AAF-1003B/S) using optimized pulse codes.

Cells were nucleofected with plasmids encoding Cas9-VPR, the core cascade and the gene-targeting spacers. Cas9-VPR is a Cas-transcriptional modulator system which can be used for both cleavage (e.g., of a gate moiety plasmid, of a gene-regulating moiety plasmid) and non-cleavage gene regulation (e.g., CRISPR activation of a target endogenous gene, CRISPR inhibition of a target endogenous gene) depending on the specific gNA used. The activatable gNAs used are polyT tract only, with a constant core cascade that executes four sequential steps. Nucleotide gNA spacers were employed to target the genome, according the sequences in Table 1. FIG. 2 describes the gene targets used in each heterologous genetic circuit cascade. The steps refer to the order in which it is expected the genes were expressed as a consequence of the Cas9-VPR-mediated targeting and activation.

TABLE 1
Spacer Sequences

| SEQ ID NO | Spacer Sequence | Spacer Gene Target |
|---|---|---|
| 1 | AAATGCACCGCTGA | BMI1 |
| 2 | CGCCCTGCTCCGCG | BMI1 |
| 3 | TGTAGTCAGGCCCG | BMI1 |
| 4 | CACGTGACCCGCTg | BMI1 |
| 5 | AGTGTGTCACACGG | ERG |
| 6 | GCATGGCTTCACAC | ERG |
| 7 | GATGATATGCAGCC | ERG |
| 8 | CAGCCCAAAGAAAC | ERG |
| 9 | AGCGACCCCGAGTT | ERG |
| 10 | GGGACCTCGGTCGT | ERG |
| 11 | CTCATCAGCATCGA | ERG |
| 12 | AGTCCGGCCCCAGC | ERG |
| 13 | GGGCGATGTCCGCT | ETS1 |
| 14 | GAAACGCCCTAAAg | ETS1 |
| 15 | CGGCCCAAAGCGAA | ETS1 |
| 16 | GGAGGAGCAGTGCG | ETS1 |
| 17 | GAGCAACACCAGCT | ETV2 |
| 18 | GCTCCAAGAGTCCC | ETV2 |
| 19 | GAACTGCGTCCCCG | ETV2 |
| 20 | GTACAGTCCGTGCG | ETV2 |
| 21 | GGTCCCATGCTTGT | GATA1 |
| 22 | CTCAGTGTGATCCC | GATA1 |
| 23 | AGACGCACATACAC | GATA1 |
| 24 | TCCAAGAATCCCCA | GATAI |
| 25 | ccgcgggTCAGTCC | GATA2 |
| 26 | GCGCCCCACCACTA | GATA2 |
| 27 | GGAGGGAACGGTCT | GATA2 |
| 28 | GCCCAGAGCATCGA | GATA2 |
| 29 | GCGTTGCGGCCGGT | GATA2 |
| 30 | CCCTGAGTGCCTAG | GATA2 |
| 31 | GTGCGCTCCGGAGA | GATA2 |
| 32 | GGGTGCAGAGCCCC | GATA2 |
| 33 | CTTCGCTAGGAAAG | GFI1 |
| 34 | CTGTAGTTCCCATC | GFI1 |
| 35 | ATGCCCAGTGGTCG | GFI1 |
| 36 | CTGGCTGTGCTTTA | GFI1 |
| 37 | ACTCAGTAACGCTC | GFI1 |
| 38 | CTGTGGGTCGGCAG | GFI1 |
| 39 | GGGACGGGCTGGAA | GFI1 |
| 40 | CAACCCCCGTCGGG | GFI1 |
| 41 | CCGGGGTCGAATTG | HOXA5 |
| 42 | ACTAATAGGGGAGT | HOXA5 |
| 43 | GCGGCAACTggcgg | HOXA5 |
| 44 | GCAGGATTTACGAC | HOXA5 |
| 45 | CCGCCTAGAATGGA | HOXA7 |
| 46 | GCCAACCTCCGCAC | HOXA7 |
| 47 | CTAGCAGGAGTCGG | HOXA7 |
| 48 | GCAGGCTGTTGCGG | HOXA7 |
| 49 | CCGCGCCTTCTTGA | HOXA9 |
| 50 | CCGCACGCTATTAA | HOXA9 |
| 51 | TGCCGCTCTACGAT | HOXA9 |
| 52 | GCACGTGACGCGCA | HOXA9 |
| 53 | AGGTTGCGACTGGA | HOXB4 |
| 54 | GAGAGAGCGAGGCA | HOXB4 |
| 55 | CAGGAGGGCTACAC | HOXB4 |
| 56 | GCGATTCCCGGATA | HOXB4 |
| 57 | CGCGGCCTTAAAAA | IRF8 |
| 58 | AGCGGCTCCAGAAG | IRF8 |
| 59 | CAGCTTGGCTCGCG | IRF8 |
| 60 | CCGCCGCACTCCCG | IRF8 |
| 61 | GCCGCGTAGAAGGA | LCOR |
| 62 | GCGGGACTGACTTC | LCOR |
| 63 | CCGTGGCCCCGGCG | LCOR |
| 64 | AGGAGCAGTTTCGA | LCOR |
| 65 | GGGTTTATCTCAGG | LCOR |
| 66 | CTGGACAACAGTCA | LCOR |
| 67 | GCAGCCTTCTGATT | LCOR |
| 68 | CCGTCAAGTGTAGA | LCOR |
| 69 | GACCTTCCCGAGAG | LMO2 |
| 70 | GCCGAGGGCAGAGA | LMO2 |
| 71 | CTTGGCTTCTTAAA | LMO2 |
| 72 | TCAAATACAATAGA | LMO2 |
| 73 | CAAAGGTGGGCTCG | MIXL1 |
| 74 | AAGCGATTATTCCC | MIXL1 |
| 75 | ATCCACATCCCCGA | MIXL1 |
| 76 | GCCATGTAAGGCAC | MIXL1 |
| 77 | GCGCCGAATGGGAG | MYB |
| 78 | CCGCGACAGTGAGT | MYB |
| 79 | ACCGCGGAGGCGGC | MYB |
| 80 | CAAACAAAGAGCTT | MYB |
| 81 | AAGGGGTCCGCACG | RUNX1 |
| 82 | CGTACAGTAGCGCG | RUNX1 |
| 83 | GTAGACTTTGCAAG | RUNX1 |
| 84 | GTAAGCCCGGCCGG | RUNX1 |
| 85 | GAACCAATTGAGAT | RUNX1 |
| 86 | GCCAGCCCGTTGTA | RUNX1 |
| 87 | GGACTTAACTCTCC | RUNX1 |
| 88 | TATCAGCCAGGGCG | RUNX1 |
| 89 | TTCTTGCGCGCCCG | SATB1 |
| 90 | AGGGCGCTGTGCGA | SATB1 |
| 91 | tggggattaaaacc | SATB1 |
| 92 | GAGGAAGGCGGCGT | SATB1 |
| 93 | GAGATAGTCCCCTT | SPI1 |
| 94 | AGGGATGACTTTGG | SPI1 |
| 95 | CTTGCGCTACATAC | SPI1 |
| 96 | CTCTGCCACACCAC | SPI1 |
| 97 | CGAGGTCACGGCGT | TBX6 |
| 98 | CCGCGTGGTCCGTC | TBX6 |
| 99 | GGGGCAGCGTCCCA | TBX6 |
| 100 | CCAGACCTTCTGGC | TBX6 |
| 101 | GCTTTCACGGAAGG | TBXT |
| 102 | CTGCGCATCGGGTG | TBXT |
| 103 | ccgcACTTCCGCGA | TBXT |
| 104 | GCGCCAAGCGGTGT | TBXT |
| 105 | CAAAGCGAGTTTCC | TAL1 |
| 106 | TAGGGAGACGAGAA | TAL1 |
| 107 | GTCCGTGTTGGGAA | TAL1 |
| 108 | CTGGAATTGGGTTC | TAL1 |
| 109 | TTACAGCGCGTCGG | TAL1 |
| 110 | GAGGCGCTTATCGG | TAL1 |
| 111 | TCGAAAGGAACCGA | TAL1 |
| 112 | GCGTACCCCCGTAG | TAL1 |

### Cell Culture

1.8e5 nucleofected cells were cultured per well in 24 well tissue culture plates treated with Anti-adherence rinsing solution (Stemcell Technologies, Cat #07010) plates rotating at 100 RPM under standard normoxia conditions in a humidified 5% CO₂ tissue culture incubator, in 500 ul per well. Day 0 media included: StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045) and 10 mM Y-27632 (Bio-Techne Cat #1254), 50 ng/ml rhBMP4 (Peprotec, Cat #120-05ET), 50 ng/ml FGF-2 (Peprotech, Cat #3718-FB) and 10 uM CHIR99021 (Bio-Techne, Cat #4423).

On Day 1, cultures underwent a complete media change with StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045), 50 ng/ml rhBMP4 (Peprotec, Cat #120-05ET), 50 ng/ml FGF-2 (Peprotech, Cat #3718-FB), and 50 ng/ml VEGF-165 (Peprotech, Cat #100-20). On Day 2, 6 uM SB431542 (Selleckchem, Cat #101762-616) is added to the cultures. On Day 4, cultures underwent a media change with StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045), 50 ng/ml FGF-2 (Peprotech, Cat #3718-FB), 50 ng/ml VEGF-165 (Peprotech, Cat #100-20) and 50 ng/ml SCF (Peprotech, Cat #300-07).

### Flow Cytometry

Cultures were analyzed on Days 3, 5 and 9 by flow cytometry on a Penteon (Agilent, Cat #2010284AA). Embryoid bodies were harvested into 96 deep-well plates (Nest, Cat #503162) and disaggregated in 500 ml TrypLE™ Express Enzyme (Gibco, Cat #12605028) at 37 C for 20-30 minutes followed by mechanical disruption by pipetting. TrypLE™ Express Enzyme is quenched with DMEM-F12 (Stemcell Technologies, Cat #36254). Cells were stained for 20 min at room temperature with Zombie Aqua™ Fixable (Biolegend, Cat #423102, 1:2000), washed with 1×DPBS, 2% FBS, 0.02% NaN3, and blocked for 10 min on ice with Human IgG (Lee Biosolutions Cat #340-21). Cells are stained on ice for 30 mins with PDGFR-BV421 BD Bioscienes Cat #562799, 1:400), CD73-BV785 (Biolegend, Cat #344028, 1:400), CD144/VE-Cadherin-PE (Biolegend, Cat #348505, 1:100), CD34-PE-Dazz (Biolegend, Cat #343534, 1:400), CD309-PE-Cy7 (Biolegend, Cat #359912, 1:100), CD43-APC (Biolegend, Cat #343206, 1:800), CD45-AF700 (Biolegend, Cat #304024, 1:200). The cells were washed with 1×DPBS, 2% FBS, 0.02% NaN3. Cells were resuspended in 1×DPBS, 2% FBS, 0.02% NaN3 for analysis. Data was analyzed using FlowJo™ v10.8 Software (BD Life Sciences). Dead cells and doublets were excluded from analysis via Zombie Aqua and doublet discrimination gates.

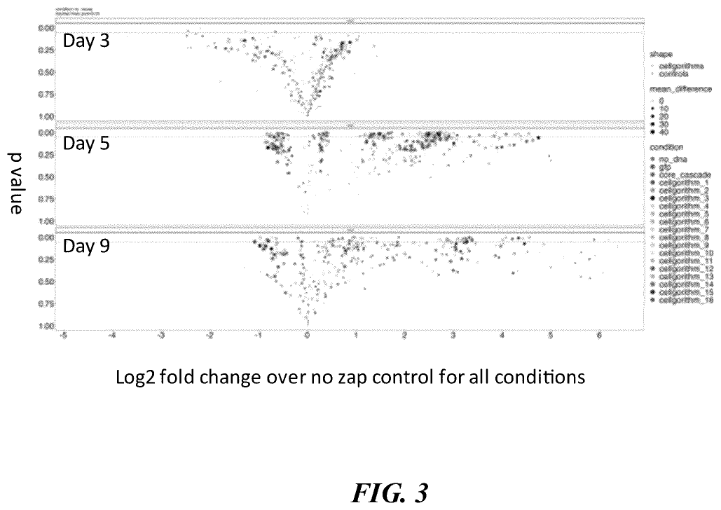

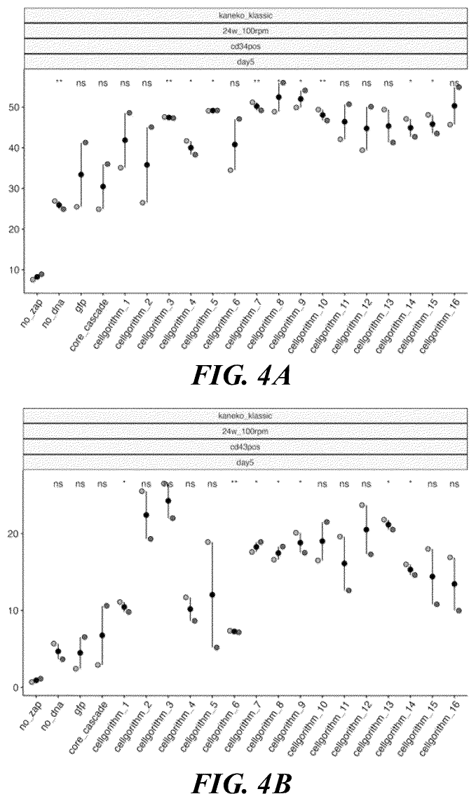

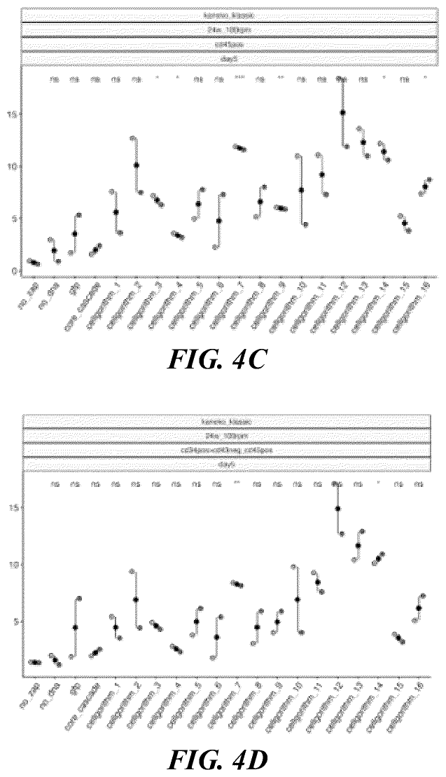

Flow cytometry data was aggregated to form FIG. 4A-4D, which depict the relative frequency of cells at day 5 of culture among appropriately sized, live, singlet cells. Scatter plots were made at varying time points to visualize the magnitude of increased hemogenic gene expression induced by the HGCs over the controls (FIG. 3).

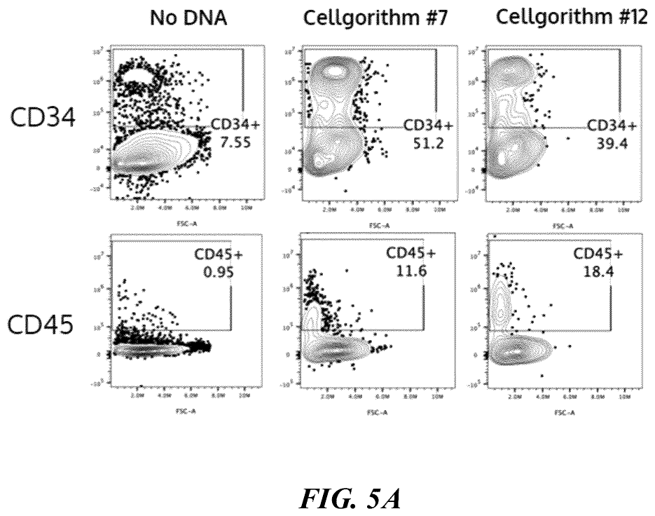

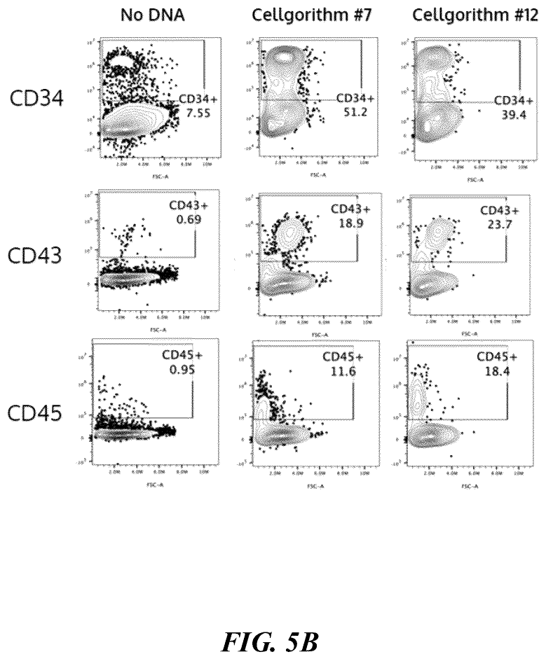

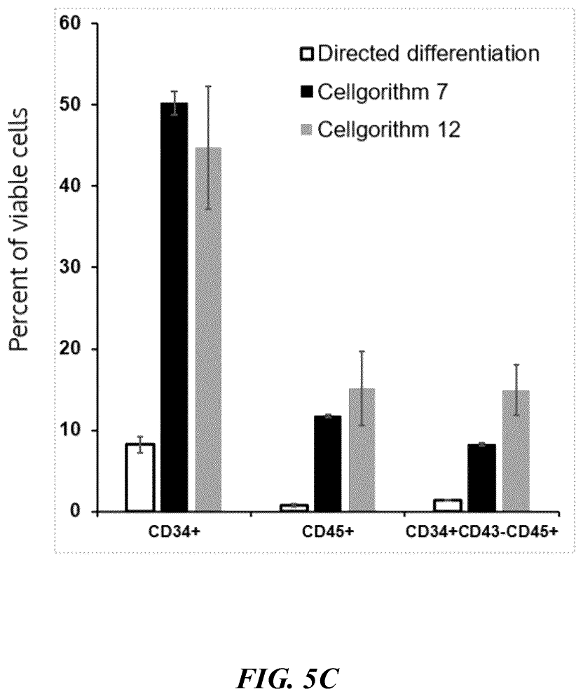

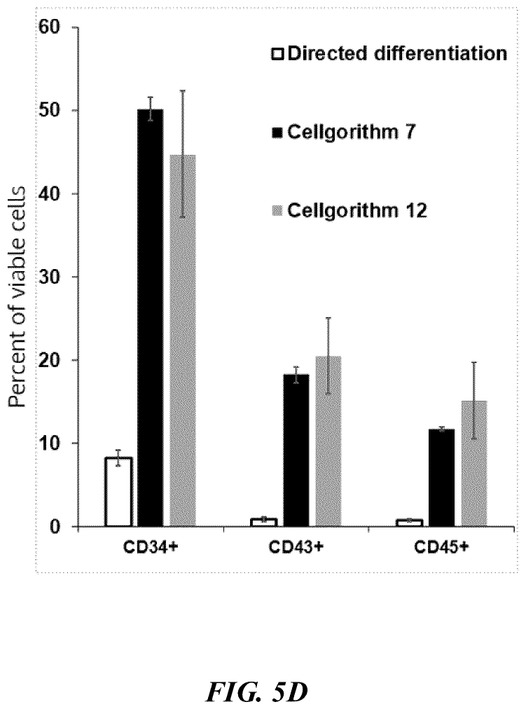

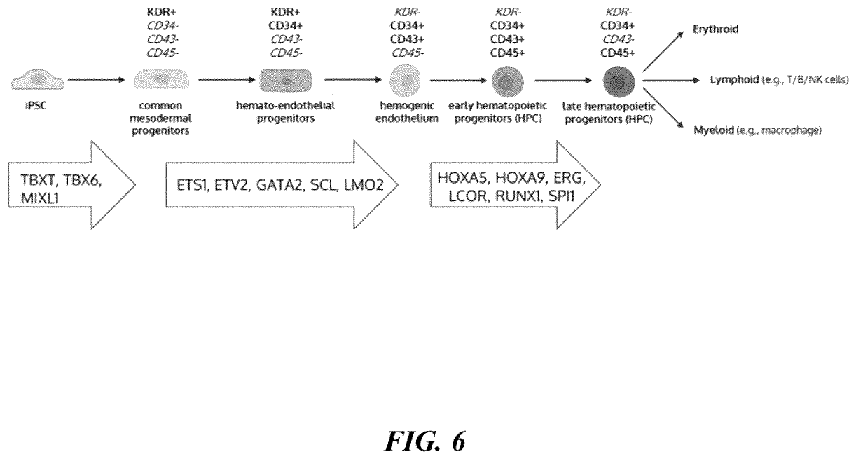

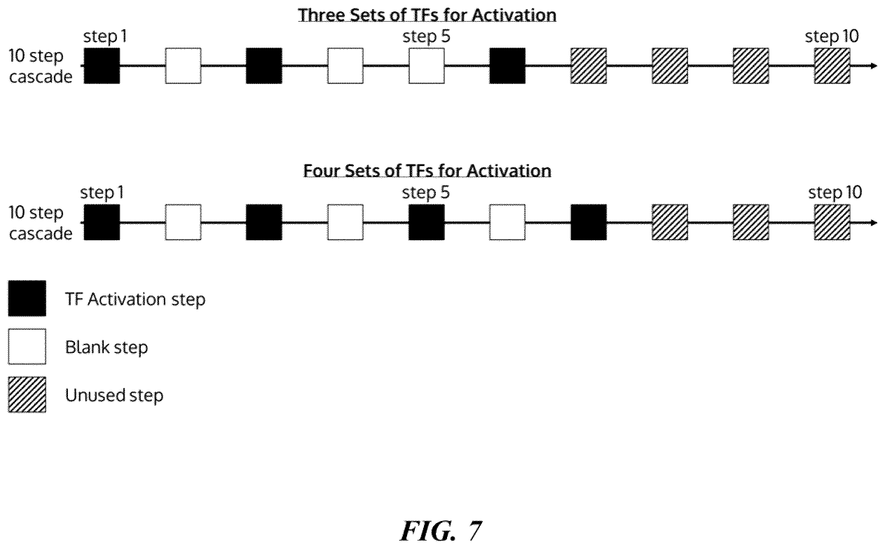

Exemplary results of flow cytometry analysis depicting the frequency of cells expressing the indicated surface marker at day 5 of culture in live singlet cells as compared to the no DNA control (e.g., which underwent nucleofection in the absence of exogenous DNA) for HSCs (Cellgorithms) #7 and #12 are shown in FIGS. 5A-5B. Summary graphs shown in FIG. 5C-5D.

### Example 2: Multi-Step Cascades

Systems and methods as provided herein (e.g., based on a polynucleotide sequence encoding an activatable sgRNA, which polynucleotide sequence comprising one or more polyT sequence) can be utilized to induce a sequentially delimited multi-step cascade effect, whereby the expression of the endogenous gene product can be activated at any step in the cascade.

For example, the multi-step cascade effect can be a 10-step cascade effect, such as a 10-step forward cascade or a 10-step reverse cascade.

### Experimental Details

In summary, the experiment involved making mixtures of plasmid DNAs encoding the components of the proGuide cascade, proceeds by introducing those DNA into cells (e.g., HEK 293 cells) via nucleofection, and concludes by evaluating the effects on activation of a target gene product at various time points using flow cytometry detection of the cell surface gene product (e.g., CXCR4).

Essential components of Cellgorithms (e.g., a Cas9-VPR expression plasmid, proGuide encoding plasmids) are mixed and a GFP expression plasmid is included to identify transfected cells. To construct combinations of plasmids to activate an endogenous gene at different steps in a cascade of proGuides, mixtures of cascade plasmid DNA used components described in Table 2 and Table 3. Core cascade plasmids are progressively included in transfection mixtures to add additional steps in a cascade as follows. For example, the first step (e.g., Step 1) condition includes no proGuides and an sgRNA with a spacer sequence targeting the 5′ and 3′ cut sites within the second step (e.g., Step 2) proGuide plasmid. The second step (e.g., Step 2) condition includes all the plasmids in the first step (e.g., Step 1) condition+proGuide plasmid described for the second step (e.g., Step 2). The third step (e.g., Step 3) condition includes all of the plasmids in the second step (e.g., Step 2) condition+the proGuide described for the third step (e.g., Step 3), and so on. To keep the mass of each proGuide plasmid DNA constant and the mass of total DNA constant for all transfections, a genetically inert plasmid DNA (e.g., pUC19) is used as a “filler” for conditions with fewer proGuide plasmids.

To activate the expression of the endogenous gene product (e.g., CXCR4), a 14 nt spacer sequence is used to target Cas9-VPR to the promoter region of the gene (e.g., CXCR4). For activation at the first step (e.g., Step 1), the gene (e.g., CXCR4) activation is stimulated by an sgRNA harboring the relevant spacer for the gene (e.g., 14 nt CXCR4 spacer). For subsequent steps, a proGuide plasmid with the relevant spacer for the gene (e.g., 14 nt CXCR4 spacer) is added to the plasmid DNA mix. By matching the 5′ and 3′ cut sites for a particular step in a cascade with the 5′ and 3′ cut sites in the gene (e.g., CXCR4)-activating proGuide, activation of the gene (e.g., CXCR4) is effectively programmed to occur at one particular step in the cascade for each condition/mixture of plasmid DNA.

Mixtures of plasmid DNA are introduced into cells (e.g., hematopoietic cells) using standard procedures with a nucleofection system (e.g., Lonza 4D). Transfected cells are plated (e.g., in multiwell tissue culture plates) and maintained using standard mammalian tissue culture methods. At specified time points (e.g., 12, 24, 36, 48 and 72 hours) after nucleofection, cells are processed for flow cytometry and detection of cell surface expression of gene product (e.g., CXCR4). For each condition, independent replicates (e.g., n=4) (nucleofections) were examined by flow cytometry.

### Results

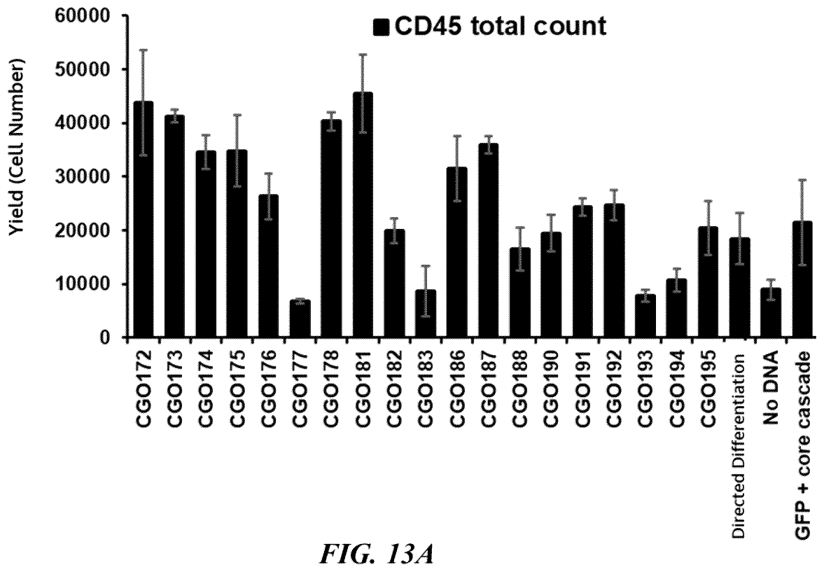

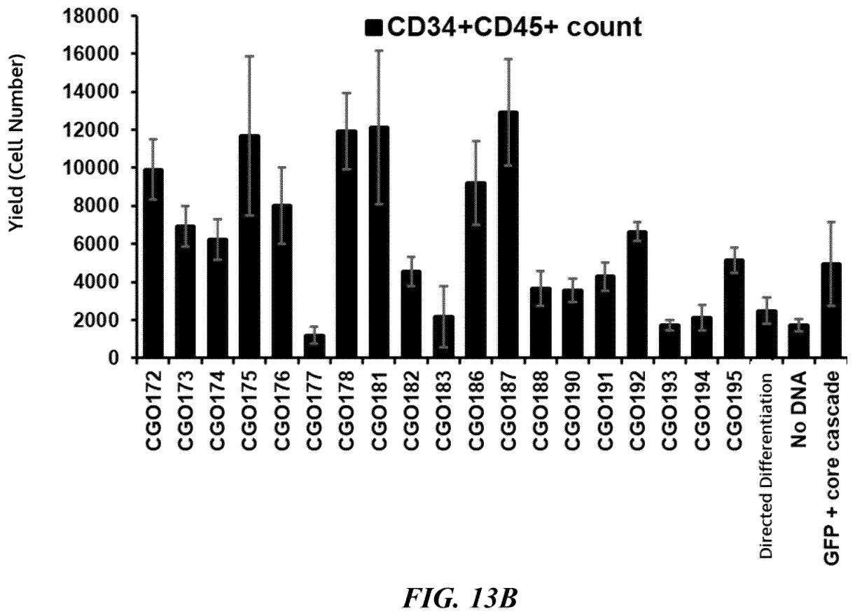

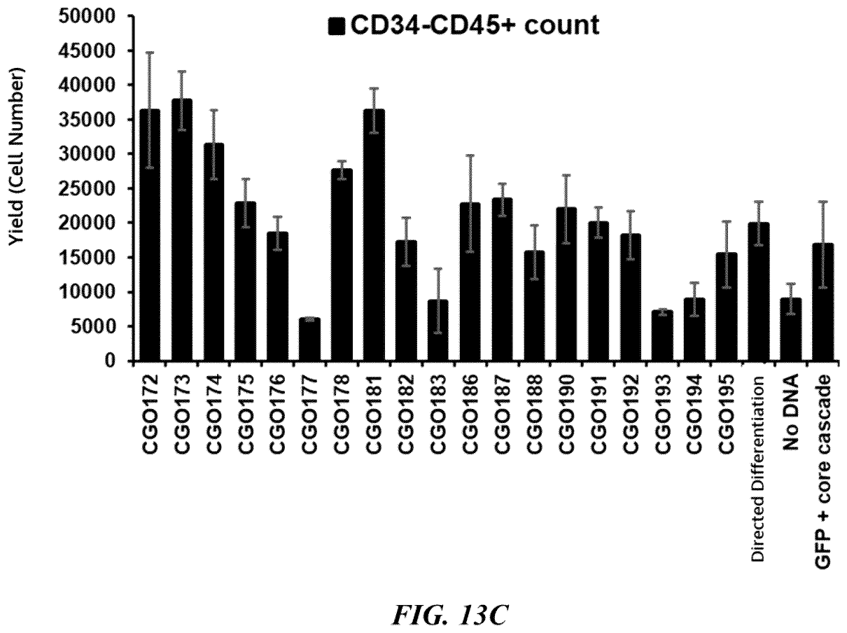

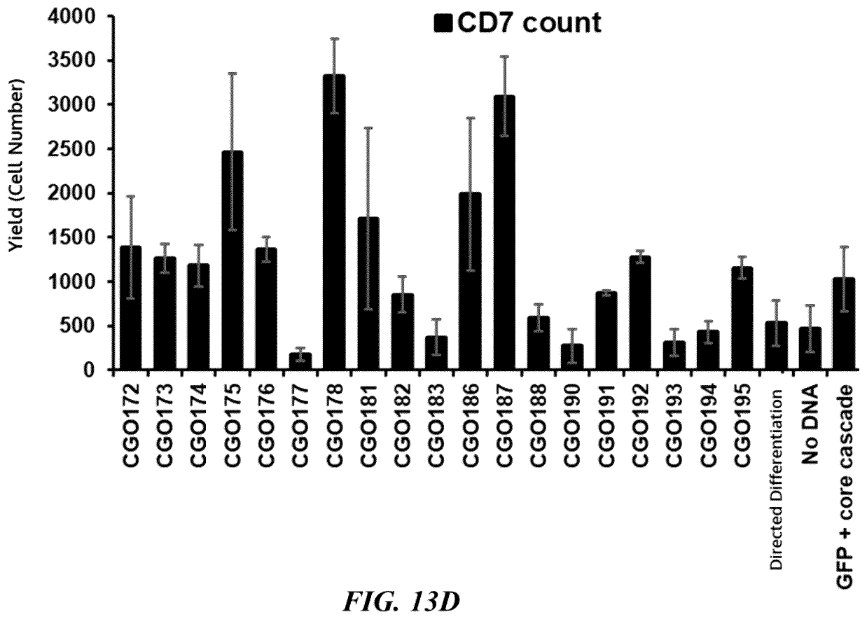

The multi-step cascade approach described above was utilized in part to generate hematopoietic progenitor cells (HPCs). The multi-step cascades produced a range of HPC expandability and yield of HPCs. HPCs derived from the multi-step cascade contain the ability to exist the HPC state and gain lymphoid potential. Some Cellgorithms promoted more loss of CD34 expression, maintain CD45 expression, and lack an increase of CD7. Some Cellgorithms enabled the HPCs to exit the progenitor state and promote an increase in yield of CD7+ cells. The CD34+CD45+ count, CD34−CD45+ count, CD45 total count, and/or CD7 count following a multi-step cascade can be found in FIGS. 13A-13D. Triple positive cell frequencies obtained in vitro were 9.18% for CD34+ cells and 8.77% for CD43+ cells.

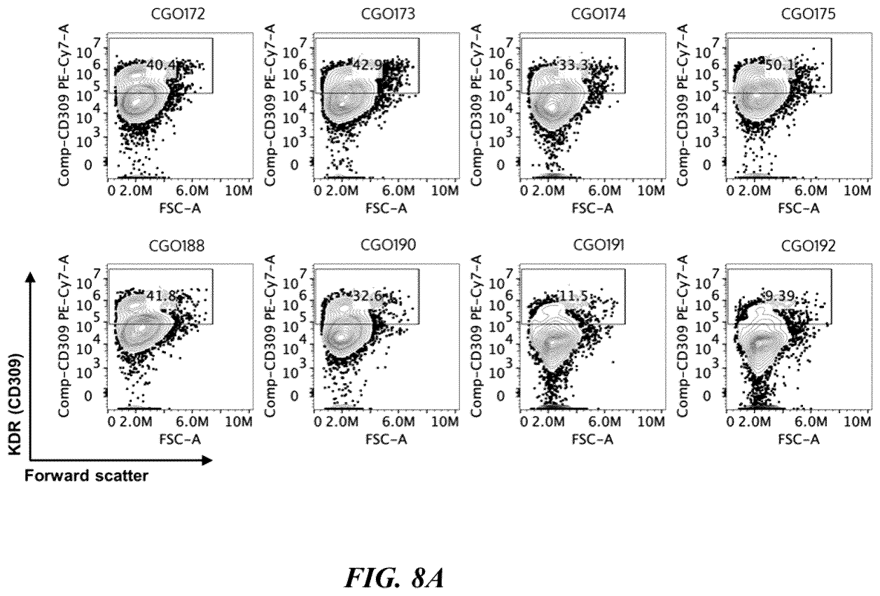

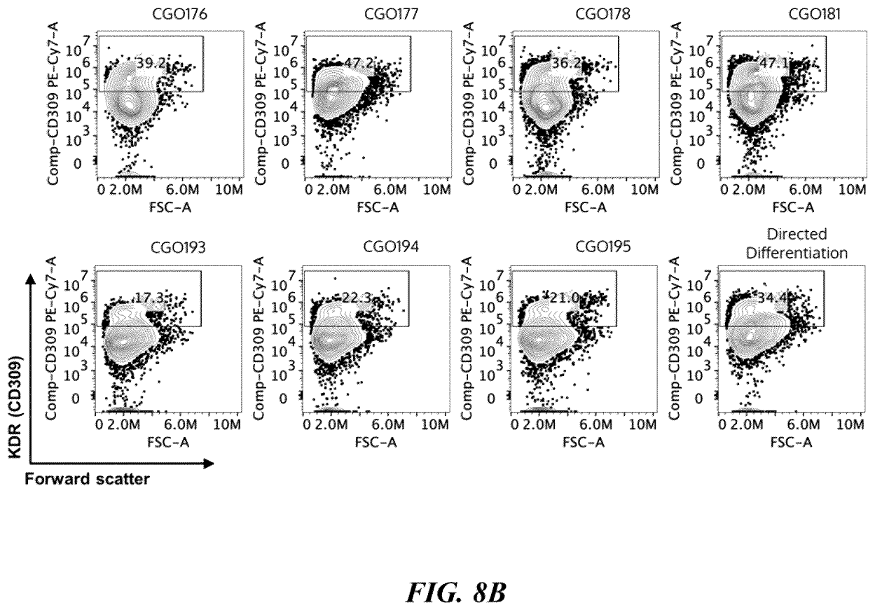

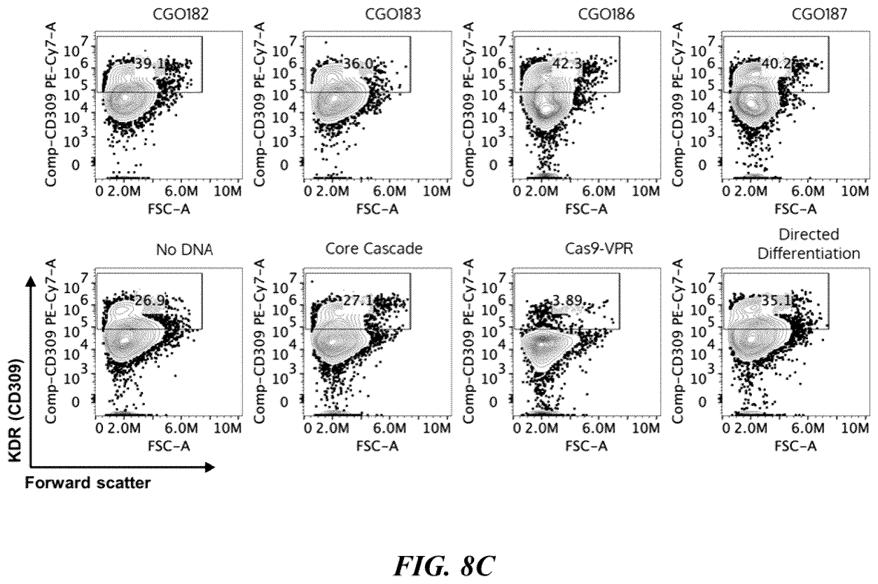

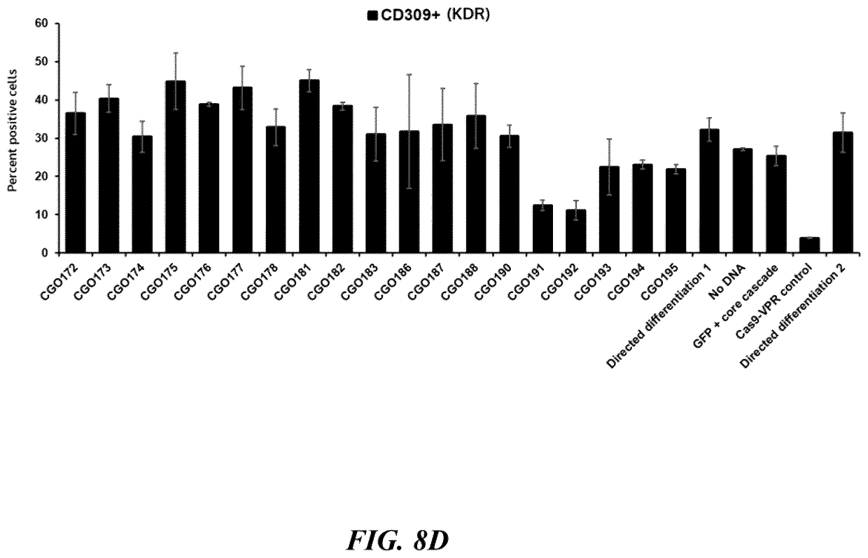

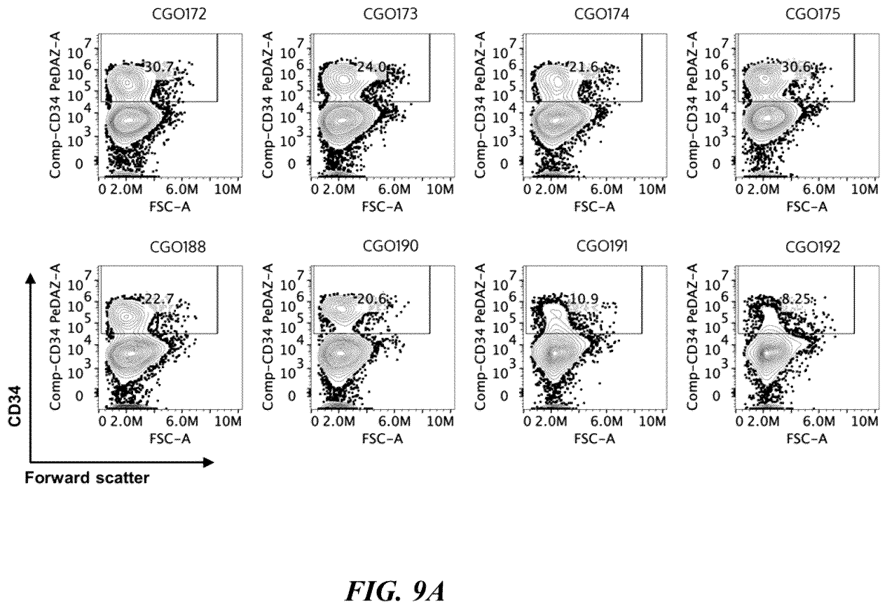

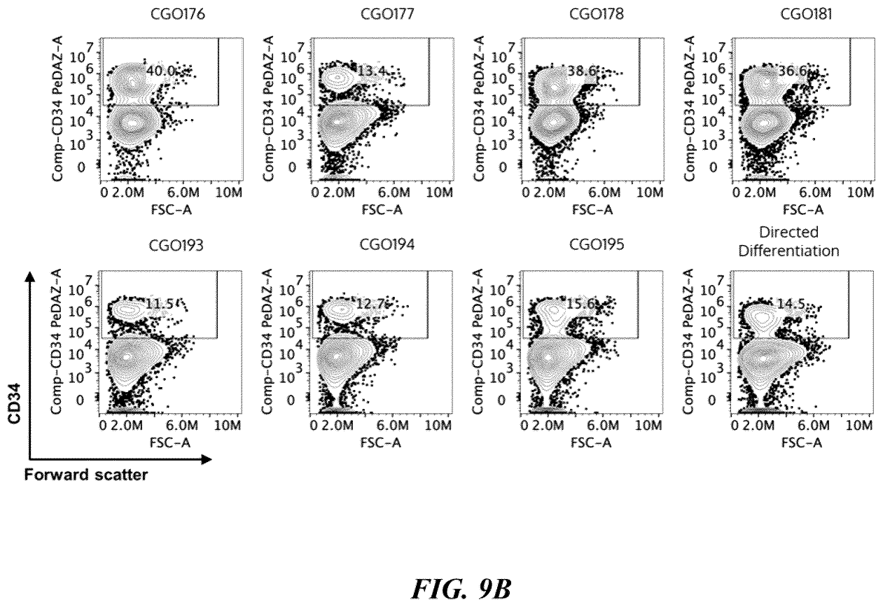

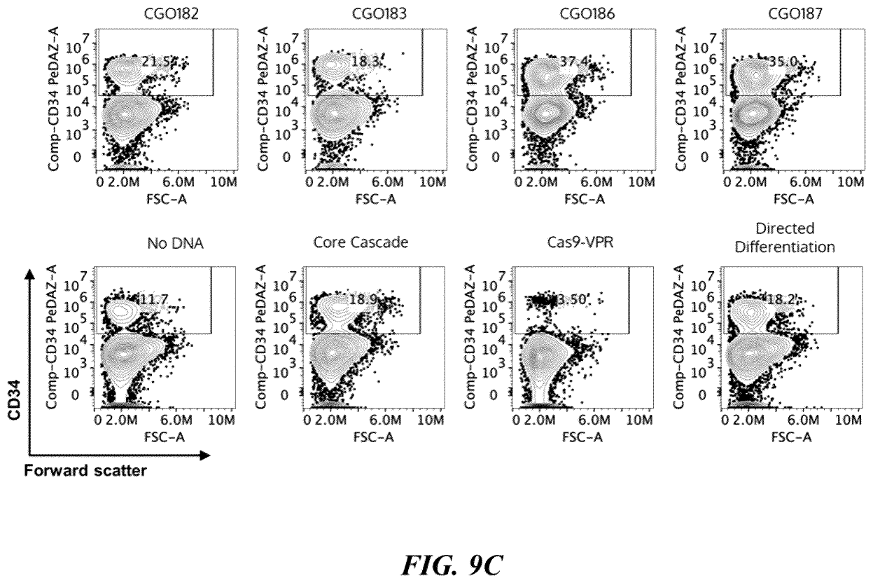

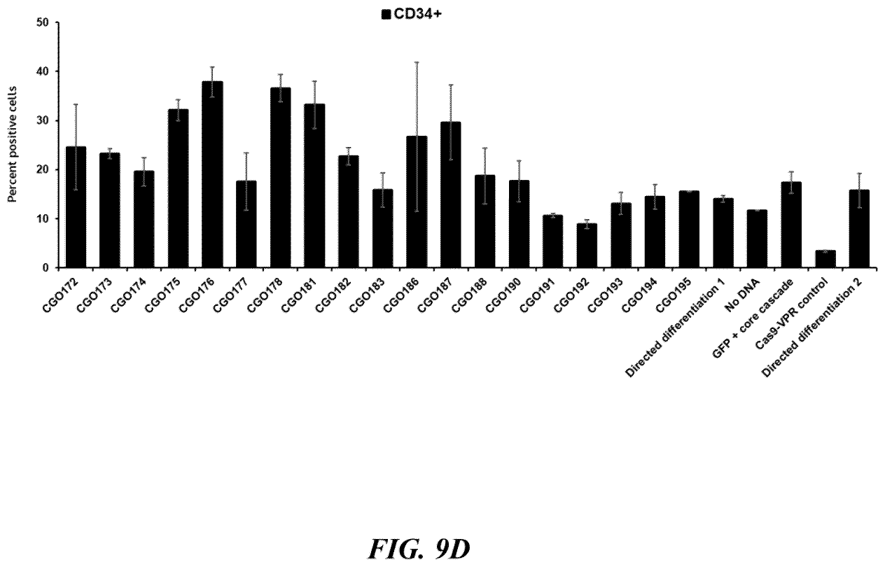

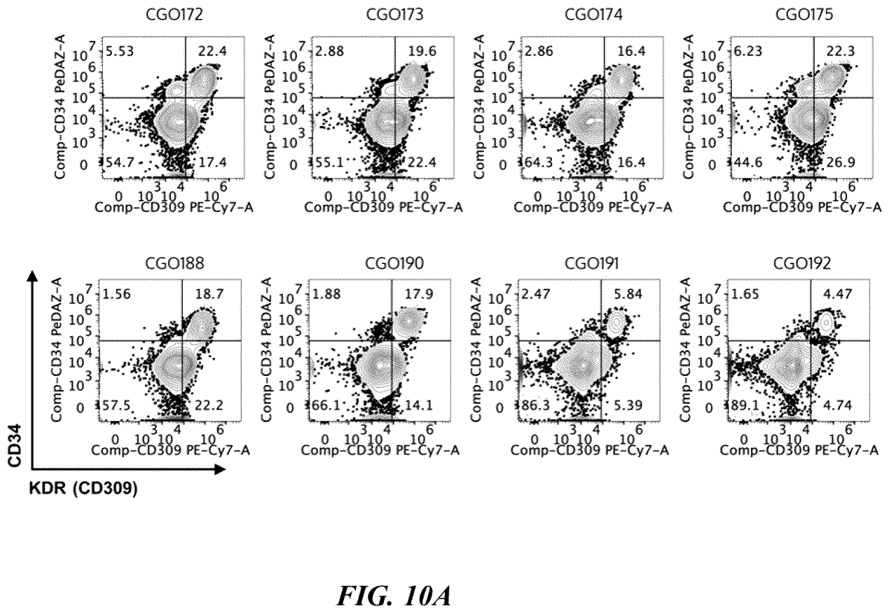

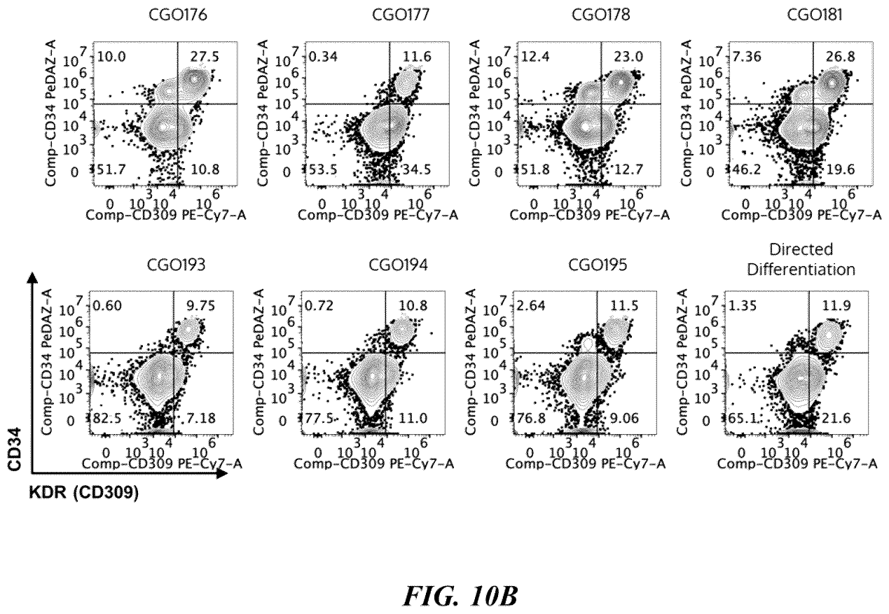

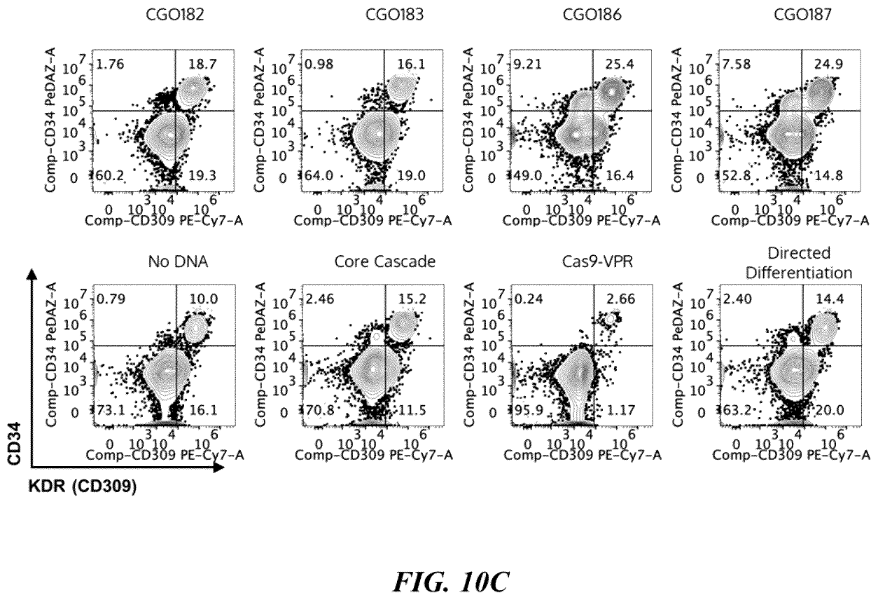

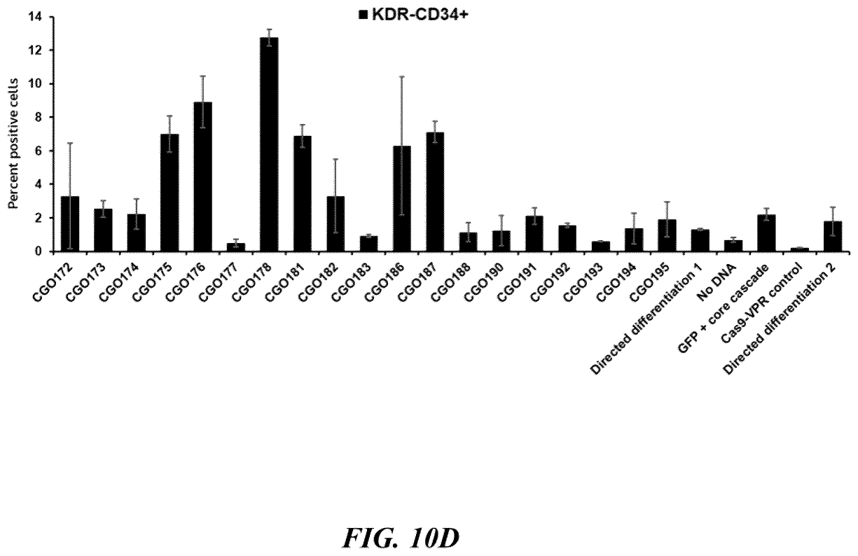

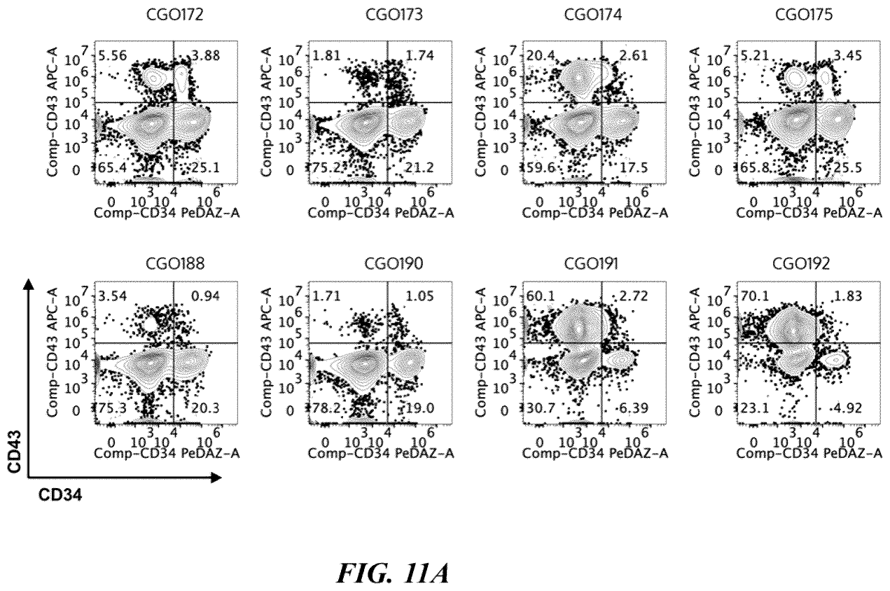

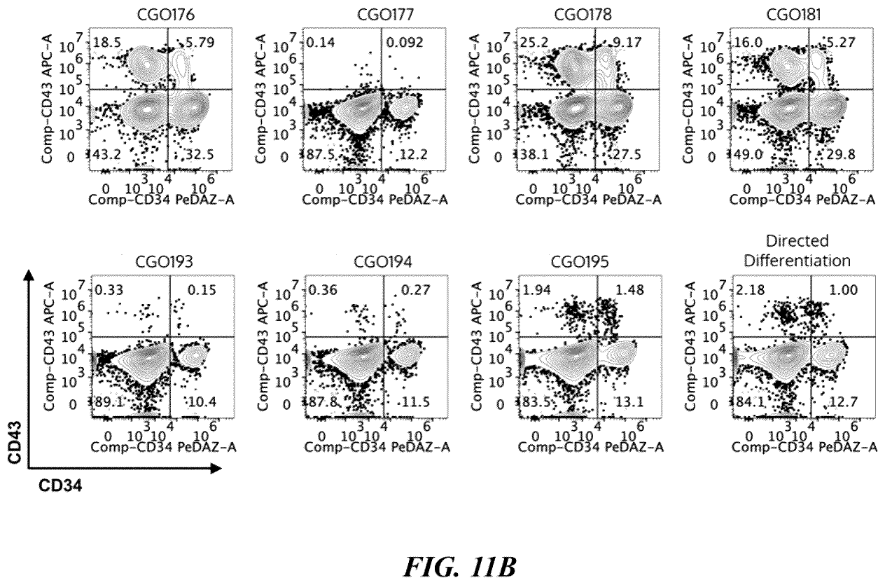

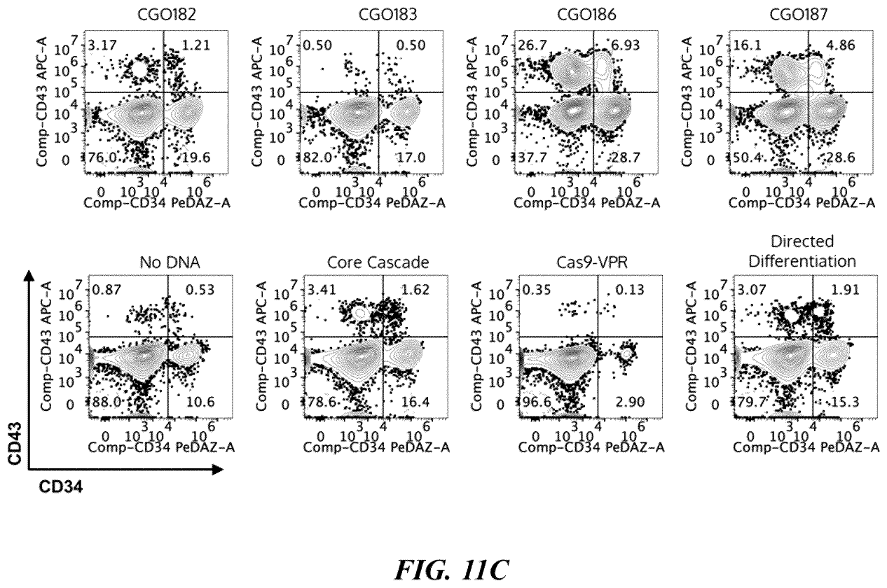

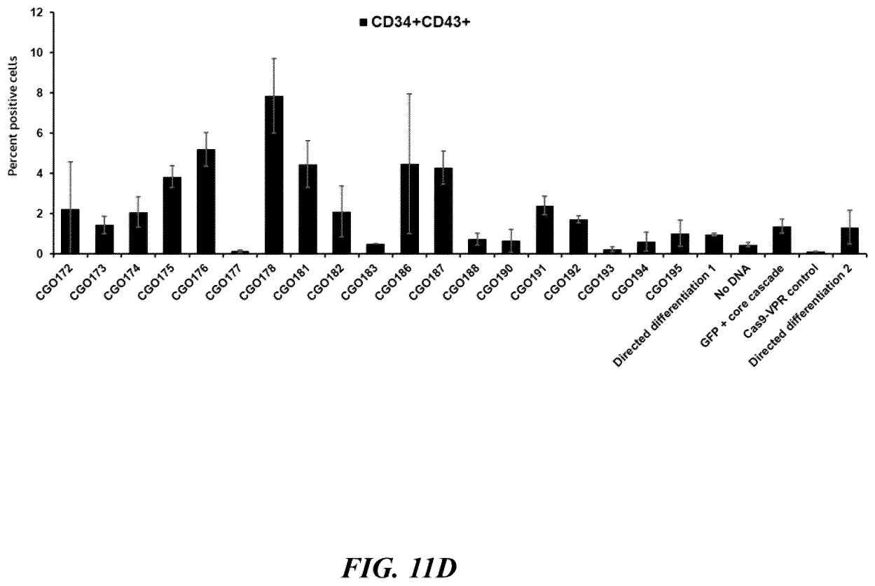

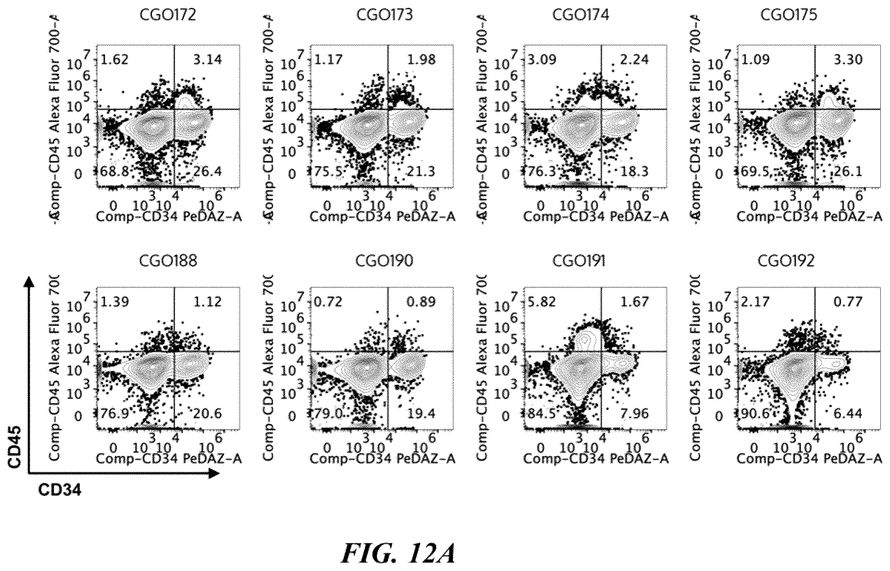

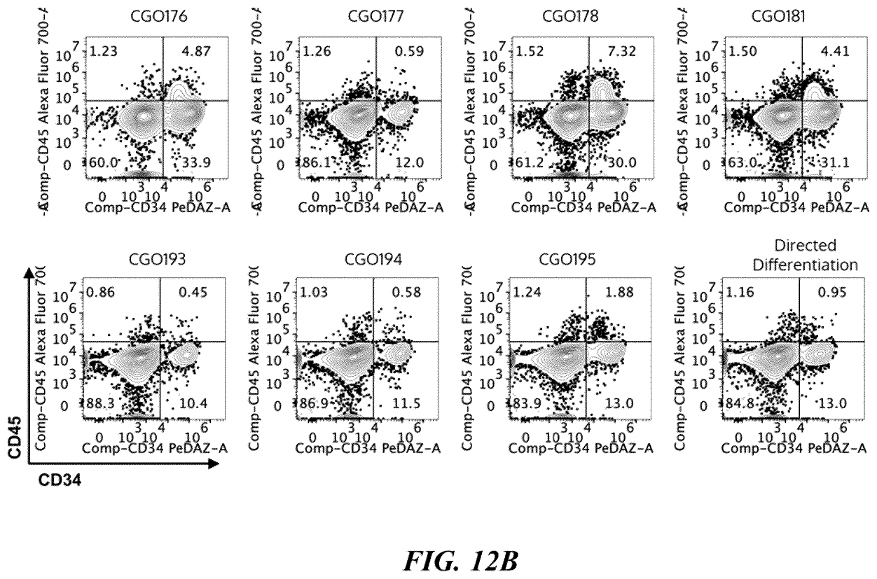

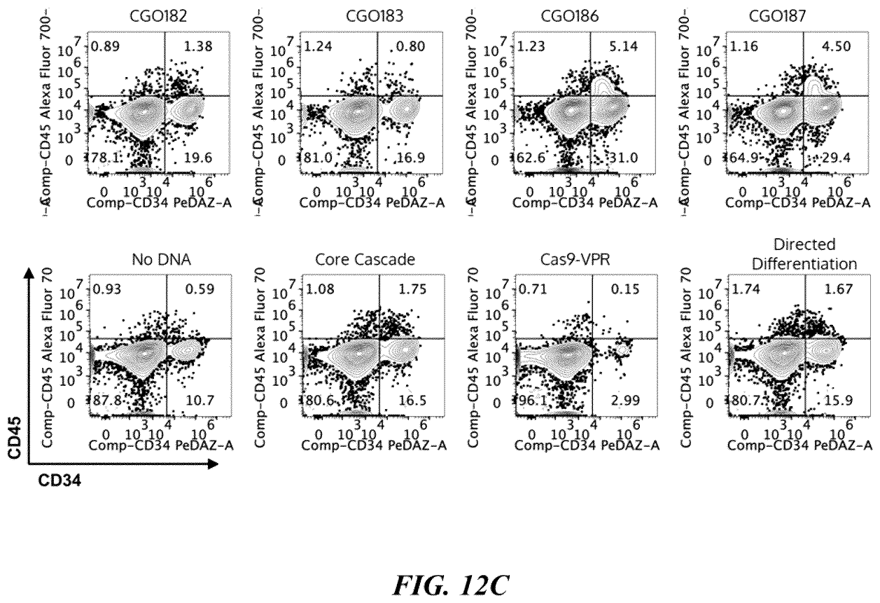

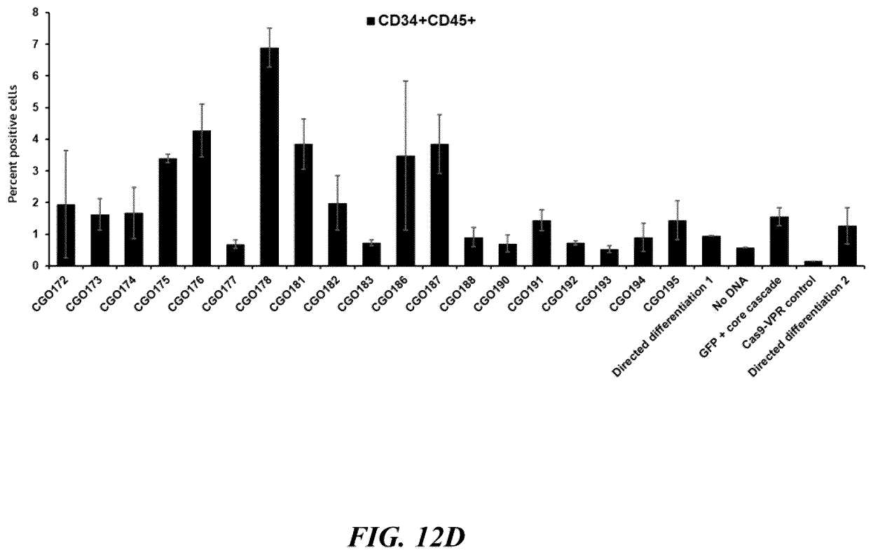

Compared to control conditions, Cellgorithms (described in Table 4) induced comparable degrees of differentiation of iPSC to early stages of the cell lineage as indicated by the lack of increase in CD309+ (KDR) cells compared to control conditions (FIGS. 8A-8D). By contrast, several, but not all, Cellgorithm treated cells displayed a significant increase in hemogenic endothelium stage (KDR− CD34+; FIGS. 9A-9D, 10A-10D) and HPC stage (CD34+ CD43+; FIGS. 11A-11D; and CD34+ CD45+; FIGS. 12A-12D). CGO178 caused the greatest increase in HPC stage cells.

Control conditions described herein include “directed differentiation” (e.g., “directed differentiation”, “directed differentiation 1”, “directed differentiation 2”) where cells were subjected to handling and addition of DNA, but not treated with electrical field pulse from nucleofection instrument (e.g., “no zap”). Control condition “no DNA” described herein is where cells were subjected to handling and electrical pulse from nucleofection instrument but no plasmid DNAs were added to cells during nucleofection. Control condition “GFP+Core Cascade” described herein is where cells were nucleofected with a GFP expression plasmid, Cas9-VPR, and the proGuides to execute progression through all steps, but no gene-targeting proGuides were included. For control condition “Cas-VPR control” described herein, cells were engineered with a control Cellgorithm genetic circuit designed to target one or more genes (e.g., CXCR4 and CD105) different from any of the genes listed in Table 4.

Both the identity of the TFs activated by Cellgorithms and their sequential order of activation were important for effects on HPC differentiation. Comparison of CGO178 vs CGO181 shows that they differ by the inclusion of 5 TFs activated in Step 6 (Table 4). The addition of these TF increased the percentage of hemogenic endothelial and HPC in cultures (FIGS. 10D, 11D, 12D). Comparison of CGO176, CGO181 and CGO192 shows that they differ by the sequential activation of the same sets of TFs (CGO176 and CGO181) versus an all-at-once activation of TFs by CGO192 (Table 4). The sequential activation of TFs by CGO176 and CGO181 produced a greater percentage of HPC cell types than the all-at-once activation by CGO192 (FIGS. 11D, 12D). For the production of hematopoietic cells, including lymphoid marker cells, similar improvements were observed from Cellgorithms with sequential activation of TFs compared to all-at-once activation and control no-activation of TFs (FIGS. 13A-13D).

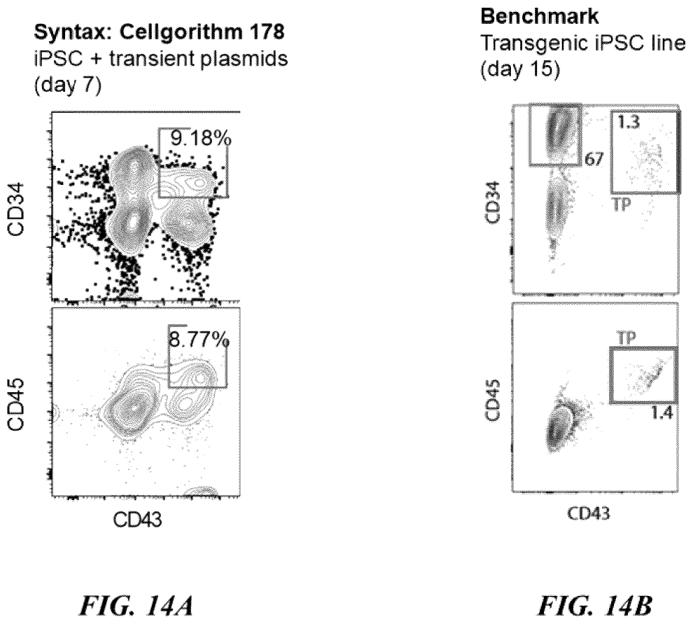

Further, CGO178 was compared against a benchmark protocol, as is described in Sugimura et al (Haematopoietic stem and progenitor cells from human pluripotent stem cells. Nature 545, 432-438 (2017).). As compared to the benchmark, the method disclosed herein yields higher conversion and faster conversion rates (FIGS. 14A-14B).

TABLE 2
Example of a heterologous genetic circuit for testing a multi-step cascade
(e.g., a 10-step forward cascade).

| | | Upstream | | Downstream cut | |
|---|---|---|---|---|---|
| Step | Stem | cut site (e.g., | | site (e.g., 3' | |
| # | name | 5' cut site) | proUnit | cut site) | Spacer sequence |
| 1 | NA | NA | NA | NA | SEQ ID NO: 172 |
| (sgRNA) | | | | | TAGCTACCGAT |
| | | | | | GTCGAGTGT |
| 2 | 1 | SEQ ID NO: 117 | SEQ ID NO: 136 | SEQ ID NO: 154 | SEQ ID NO: 173 |
| | | CCTACACTCGAC | TTTTTTTTcagc | TAGCTACCGATG | ATTACTCGAAC |
| | | ATCGGTAGCTA | caactccaaTTTTT | TCGAGTGTAGG | GTTCCGCCA |
| | | | TTT | | |
| 3 | 3 | SEQ ID NO: 118 | SEQ ID NO: 136 | SEQ ID NO: 155 | SEQ ID NO: 174 |
| | | CCCTGGCGGAA | | ATTACTCGAACG | GCGCACGACCA |
| | | CGTTCGAGTAAT | | TTCCGCCAGGG | CTATCGTGT |
| 4 | 7 | SEQ ID NO: 119 | SEQ ID NO: 136 | SEQ ID NO: 156 | SEQ ID NO: 175 |
| | | CCTACACGATAG | | GCGCACGACCA | ACTCGTTCGAT |
| | | TGGTCGTGCGC | | CTATCGTGTAGG | AGAGAGTTC |
| 5 | 6 | SEQ ID NO: 120 | SEQ ID NO: 136 | SEQ ID NO: 157 | SEQ ID NO: 176 |
| | | CCCGAACTCTCT | | ACTCGTTCGATA | CCTCCGTGTCG |
| | | ATCGAACGAGT | | GAGAGTTCGGG | ATCGTGCCA |
| 6 | 12 | SEQ ID NO: 121 | SEQ ID NO: 136 | SEQ ID NO: 158 | SEQ ID NO: 177 |
| | | CCATGGCACGAT | | CCTCCGTGTCGA | GCTCAGTCGCG |
| | | CGACACGGAGG | | TCGTGCCATGG | AATGAGCTT |
| 7 | 13 | SEQ ID NO: 122 | SEQ ID NO: 136 | SEQ ID NO: 159 | SEQ ID NO: 178 |
| | | CCAAAGCTCATT | | GCTCAGTCGCG | TAGCTCCCGTC |
| | | CGCGACTGAGC | | AATGAGCTTTGG | CGTAGACGT |
| 8 | 8 | SEQ ID NO: 123 | SEQ ID NO: 136 | SEQ ID NO: 160 | SEQ ID NO: 179 |
| | | CCGACGTCTACG | | TAGCTCCCGTCC | TGCGTCGTCTA |
| | | GACGGGAGCTA | | GTAGACGTCGG | CTACCTCTC |
| 9 | 11 | SEQ ID NO: 124 | SEQ ID NO: 136 | SEQ ID NO: 161 | SEQ ID NO: 180 |
| | | CCCGAGAGGTA | | TGCGTCGTCTAC | TGCTACGCATA |
| | | GTAGACGACGC | | TACCTCTCGGG | CGTGACGAC |
| | | A | | | |
| 10 | 10 | SEQ ID NO: 126 | SEQ ID NO: 136 | SEQ ID NO: 162 | NA(gene specific) |
| | | CCCGTCGTCAC | | TGCTACGCATAC | |
| | | GTATGCGTAGCA | | GTGACGACGGG | |

TABLE 3
Example of an additional heterologous genetic circuit for testing a multi-step
cascade (e.g., a 10-step reverse cascade, based on having the order of the
downstream/upstream cut site pairs reversed from the heterologous genetic
circuit in Table 2).

| | | Upstream cut | | Downstream cut | |
|---|---|---|---|---|---|
| | Stem | site (e.g., | | site (e.g., 3' | |
| Step # | name | 5' cut site) | proUnit | cut site) | Spacer sequence |
| 1 | NA | NA | NA | NA | SEQ ID NO: 181 |
| (sgRNA) | | | | | TGCTACGCATAC |
| | | | | | GTGACGAC |
| 2 | 10 | SEQ ID NO: 127 | SEQ ID NO: 145 | SEQ ID NO: 163 | SEQ ID NO: 182 |
| | | CCCGTCGTCACG | TTTTTTTTcagccaa | TGCTACGCATAC | TGCGTCGTCTAC |
| | | TATGCGTAGCA | ctccaaTTTTTTTT | GTGACGACGGG | TACCTCTC |
| 3 | 11 | SEQ ID NO: 128 | SEQ ID NO: 145 | SEQ ID NO: 164 | SEQ ID NO: 183 |
| | | CCCGAGAGGTAG | | TGCGTCGTCTACT | TAGCTCCCGTCC |
| | | TAGACGACGCA | | ACCTCTCGGG | GTAGACGT |
| 4 | 8 | SEQ ID NO: 129 | SEQ ID NO: 145 | SEQ ID NO: 165 | SEQ ID NO: 184 |
| | | CCGACGTCTACG | | TAGCTCCCGTCC | GCTCAGTCGCG |
| | | GACGGGAGCTA | | GTAGACGTCGG | AATGAGCTT |
| 5 | 13 | SEQ ID NO: 130 | SEQ ID NO: 145 | SEQ ID NO: 166 | SEQ ID NO: 185 |
| | | CCAAAGCTCATT | | GCTCAGTCGCGA | CCTCCGTGTCGA |
| | | CGCGACTGAGC | | ATGAGCTTTGG | TCGTGCCA |
| 6 | 12 | SEQ ID NO: 131 | SEQ ID NO: 145 | SEQ ID NO: 167 | SEQ ID |
| | | CCATGGCACGAT | | CCTCCGTGTCGA | NO: 186 |
| | | CGACACGGAGG | | TCGTGCCATGG | ACTCGTTCGATA |
| | | | | | GAGAGTTC |
| 7 | 6 | SEQ ID NO: 132 | SEQ ID NO: 145 | SEQ ID NO: 168 | SEQ ID NO: 187 |
| | | CCCGAACTCTCT | | ACTCGTTCGATA | GCGCACGACCA |
| | | ATCGAACGAGT | | GAGAGTTCGGG | CTATCGTGT |
| 8 | 7 | SEQ ID NO: 133 | SEQ ID NO: 145 | SEQ ID NO: 169 | ATTACTCGAACG |
| | | CCTACACGATAG | | GCGCACGACCAC | SEQ ID NO: 188 |
| | | TGGTCGTGCGC | | TATCGTGTAGG | TTCCGCCA |
| 9 | 3 | SEQ ID NO: 134 | SEQ ID NO: 145 | SEQ ID NO: 170 | SEQ ID NO: 189 |
| | | CCCTGGCGGAAC | | ATTACTCGAACG | TAGCTACCGATG |
| | | GTTCGAGTAAT | | TTCCGCCAGGG | TCGAGTGT |
| 10 | 1 | SEQ ID NO: 135 | SEQ ID NO: 145 | SEQ ID NO: 171 | NA(gene specific) |
| | | CCTACACTCGAC | | TAGCTACCGATGT | |
| | | ATCGGTAGCTA | | CGAGTGTAGG | |

TABLE 4
Gene Activation Programs of HPC Cellgorithms

| | | | | | | | | |
|---|---|---|---|---|---|---|---|---|
| Cellgorithm | Step 1 | Step 2 | Step 3 | Step 4 | Step 5 | Step 6 | Step 7 | Design Notes |

| | | | | | | |
|---|---|---|---|---|---|---|
| CGO172 | TBXT, | ETS1, ETV2, | MYB, | | GATA1, | Meso-HE-HSC |
| | TBX6, | GATA2, SCL, | RUNX1, | | HOXA5, | gene sets |
| | MIXL1 | LMO2 | HOXB4, | | HOXA9, | |
| | | | GFI1 | | ERG | |
| CGO173 | TBXT, | ETS1 | ETS1, | | RUNX1, | Meso-HE-HSC |
| | TBX6, | | ETV2, | | HOXA9, | gene sets |
| | MIXL1 | | GATA2, | | MYB, | |
| | | | SCL, | | GFI1, | |
| | | | LMO2 | | ERG | |
| CGO174 | ETS1, | ETS1, HOXA5, | ETS1, | | ETS1, | Meso-HE-HSC |
| | ETV2, | HOXA9, ERG, | MYB, | | BMI1, | gene sets |
| | GATA2, | SPI1 | LCOR, | | SATB1 | |
| | SCL, | | RUNX1 | | IRF8, | |
| | LMO2 | | | | HOXA7 | |
| CGO175 | TBXT, | ETS1 | ETS1 | | ETS1 | Meso - ETS1 |
| | TBX6, | | | | | only |
| | MIXL1 | | | | | |
| CGO176 | TBXT, | ETS1 | | ETS1, | | Meso-HE gene |
| | TBX6, | | | ETV2, | | sets |
| | MIXL1 | | | GATA2, | | |
| | | | | SCL, | | |
| | | | | LMO2 | | |
| CGO177 | TBXT | TBX6, MIXL1 | | ETS1, | | Meso-HE gene |
| | | | | ETV2, | | sets (comparator |
| | | | | GATA2, | | vs CGO181) |
| | | | | SCL, | | |
| | | | | LMO2 | | |
| CGO178 | TBXT, | ETS1, ETV2, | | HOXA5, | | Meso-HE-HSC |
| | TBX6, | GATA2, SCL, | | HOXA9, | | gene sets |
| | MIXL1 | LMO2 | | ERG, | | |
| | | | | LCOR, | | |
| | | | | RUNX1 | | |
| CGO181 | TBXT, | ETS1, ETV2, | | | | Meso-HE gene |
| | TBX6, | GATA2, SCL, | | | | sets |
| | MIXL1 | LMO2 | | | | |
| CGO182 | TBXT, | ETV2, | | | | No ETS1 |
| | TBX6, | GATA2, SCL, | | | | comparator vs |
| | MIXL1 | LMO2 | | | | CGO181 |
| CGO183 | ETS1, | ETS1, HOXA5, | | | | No Meso, timing |
| | ETV2, | HOXA9, ERG, | | | | comparator vs |
| | GATA2, | SPI1 | | | | CGO178 |
| | SCL, | | | | | |
| | LMO2 | | | | | |
| CGO186 | TBXT, | | | ETS1, | | No Step3 ETS1 |
| | TBX6, | | | ETV2, | | comparator vs |
| | MIXL1 | | | GATA2, | | CGO176 |
| | | | | SCL, | | |
| | | | | LMO2 | | |
| CGO187 | TBXT, | | | ETV2, | | No ETS1 |
| | TBX6, | | | GATA2, | | comparator vs |
| | MIXL1 | | | SCL, | | CGO176, 186 |
| | | | | LMO2 | | |
| CGO188 | ETS1, | | | ETS1, | | No Meso, timing |
| | ETV2, | | | HOXA5, | | comparator vs |
| | GATA2, | | | HOXA9, | | CGO178 |
| | SCL, | | | ERG, | | |
| | LMO2 | | | SPI1 | | |
| CGO190 | TBXT, | | | | | Meso gene |
| | TBX6, | | | | | activation only |
| | MIXL1 | | | | | |
| CGO191 | ETS1, | | | | | Timing control |
| | ETV2, | | | | | for |
| | GATA2, | | | | | CGO193, 194, 195 |
| | SCL, | | | | | |
| | LMO2 | | | | | |
| CGO192 | ETS1, | | | | | All at once |
| | ETV2, | | | | | control for |
| | GATA2, | | | | | CGO176, 177, 181, |
| | SCL, | | | | | 186 |
| | LMO2, | | | | | |
| | TBXT, | | | | | |
| | TBX6, | | | | | |
| | MIXL1 | | | | | |
| CGO193 | | ETS1, ETV2, | | | | CGO193-195 is a |
| | | GATA2, SCL, | | | | series of 3 testing |
| | | LMO2 | | | | effects of shifting |
| CGO194 | | | | ETS1, | | activation timing |
| | | | | ETV2, | | of HE gene set to |
| | | | | GATA2, | | different steps |
| | | | | SCL, | | without previous |
| | | | | LMO2 | | Meso gene |
| CGO195 | | | ETS1, | | | activation |
| | | | ETV2, | | | |
| | | | GATA2, | | | |
| | | | SCL, | | | |
| | | | LMO2 | | | |

| | |
|---|---|
| Control | “Directed differentiation” - Cell were subjected to handling and addition of DNA, |
| | but they were not treated with electrical field pulse from nucleofection |
| | instrument aka “No Zap” |
| Control | “No DNA” - Cells were subjected to handling and electrical field pulse from |
| | nucleofection instrument, but no plasmid DNAs were added to cells during |
| | nucleofection |
| Control | “GFP + Core Cascade” - Cells were nucleofected with a GFP expression plasmid, |
| | Cas9-VPR, and the proGuides to execute progression through all steps, but no |
| | gene-targeting proGuides were included |

TABLE 5
Spacer sequences for target gene activation

| | | SEQ |
|---|---|---|
| Gene | | ID |
| Target | Spacer | NO |
| BCL11A | GCGTGTGGACGCCA | 190 |
| BCL11A | TGCAAGTTCAAGTG | 191 |
| BCL11B | GCCCGCACCGACCC | 192 |
| BCL11B | AGTCGGAGCACTGG | 193 |
| BCL11B | TCTCGGTCTCTCTA | 194 |
| BCL2 | CACAGGAAACCGGT | 195 |
| BCL2 | GCGCGTCCCGCCGG | 196 |
| BCL2 | GCGTGCGGTTCCCC | 197 |
| BCL2 | GGGTGGCTCAGAGG | 198 |
| BCL2L1 | CGGGAGAGTACTCC | 199 |
| BCL2L1 | GCCGCCCTCGATCC | 200 |
| BCL2L1 | CTCCACCTCACCCT | 201 |
| BCL2L1 | GCCGGTAACTCAGC | 202 |
| BCL2L1 | CGGACGGATGAAAT | 203 |
| BCL2L1 | TCGCGGTAGATCTG | 204 |
| BCL2L1 | GCCCGAGACGCAAA | 205 |
| BCL2L1 | tagctcaacgagag | 206 |
| BMI1 | AAATGCACCGCTGA | 207 |
| BMI1 | CGCCCTGCTCCGCG | 208 |
| BMI1 | TGTAGTCAGGCCCG | 209 |
| BMI1 | CACGTGACCCGCTg | 210 |
| CD34 | GCATCCTTCCCGCG | 211 |
| CD34 | GGTCCCAAAGGCGG | 212 |
| CD34 | AAGACTAAAAAGGG | 213 |
| CD34 | ATATGGAAGGTCAC | 214 |
| DLL4 | CCGACTGGCTGACG | 215 |
| DLL4 | GATGGCGGGGTTAA | 216 |
| DLL4 | AGCTAGAGGCCGGG | 217 |
| DLL4 | GCTGGGGGACCTAG | 218 |
| DNMT3B | GGAGACACACACAC | 219 |
| DNMT3B | GCTTCTGATCCCCC | 220 |
| DNMT3B | TGTTCCCCCGCCAT | 221 |
| DNMT3B | AGGCAGGTCCTAAA | 222 |
| DNMT3B | ACCAGGCATCTCAA | 223 |
| DNMT3B | TAAGAATGCATCCT | 224 |
| DTX1 | CCGCGAGATCCCGG | 225 |
| DTX1 | TGTTTAGGAGTCAC | 226 |
| DTX1 | CCCGAGACCCCAAG | 227 |
| EBF1 | TCAACTCGCGCTGC | 228 |
| EBF1 | TCATCTACACGCAA | 229 |
| EBF1 | TCCCGGGCCGCGAT | 230 |
| EOMES | GGAGACCCTCCTAT | 231 |
| EOMES | AGGCGCGGGCTAGT | 232 |
| EOMES | GAGCAAGAGGTACG | 233 |
| ERG | AGTGTGTCACACGG | 234 |
| ERG | GCATGGCTTCACAC | 235 |
| ERG | GATGATATGCAGCC | 236 |
| ERG | CAGCCCAAAGAAAC | 237 |
| ERG | AGCGACCCCGAGTT | 238 |
| ERG | GGGACCTCGGTCGT | 239 |
| ERG | CTCATCAGCATCGA | 240 |
| ERG | AGTCCGGCCCCAGC | 241 |
| ETS1 | GGGCGATGTCCGCT | 242 |
| ETS1 | GAAACGCCCTAAAg | 243 |
| ETS1 | CGGCCCAAAGCGAA | 244 |
| ETS1 | GGAGGAGCAGTGCG | 245 |
| ETV2 | GAGCAACACCAGCT | 246 |
| ETV2 | GCTCCAAGAGTCCC | 247 |
| ETV2 | GAACTGCGTCCCCG | 248 |
| EZH1 | ATGCGTCCTAGCAG | 249 |
| EZH1 | GTAGTGCGTCCGCG | 250 |
| EZH1 | CTGGAGAACTGGTA | 251 |
| EZH1 | GGCGCTGGCAGGGG | 252 |
| FLT1 | CTGGTGACGTCAAG | 253 |
| FLT1 | CCCGAAACTGGGGA | 254 |
| FLT1 | AGGGAAGCGAGCCT | 255 |
| FLT1 | GGGCGGGTGGCCCG | 256 |
| FOS | GGGCTCAACCACGG | 257 |
| FOS | ATGCTCACGAGATT | 258 |
| FOS | GCAGCCAACACCGA | 259 |
| FOS | ACAGGGAAAGGCCG | 260 |
| FOXO1 | GGCCAATGGGCATG | 261 |
| FOXO1 | TCCCGTAAGTCGGG | 262 |
| FOXO1 | CAGAGCCGAGTACT | 263 |
| FOXO1 | TCCCCGTGGAAAAC | 264 |
| FOXP3 | CACACCGTACAGCG | 265 |
| FOXP3 | ACAGGGCCAACCCG | 266 |
| FOXP3 | CTCTATGTGTGGAT | 267 |
| GATA1 | GGTCCCATGCTTGT | 268 |
| GATA1 | CTCAGTGTGATCCC | 269 |
| GATA1 | AGACGCACATACAC | 270 |
| GATA1 | TCCAAGAATCCCCA | 271 |
| GATA2 | ccgcgggTCAGTCC | 272 |
| GATA2 | GCGCCCCACCACTA | 273 |
| GATA2 | GGAGGGAACGGTCT | 274 |
| GATA2 | GCCCAGAGCATCGA | 275 |
| GATA2 | GCGTTGCGGCCGGT | 276 |
| GATA2 | CCCTGAGTGCCTAG | 277 |
| GATA2 | GTGCGCTCCGGAGA | 278 |
| GATA3 | GCGGCCACATTTAA | 279 |
| GATA3 | CCCAAACCCGCTCC | 280 |
| GATA3 | TTCCGGTCAGTGGA | 281 |
| GATA3 | CCTCCCCCCCGGCG | 282 |
| GFI1 | CTTCGCTAGGAAAG | 283 |
| GFI1 | CTGTAGTTCCCATC | 284 |
| GFI1 | ATGCCCAGTGGTCG | 285 |
| GFI1 | CTGGCTGTGCTTTA | 286 |
| GFI1 | ACTCAGTAACGCTC | 287 |
| GFI1 | CTGTGGGTCGGCAG | 288 |
| GFI1 | GGGACGGGCTGGAA | 289 |
| GFI1B | CGCTCCAAGTGTCG | 290 |
| GFI1B | CTCGACACTTGGAG | 291 |
| GFI1B | AATGAAGAGAGCGC | 292 |
| GFI1B | TGAAAATCAGGGAG | 293 |
| HES1 | CCTCTATATATATC | 294 |
| HES1 | CCAGGCACAAGGTC | 295 |
| HES1 | CTGAAAGTTACTGT | 296 |
| HES1 | CAACGTCAATCAAA | 297 |
| HHEX | GCGCGTCCCGGGGG | 298 |
| HHEX | GCGGAGCCTATCGC | 299 |
| HHEX | CTGGTGCCCCGCCG | 300 |
| HHEX | CGGCCAATGGCGCG | 301 |
| HOXA10 | CCAACGTGGCTGGT | 302 |
| HOXA10 | ATTTATATCAATCG | 303 |
| HOXA10 | CAAGGGCCCTTCTA | 304 |
| HOXA5 | CCGGGGTCGAATTG | 305 |
| HOXA5 | ACTAATAGGGGAGT | 306 |
| HOXA5 | GCGGCAACTggcgg | 307 |
| HOXA7 | CCGCCTAGAATGGA | 308 |
| HOXA7 | GCCAACCTCCGCAC | 309 |
| HOXA7 | CTAGCAGGAGTCGG | 310 |
| HOXA7 | GCAGGCTGTTGCGG | 311 |
| HOXA9 | CCGCGCCTTCTTGA | 312 |
| HOXB4 | AGGTTGCGACTGGA | 313 |
| HOXB4 | GAGAGAGCGAGGCA | 314 |
| HOXB4 | CAGGAGGGCTACAC | 315 |
| ID2 | GGCGCATCACGCGG | 316 |
| ID2 | GAGTGATCCCTGAC | 317 |
| ID2 | CGGAGCAGACTCTC | 318 |
| ID2 | ACATCTGGCGCAAT | 319 |
| ID2 | AGCCAATCCCCGCG | 320 |
| ID2 | AGCCCGAGCCCGGC | 321 |
| ID2 | GTACAGTAAGTgcg | 322 |
| ID3 | GCGCACGCTCGCCG | 323 |
| ID3 | CAGTGTTGGGCTAA | 324 |
| ID3 | GGAAGCGCTGATAC | 325 |
| IKZF1 | cgcgcgtaacccgt | 326 |
| IKZF1 | CGCTCCCGGCCGAC | 327 |
| IKZF1 | ACGCAAGGGCGCGG | 328 |
| IKZF1 | cgccgcatcccgtg | 329 |
| IKZF2 | GGGCTCCACCGGAT | 330 |
| IKZF2 | GAGTGCGCGCGTCG | 331 |
| IKZF2 | CGCGGGCGGAGGGC | 332 |
| IKZF2 | AAGTGCTGCCCCGG | 333 |
| IKZF2 | CACCACGTACTTTC | 334 |
| IKZF2 | GATTCTTAAGGAAC | 335 |
| IKZF2 | GGCAGAACCCAGCT | 336 |
| IL7R | CTATCTTAGGCTAG | 337 |
| IL7R | CTAACCACAGACAA | 338 |
| IL7R | AGGGAATATCCAGG | 339 |
| IL7R | GTAGATTCACACCT | 340 |
| IRF4 | GGCAAGGCCATCAC | 341 |
| IRF4 | CGGAGGGTCGCCAA | 342 |
| IRF4 | TCGGGGACTGTCAC | 343 |
| IRF4 | GCCTCGTGGCTGAA | 344 |
| IRF4 | GGCATGAACCTGGA | 345 |
| IRF4 | GCAGGGGATCGGGG | 346 |
| IRF4 | GGACGACCCTGACA | 347 |
| IRF8 | CGCGGCCTTAAAAA | 348 |
| IRF8 | AGCGGCTCCAGAAG | 349 |
| IRF8 | CAGCTTGGCTCGCG | 350 |
| IRF8 | CCGCCGCACTCCCG | 351 |
| KDR | GGTACCCGGGTGAG | 352 |
| KDR | CAGTCCAGTTGTGT | 353 |
| KDR | GCGCGTCAAAGTGC | 354 |
| KDR | GTTTGGAGCCACAA | 355 |
| LCOR | GCCGCGTAGAAGGA | 356 |
| LCOR | GCGGGACTGACTTC | 357 |
| LCOR | CCGTGGCCCCGGCG | 358 |
| LCOR | AGGAGCAGTTTCGA | 359 |
| LCOR | GGGTTTATCTCAGG | 360 |
| LCOR | CTGGACAACAGTCA | 361 |
| LCOR | GCAGCCTTCTGATT | 362 |
| LCOR | CCGTCAAGTGTAGA | 363 |
| LEF1 | AGTGTCGGGTATCA | 364 |
| LEF1 | CTCTGGAGGAAATC | 365 |
| LEF1 | AGGGCCGCGCTGGA | 366 |
| LEF1 | CCAAAGAAACTTGG | 367 |
| LEF1 | GAAGCGACGCAAGT | 368 |
| LEF1 | AGCGTGCCTTTCGG | 369 |
| LEF1 | GGCTGCCCGCTGGA | 370 |
| LEF1 | AAGACTCGTCCTAC | 371 |
| LMO2 | GACCTTCCCGAGAG | 372 |
| LMO2 | GCCGAGGGCAGAGA | 373 |
| LMO2 | CTTGGCTTCTTAAA | 374 |
| LYL1 | CAACGGGCCGGAAA | 375 |
| LYL1 | AGTCCTGCCAGCGC | 376 |
| LYL1 | CCGGAGGAAACCAG | 377 |
| LYL1 | CGGGGTCCCTACAG | 378 |
| MEEF2C | CGCGCGCGAATGCG | 379 |
| MEF2C | AGAAAACTGCGTCC | 380 |
| MEF2C | cttccGAGCGGCCT | 381 |
| MEF2C | GGTTTGGGATTGTG | 382 |
| MIXL1 | CAAAGGTGGGCTCG | 383 |
| MIXL1 | AAGCGATTATTCCC | 384 |
| MIXL1 | ATCCACATCCCCGA | 385 |
| MIXL1 | GCCATGTAAGGCAC | 386 |
| MYB | GCGCCGAATGGGAG | 387 |
| MYB | CCGCGACAGTGAGT | 388 |
| MYB | ACCGCGGAGGCGGC | 389 |
| MYC | GGAGGGGCGCTTAT | 390 |
| MYC | CTGAGTATAAAAGC | 391 |
| MYC | TTGGCAGCAAATTG | 392 |
| MYC | TGCGAGGGTCTGGA | 393 |
| NFIL3 | CGTAGCGCGGCGCT | 394 |
| NFIL3 | CGCCGCCGCGGAGG | 395 |
| NFIL3 | CCGAGCGCCCGCGA | 396 |
| NFIL3 | CGGCCATTGGCGGC | 397 |
| NR4A1 | GTTCCATTGACGCA | 398 |
| NR4A1 | CTTTGGCCATACAA | 399 |
| NR4A1 | AATAACCAGCGGGA | 400 |
| NR4A1 | TGCGGGGAGCCTAG | 401 |
| PAX5 | GGGCGCCGCCCTAG | 402 |
| PAX5 | TCTGCGCTGTGCGC | 403 |
| PAX5 | CTGCCCCTTCCCGT | 404 |
| PAX5 | CCGGAGCGAGGAAA | 405 |
| PGF | GGGAACCTCGTCTG | 406 |
| PGF | TGAGAGCCGAAGCA | 407 |
| PGF | GCAGTGCGTGCGAG | 408 |
| PGF | AGGAAGGGCCGTCC | 409 |
| RORC | ACGCCCCCTACGAT | 410 |
| RORC | CAGCCAATCGTAGG | 411 |
| RORC | GTGAGTAGGATGAC | 412 |
| RORC | AGGTCCTCGGGGGT | 413 |
| RORC | CAGGTGACAGGCAT | 414 |
| RORC | ACAGCACCCACCAC | 415 |
| RORC | AGGAGAAACAGGAC | 416 |
| RUNX1 | AAGGGGTCCGCACG | 417 |
| RUNX1 | CGTACAGTAGCGCG | 418 |
| RUNX1 | GTAGACTTTGCAAG | 419 |
| RUNX3 | GGGGTTAGTACCCC | 420 |
| RUNX3 | GGTGCCCGCGATGG | 421 |
| RUNX3 | GCGCCCCAGCGTCA | 422 |
| RUNX3 | CGCCCCTCCCCGTT | 423 |
| RUNX3 | GAGAAGCCTGCTCG | 424 |
| RUNX3 | GGTTGGGTTGACAC | 425 |
| RUNX3 | GCGCGGGAGCTGGT | 426 |
| SATB1 | TTCTTGCGCGCCCG | 427 |
| SATB1 | AGGGCGCTGTGCGA | 428 |
| SATB1 | tggggattaaaacc | 429 |
| SATB1 | GAGGAAGGCGGCGT | 430 |
| SNAI1 | CGTCGAGCGAAGCG | 431 |
| SNAI1 | CGCGGAGGTGACAA | 432 |
| SNAI1 | ACTCCTCCGAGGCG | 433 |
| SNAI1 | GGCGCACCTGCTCG | 434 |
| sox17 | TTAGGCCCACGCCC | 435 |
| sox17 | GTAGCCTTGGGCGC | 436 |
| sox17 | GCGTCCTCTCCCAC | 437 |
| sox 17 | CCCGCAGTGTCACT | 438 |
| SOX4 | GATTATTATTGCAT | 439 |
| SOX4 | GATAAAGAGGCGCG | 440 |
| SOX4 | GGGTTCCAAGCCAA | 441 |
| SOX4 | ACACACACAGCAAA | 442 |
| SOX4 | TGGAGCATCGGTGA | 443 |
| SOX4 | GATAATTTCGTTGT | 444 |
| SOX4 | AATCGTGATGGTGT | 445 |
| SOX4 | GGACGTATTTATAC | 446 |
| SPI1 | GAGATAGTCCCCTT | 447 |
| SPI1 | AGGGATGACTTTGG | 448 |
| SUV39H1 | CGGTTGGTCCGCGC | 449 |
| SUV39H1 | CCAGCGCACGGGCA | 450 |
| SUV39H1 | GGAACCACTGCGAC | 451 |
| SUV39H1 | GCCCAGACGCACTC | 452 |
| TAL1 | CAAAGCGAGTTTCC | 453 |
| TAL1 | TAGGGAGACGAGAA | 454 |
| TAL1 | GTCCGTGTTGGGAA | 455 |
| TAL1 | CTGGAATTGGGTTC | 456 |
| TAL1 | TTACAGCGCGTCGG | 457 |
| TAL1 | GAGGCGCTTATCGG | 458 |
| TAL1 | TCGAAAGGAACCGA | 459 |
| TAL1 | GCGTACCCCCGTAG | 460 |
| TBX21 | TCTCTCCACCATGG | 461 |
| TBX21 | ctgaatttccccga | 462 |
| TBX21 | GCGCGGCAGCTCTC | 463 |
| TCF12 | CCTAGGCGGCGGAG | 464 |
| TCF12 | GGCGCGGAGGGATC | 465 |
| TCF12 | GAGGAGCCGCACCG | 466 |
| TCF12 | AGTCCTTGCATTGG | 467 |
| TCF12 | AGGTGTAGCAAGCT | 468 |
| TCF12 | CCTCCACCAATGCA | 469 |
| TCF12 | TCACAGAGCACAGA | 470 |
| TCF3 | GGGACGGTAGGGCA | 471 |
| TCF3 | CTCCCCTCCACAGA | 472 |
| TCF3 | CAGGCTGCCATCTG | 473 |
| TCF3 | GCGGGGGCTCGCCA | 474 |
| TCF3 | CCGCGAAAGTCTGA | 475 |
| TCF3 | GGAGGGATCCGCGT | 476 |
| TCF3 | GCGGGGAGAAATCA | 477 |
| TCF3 | CGAGGGTGCTCGAG | 478 |
| TCF7 | GGGACGCGCCCACT | 479 |
| TCF7 | CCTGAGAGAGACGT | 480 |
| TOX | CGCGCCGCGGCTTG | 481 |
| TOX | CGGCACACGCGCTC | 482 |
| TOX | gcggGACCAAGAGG | 483 |
| TOX | atccagaagggaca | 484 |
| TOX2 | ACAATAGCGCGCGC | 485 |
| TOX2 | GCGCGCCCCCCTCA | 486 |
| TOX2 | GAGGGGTGCCGTTC | 487 |
| TOX2 | CGCGGGCAGTGCCT | 488 |
| VEGFA | GAGCCGCGTGTGGA | 489 |
| VEGFA | CGGGCGCGTGTCTC | 490 |
| VEGFA | GCCGGGTAGCTCGG | 491 |
| VEGFA | GCGGGATCCCAAGG | 492 |
| VEGFB | CACTACCCGGAGGG | 493 |
| VEGFB | GGGGAGCGCGTGTC | 494 |
| VEGFB | GCGGCAGGGGAAAG | 495 |
| VEGFB | GGCGAGGCCGTCGG | 496 |
| VEGFC | TATGGGACCTCGGA | 497 |
| VEGFC | GGCAAGTTCAGTCC | 498 |
| VEGFC | AAACTGGGCACCGT | 499 |
| VEGFC | AGGGAGAGTGAGAG | 500 |
| VEGFD | TCCCTACCAATAGC | 501 |
| VEGFD | CTAACGTGATAACA | 502 |
| VEGFD | GATTGAGCCATTGA | 503 |
| VEGFD | TGCATGAGCATCTG | 504 |
| ZBTB16 | GCGCAGTAACCAGG | 505 |
| ZBTB16 | TCAGAATCCTTAAT | 506 |
| ZBTB16 | CGAAGCCCGGAGCG | 507 |
| ZBTB17 | GCCGAAGTACTGTA | 508 |
| ZBTB17 | agaGGGGCCACGGA | 509 |
| ZBTB17 | CCGGCTGCAACTTC | 510 |
| ZBTB17 | TTATAATTGGTGTG | 511 |
| ZBTB7B | ATTGGGCGGAGGGA | 512 |
| ZBTB7B | CCCTGTCCGCTCAT | 513 |
| ZBTB7B | CGCTCGCTGCTGGG | 514 |
| ZSCAN4 | CTGGTCACCTAGTG | 515 |
| ZSCAN4 | GCTTCCACCACACT | 516 |
| ZSCAN4 | ATCAAAAAGATGAC | 517 |
| ZSCAN4 | AGCCTGTATGAGGG | 518 |

### EMBODIMENTS

The following non-limiting embodiments provide illustrative examples of the invention, but do not limit the scope of the invention.

Embodiment 1. A method for directing a plurality of stem cells towards differentiation, the method comprising:
contacting the plurality of stem cells with a heterologous genetic circuit comprising a plurality of gate units, wherein, upon activation of the heterologous genetic circuit, the plurality of gate units operates in concert to induce a plurality of distinct modulations of a target gene in the plurality of stem cells in a sequential manner, each of the plurality of distinct modulations being necessary but individually insufficient to effect the directing of the plurality of stem cells towards differentiation, wherein the plurality of gate units comprises:
   (i) a first gate unit that is activatable, upon the activation of the heterologous genetic circuit, to induce a first distinct modulation of the plurality of distinct modulations; and
   (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell,
wherein the target gene encodes an erythroblast transformation specific (ETS) transcription factor, and
wherein, upon the contacting, the plurality of gate units operates in concert to effect the directing the plurality of stem cells towards differentiation,
optionally wherein:
(1) the plurality of stem cells comprises pluripotent stem cells; and/or
(2) upon the contacting, the plurality of gate units operates in concert to effect differentiation of the plurality of stem cells to tissue-specific progenitor cells, further optionally wherein:
   i. the tissue-specific progenitor cells comprises hematopoietic lineage cells.
      further optionally wherein:
         1. upon the contacting, the plurality of gate units operates in concert to effect the directing the plurality of stem cells to hematopoietic stem cells; and/or
         2. the hematopoietic lineage cells are characterized as being CD45+; and/or
         3. the hematopoietic lineage cells are characterized as being CD34+CD43−CD45+; and/or
   ii. the activation of the heterologous genetic circuit, at least about 10%, at least about 15%, at least about 20%, at least about 30%, or at least about 40% of the plurality of stem cells is differentiated to the tissue-specific progenitor cells; and/or
   iii. the differentiation is observed in less than about 14 days, less than about 10 days, less than about 7 days, or less than about 6 days; and/or
(3) the ETS transcription factor comprises ETS1; and/or
(4) the ETS transcription factor comprises ETV2; and/or
(5) the ETS transcription factor comprises LMO2; and/or
(6) the plurality of gate units is preconfigured such that one of the plurality of gate units is activatable, upon the activation of the heterologous genetic circuit, to modulate expression and/or activity level of an additional target gene, wherein the additional target gene comprises a T-box transcription factor (TBX) or a homeobox protein,
   further optionally wherein:
      i. the modulation of the expression and/or activity level of the additional target gene is induced prior to at least one of the plurality of distinct modulations of the target gene encoding the ETS transcription factor; and/or
      ii. the TBX comprises at least two different types of TBX; and/or
      iii. the TBX comprises TBXT and/or TBX6; and/or
      iv. the homeobox protein comprises a PRD-class homeobox protein; and/or
      v. the homeobox protein comprises MIXL1; and/or
(7) the plurality of gate units is not preconfigured to modulate expression and/or activity level of one or more members selected from the group consisting of TBXT, TBX6, and MIXL1 prior to the plurality of distinct modulations of the target gene encoding the ETS transcription factor; and/or
(8) the plurality of gate units is preconfigured such that one of the plurality of gate units is activatable, upon the activation of the heterologous genetic circuit, to modulate expression and/or activity level of a different target gene, wherein the different target gene comprises a GATA transcription factor or a basic helix-loop-helix transcription factor (bHLH); and/or
further optionally wherein:
   i. the GATA transcription factor comprises GATA2; and/or
   ii. the bHLH comprises SCL; and/or
(9) the contacting occurs in a medium that is substantially free of exogenous thrombopoietin (TPO) and/or exogenous FLT-3 Ligand (FLT3L); and/or
(10) the contacting occurs in a medium that is substantially free of exogenous interleukin (IL),
further optionally wherein:
   i. the exogenous IL comprises one or more members selected from the group consisting of IL-2, IL-3, IL-7, IL-15, and IL-21; and/or
(11) the plurality of gate unites comprises a third gate unit that is activatable upon the activation of the heterologous genetic circuit, to induce a third distinct modulation of the plurality of distinct modulations, wherein the third distinct modulation is induced subsequent to the first and second distinct modulations, such that all of the first, second, and third distinct modulations enhance or reduce the expression and/or activity level of the target gene in the plurality of stem cells; and/or
(12) the plurality of gate units is preconfigured such that expression and/or activity level of the target gene is sequentially modulated by less than or equal to three distinct gate units of the plurality of gate units; and/or
(13) less than equal to three distinct gate units of the plurality of gate units are preconfigured to sequentially modulate expression and/or activity level of the target gene; and/or
(14) the first and second distinct modulations both enhance the expression and/or activity level of the target gene in the cell; and/or
(15) the first and second distinct modulations both reduce the expression and/or activity level of the target gene in the cell; and/or
(16) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene,
   further optionally wherein:
      i. the first gene regulating moiety and the second gene regulating moiety exhibit complementarity to substantially a same polynucleotide sequence of the target gene; and/or
      ii. the first gene regulating moiety and the second gene regulating moiety exhibit complementarity to different polynucleotide sequences of the target gene; and/or
      iii. the second gate unit further comprises a second gate moiety that is activated upon the activation of the second unit, to induce activation of the second gene regulating moiety via specific binding of the second gate moiety to the second gene regulating moiety, further optionally wherein:
         1. the first gate unit further comprises a first gate moiety that is activated upon the activation of the first gate unit, to induce:
         (a) activation of the first gene regulating moiety via specific binding of the first gate moiety to the first gene regulating moiety, and
         (b) the activation of the second gate moiety via specific binding of the first gate moiety to the second gate moiety; and/or
         2. the activating moiety is capable of inducing:
         (a) activation of the first gene regulating moiety via specific binding of the activating moiety to the first gene regulating moiety, and
         (b) the activation of the second gate moiety; and/or
(17) the first gate unit, the first gate moiety, the first gene regulating moiety, the second gate, the second gate moiety, and/or the second gene regulating moiety comprises a gNA that is activatable,
   wherein, upon activation of the gNA, the gNA forms a complex with an endonuclease,
      further optionally wherein:
         i. the activatable gNA comprises a non-canonical termination sequence; and/or
         ii. the endonuclease comprises a CRISPR/Cas protein, further optionally wherein:
         1. the CRISPR/Cas protein substantially lacks cleavage activity; and/or
         2. the CRISPR/Cas protein is coupled to a transcription activator; and/or
(18) the target gene comprises a plurality of types of ETS transcription factor, wherein the first distinct modulation via the first gate unit is a modulation of a first type of ETS transcription factor, and wherein the second distinct modulation via the second gate unit is a modulation of a second type of ETS transcription factor,
   further optionally wherein (i) the first type of ETS transcription factor comprises one or more members selected from the group consisting of ETS1, ETV2, LMO2, and a combination thereof and (ii) the second type of ETS transcription factor comprises ERG.

Embodiment 2. A method for converting a plurality of stem cells (first plurality) to a plurality of hematopoietic lineage cells (second plurality), the method comprising:
culturing ex vivo the first plurality in a medium that is substantially free of one or more exogenous factors selected from the group consisting of thrombopoietin (TPO), exogenous FLT-3 Ligand (FLT3L), and interleukin (IL),
wherein, within about 14 days following the culturing, a conversion rate from the first plurality to the second plurality is at least about 3%, wherein the second plurality is CD45+,
optionally wherein the culturing comprises contacting a target gene in the first plurality by a heterologous gene regulating moiety to modulate expression and/or activity level of the target gene, wherein the target gene encodes an erythroblast transformation specific (ETS) transcription factor.

Embodiment 3. A method for converting a plurality of stem cells (first plurality) to a plurality of hematopoietic lineage cells (second plurality), the method comprising:
contacting a target gene in the first plurality by a heterologous gene regulating moiety to modulate expression and/or activity level of the target gene, wherein the target gene encodes an erythroblast transformation specific (ETS) transcription factor,
wherein the contacting effects conversion of the first plurality to the second plurality, optionally wherein:
   (1) within about 14 days following the contacting, a conversion rate from the first plurality to the second plurality is at least about 3%, wherein the second plurality is CD45+; and/or
   (2) within about 14 days following the contacting, an additional conversion rate from the first plurality to the second plurality is at least about 5%, wherein the second plurality is CD34+; and/or
   (3) the contacting occurs ex vivo in a medium that is substantially free of one or more exogenous factors selected from the group consisting of thrombopoietin (TPO), exogenous FLT-3 Ligand (FLT3L), and interleukin (IL).

Embodiment 4. The method of any one of Embodiment 1, Embodiment 2, and Embodiment 3, further optionally wherein:
(1) the medium is substantially free of the TPO; and/or
(2) the medium is substantially free of the FLT3L; and/or
(3) the medium is substantially free of the IL, further optionally wherein the IL comprises one or more members selected from the group consisting of IL-2, IL-3, IL-7, IL-15, and IL-21; and/or
(4) the second plurality is CD34+CD43−CD45+; and/or
(5) the conversion rate is observed within about 10 days, within about 7 days, or within about 6 days following the culturing or the contacting; and/or
(6) the conversion rate is at least about 10% or at least about 15%; and/or
(7) the additional conversion rate is observed within about 6 days or within about 5 days; and/or
(8) the additional conversion rate is at least about 20%, at least about 30%, at least about 35%, at least about 40%, or at least about 45%; and/or
(9) within about 7 days following the culturing, the conversion rate from the first plurality to the second plurality is at least about 3%, wherein the second plurality is CD45+ (or CD34+ and CD45+); and/or
(10) within about 7 days following the culturing, the conversion rate from the first plurality to the second plurality is at least about 6%, wherein the second plurality is CD45+ (or CD34+ and CD45+); and/or
(11) within about 7 days following the culturing, the additional conversion rate from the first plurality to the second plurality is at least about 5%, wherein the second plurality is CD34+; and/or
(12) within about 7 days following the culturing, the additional conversion rate from the first plurality to the second plurality is at least about 10%, wherein the second plurality is CD34+; and/or
(13) the ETS transcription factor comprises one or more members selected from the group consisting of ETS1, ETV2, and/or LMO2; and/or
(14) the heterologous gene regulating moiety enhances the expression and/or activity level of the target gene; and/or
(15) the heterologous gene regulating moiety reduces the expression and/or activity level of the target gene; and/or
(16) the target gene is endogenous to the first plurality; and/or
(17) the method further comprises contacting an additional target gene in the first plurality by an additional heterologous gene regulating moiety to modulate expression and/or activity level of the additional target gene, thereby to effect the conversion, wherein the additional target gene comprises one or more members selected from the group consisting of a T-box transcription factor (TBX), a homeobox protein, a GATA transcription factor, and a basic helix-loop-helix transcription factor (bHLH),
   further optionally wherein:
   a. the additional heterologous gene regulating moiety enhances the expression and/or activity level of the additional target gene; and/or
   b. the additional heterologous gene regulating moiety reduces the expression and/or activity level of the additional target gene; and/or
   c. the additional target gene is endogenous to the first plurality; and/or
(18) the heterologous gene regulating moiety comprises (i) a nucleic acid molecule and/or (ii) a polypeptide molecule exhibiting specific binding to the target gene; and/or
(19) the additional heterologous gene regulating moiety comprises (i) a nucleic acid molecule and/or (ii) a polypeptide molecule exhibiting specific binding to the additional target gene; and/or
(20) the polypeptide molecule comprises an endonuclease, and wherein the nucleic acid molecule comprises a guide nucleic acid (gNA) that forms a complex with the endonuclease,
   further optionally wherein the endonuclease comprises a CRISPR/Cas protein,
      further optionally wherein:
      i. the CRISPR/Cas protein substantially lacks cleavage activity; and/or
      ii. the CRISPR/Cas protein is coupled to a transcription activator; and/or
(21) the method further comprises directing conversion of the plurality of hematopoietic lineage cells into a lymphoid cell, further optionally wherein the lymphoid cell is selected from the group consisting of T cell, NK cell, and B cell.

Embodiment 5. A system for directing a plurality of stem cells towards differentiation, the system comprising:
a heterologous genetic circuit comprising a plurality of gate units, wherein, upon activation of the heterologous genetic circuit, the plurality of gate units operates in concert to induce a plurality of distinct modulations of a target gene in the plurality of stem cells in a sequential manner, each of the plurality of distinct modulations being necessary but individually insufficient to effect the directing of the plurality of stem cells towards differentiation, wherein the plurality of gate units comprises:
   (i) a first gate unit that is activatable, upon the activation of the heterologous genetic circuit, to induce a first distinct modulation of the plurality of distinct modulations; and
   (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell,
wherein the target gene encodes an erythroblast transformation specific (ETS) transcription factor, and
wherein, upon contacting of the plurality of stem cells by the heterologous genetic circuit, the plurality of gate units operates in concert to effect the directing the plurality of stem cells towards differentiation,
optionally wherein:
(1) the plurality of stem cells comprises pluripotent stem cells; and/or
(2) upon the contacting, the plurality of gate units operates in concert to effect differentiation of the plurality of stem cells to tissue-specific progenitor cells,
   further optionally wherein:
      i. the tissue-specific progenitor cells comprises hematopoietic lineage cells,
         further optionally wherein:
         1. upon the contacting, the plurality of gate units operates in concert to effect the directing the plurality of stem cells to hematopoietic stem cells; and/or
         2. the hematopoietic lineage cells are characterized as being CD45+; and/or
         3. the hematopoietic lineage cells are characterized as being CD34+CD43−CD45+; and/or
      ii. the activation of the heterologous genetic circuit, at least about 10%, at least about 15%, at least about 20%, at least about 30%, or at least about 40% of the plurality of stem cells is differentiated to the tissue-specific progenitor cells; and/or
      iii. the differentiation is observed in less than about 14 days, less than about 10 days, less than about 7 days, or less than about 6 days; and/or
(3) the ETS transcription factor comprises ETS1; and/or
(4) the ETS transcription factor comprises ETV2; and/or
(5) the ETS transcription factor comprises LMO2; and/or
(6) the plurality of gate units is preconfigured such that one of the plurality of gate units is activatable, upon the activation of the heterologous genetic circuit, to modulate expression and/or activity level of an additional target gene, wherein the additional target gene comprises a T-box transcription factor (TBX) or a homeobox protein,
   further optionally wherein:
      i. the modulation of the expression and/or activity level of the additional target gene is induced prior to at least one of the plurality of distinct modulations of the target gene encoding the ETS transcription factor; and/or
      ii. the TBX comprises at least two different types of TBX; and/or
      iii. the TBX comprises TBXT and/or TBX6; and/or
      iv. the homeobox protein comprises a PRD-class homeobox protein; and/or
      v. the homeobox protein comprises MIXL1; and/or
(7) the plurality of gate units is not preconfigured to modulate expression and/or activity level of one or more members selected from the group consisting of TBXT, TBX6, and MIXL1 prior to the plurality of distinct modulations of the target gene encoding the ETS transcription factor; and/or
(8) the plurality of gate units is preconfigured such that one of the plurality of gate units is activatable, upon the activation of the heterologous genetic circuit, to modulate expression and/or activity level of a different target gene, wherein the different target gene comprises a GATA transcription factor or a basic helix-loop-helix transcription factor (bHLH); and/or
further optionally wherein:
   i. the GATA transcription factor comprises GATA2; and/or
   ii. the bHLH comprises SCL; and/or
(9) the contacting occurs in a medium that is substantially free of exogenous thrombopoietin (TPO) and/or exogenous FLT-3 Ligand (FLT3L); and/or
(10) the contacting occurs in a medium that is substantially free of exogenous interleukin (IL),
further optionally wherein:
   i. the exogenous IL comprises one or more members selected from the group consisting of IL-2, IL-3, IL-7, IL-15, and IL-21; and/or
(11) the plurality of gate unites comprises a third gate unit that is activatable upon the activation of the heterologous genetic circuit, to induce a third distinct modulation of the plurality of distinct modulations, wherein the third distinct modulation is induced subsequent to the first and second distinct modulations, such that all of the first, second, and third distinct modulations enhance or reduce the expression and/or activity level of the target gene in the plurality of stem cells; and/or
(12) the plurality of gate units is preconfigured such that expression and/or activity level of the target gene is sequentially modulated by less than or equal to three distinct gate units of the plurality of gate units; and/or
(13) less than equal to three distinct gate units of the plurality of gate units are preconfigured to sequentially modulate expression and/or activity level of the target gene; and/or
(14) the first and second distinct modulations both enhance the expression and/or activity level of the target gene in the cell; and/or
(15) the first and second distinct modulations both reduce the expression and/or activity level of the target gene in the cell; and/or
(16) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene,
further optionally wherein:
   i. the first gene regulating moiety and the second gene regulating moiety exhibit complementarity to substantially a same polynucleotide sequence of the target gene; and/or
   ii. the first gene regulating moiety and the second gene regulating moiety exhibit complementarity to different polynucleotide sequences of the target gene; and/or
   iii. the second gate unit further comprises a second gate moiety that is activated upon the activation of the second unit, to induce activation of the second gene regulating moiety via specific binding of the second gate moiety to the second gene regulating moiety,
      further optionally wherein:
         1. the first gate unit further comprises a first gate moiety that is activated upon the activation of the first gate unit, to induce:
         (a) activation of the first gene regulating moiety via specific binding of the first gate moiety to the first gene regulating moiety, and
         (b) the activation of the second gate moiety via specific binding of the first gate moiety to the second gate moiety; and/or
         2. the activating moiety is capable of inducing:
         (a) activation of the first gene regulating moiety via specific binding of the activating moiety to the first gene regulating moiety, and
         (b) the activation of the second gate moiety; and/or
(17) the first gate unit, the first gate moiety, the first gene regulating moiety, the second gate, the second gate moiety, and/or the second gene regulating moiety comprises a gNA that is activatable,
   wherein, upon activation of the gNA, the gNA forms a complex with an endonuclease,
   further optionally wherein:
      i. the activatable gNA comprises a non-canonical termination sequence; and/or
      ii. the endonuclease comprises a CRISPR/Cas protein, further optionally wherein:
         1. the CRISPR/Cas protein substantially lacks cleavage activity; and/or
         2. the CRISPR/Cas protein is coupled to a transcription activator; and/or
(18) target gene comprises a plurality of types of ETS transcription factor, wherein the first distinct modulation via the first gate unit is a modulation of a first type of ETS transcription factor, and wherein the second distinct modulation via the second gate unit is a modulation of a second type of ETS transcription factor,
   further optionally wherein (i) the first type of ETS transcription factor comprises one or more members selected from the group consisting of ETS1, ETV2, LMO2, and a combination thereof and (ii) the second type of ETS transcription factor comprises ERG.

Embodiment 6. A system for converting a plurality of stem cells (first plurality) to a plurality of hematopoietic lineage cells (second plurality), the system comprising:
a medium for ex vivo culture of the first plurality, wherein the medium is substantially free of one or more exogenous factors selected from the group consisting of thrombopoietin (TPO), exogenous FLT-3 Ligand (FLT3L), and interleukin (IL),
   wherein, within about 14 days following ex vivo culturing of the first plurality in the medium, a conversion rate from the first plurality to the second plurality is at least about 3%, wherein the second plurality is CD45+,
optionally wherein the ex vivo culturing comprises contacting a target gene in the first plurality by a heterologous gene regulating moiety to modulate expression and/or activity level of the target gene, wherein the target gene encodes an erythroblast transformation specific (ETS) transcription factor.

Embodiment 7. A system for converting a plurality of stem cells (first plurality) to a plurality of hematopoietic lineage cells (second plurality), the system comprising:
a heterologous gene regulating moiety exhibiting specific binding to a target gene in the first plurality to modulate expression and/or activity level of the target gene, wherein the target gene encodes a erythroblast transformation specific (ETS) transcription factor,
wherein contacting of the target gene by the heterologous gene regulating moiety effects conversion of the first plurality to the second plurality,
optionally wherein:
(1) within about 14 days following the contacting, a conversion rate from the first plurality to the second plurality is at least about 3%, wherein the second plurality is CD45+; and/or
(2) within about 14 days following the contacting, an additional conversion rate from the first plurality to the second plurality is at least about 5%, wherein the second plurality is CD34+; and/or
(3) the contacting occurs ex vivo in a medium that is substantially free of one or more exogenous factors selected from the group consisting of thrombopoietin (TPO), exogenous FLT-3 Ligand (FLT3L), and interleukin (IL).

Embodiment 8. The system of any one of the Embodiment 5, Embodiment 6, and Embodiment 7, further optionally wherein:
(1) the medium is substantially free of the TPO; and/or
(2) the medium is substantially free of the FLT3L; and/or
(3) the medium is substantially free of the IL, further optionally wherein:
   a. the IL comprises one or more members selected from the group consisting of IL-2, IL-3, IL-7, IL-15, and IL-21; and/or
(4) the second plurality is CD34+CD43−CD45+; and/or
(5) the conversion rate is observed within about 10 days, within about 7 days, or within about 6 days following the culturing or the contacting; and/or
(6) the conversion rate is at least about 10% or at least about 15%; and/or
(7) the additional conversion rate is observed within about 6 days or within about 5 days; and/or
(8) the additional conversion rate is at least about 20%, at least about 30%, at least about 35%, at least about 40%, or at least about 45%; and/or
(9) within about 7 days following the culturing, the conversion rate from the first plurality to the second plurality is at least about 3%, wherein the second plurality is CD45+ (or CD34+ and CD45+); and/or
(10) within about 7 days following the culturing, the conversion rate from the first plurality to the second plurality is at least about 6%, wherein the second plurality is CD45+ (or CD34+ and CD45+); and/or
(11) within about 7 days following the culturing, the additional conversion rate from the first plurality to the second plurality is at least about 5%, wherein the second plurality is CD34+; and/or
(12) within about 7 days following the culturing, the additional conversion rate from the first plurality to the second plurality is at least about 10%, wherein the second plurality is CD34+; and/or
(13) the ETS transcription factor comprises one or more members selected from the group consisting of ETS1, ETV2, GATA2, SCL, and/or LMO2; and/or
(14) the ETS transcription factor comprises one or more members selected from the group consisting of ETS1, ETV2, and/or LMO2; and/or
(15) the heterologous gene regulating moiety enhances the expression and/or activity level of the target gene; and/or
(16) the heterologous gene regulating moiety reduces the expression and/or activity level of the target gene; and/or
(17) the target gene is endogenous to the first plurality; and/or
(18) the system further comprises an additional heterologous gene regulating moiety configured to bind an additional target gene in the first plurality to modulate expression and/or activity level of the additional target gene, thereby to effect the conversion, wherein the additional target gene comprises one or more members selected from the group consisting of a T-box transcription factor (TBX), a homeobox protein, a GATA transcription factor, and a basic helix-loop-helix transcription factor (bHLH),
   further optionally wherein:
      a. the additional heterologous gene regulating moiety enhances the expression and/or activity level of the additional target gene; and/or
      b. the additional heterologous gene regulating moiety reduces the expression and/or activity level of the additional target gene; and/or
      c. the additional target gene is endogenous to the first plurality; and/or
(19) the heterologous gene regulating moiety comprises (i) a nucleic acid molecule and/or (ii) a polypeptide molecule exhibiting specific binding to the target gene; and/or
(20) the additional heterologous gene regulating moiety comprises (i) a nucleic acid molecule and/or (ii) a polypeptide molecule exhibiting specific binding to the additional target gene; and/or
(21) the polypeptide molecule comprises an endonuclease, and wherein the nucleic acid molecule comprises a guide nucleic acid (gNA) that forms a complex with the endonuclease,
   further optionally wherein the endonuclease comprises a CRISPR/Cas protein,
      further optionally wherein:
      i. the CRISPR/Cas protein substantially lacks cleavage activity; and/or
      ii. the CRISPR/Cas protein is coupled to a transcription activator.

Embodiment 9. An engineered cell comprising the system of any one of Embodiment 5, Embodiment 6, Embodiment 7, and Embodiment 8.

Embodiment 10. A composition comprising the system of any one of Embodiment 5, Embodiment 6, Embodiment 7, and Embodiment 8 or the engineered cell of Embodiment 9.

Embodiment 11. A method for converting a plurality of stem cells (first plurality) to a plurality of hematopoietic lineage cells (second plurality), the method comprising:
contacting the first plurality with a heterologous genetic circuit comprising a plurality of gate units, wherein, upon activation of the heterologous genetic circuit, the plurality of gate units operates in concert to modulate expression levels of a plurality of distinct target genes in a sequential manner, wherein the plurality of gate units comprises:
   a first gate unit that is activatable, upon the activation of the heterologous genetic circuit, modulate expression of a target gene; and
   a second gate unit that is activatable, upon the activation of the first gate unit, to modulate expression of an additional target gene that is different from the target gene, such that modulation of the expression of the additional target gene is induced subsequent to modulation of the expression of the target gene,
wherein the heterologous genetic circuit is programmed such that:
   (A) the target gene comprises one or more members selected from the group consisting of TBXT, TBX6, MIXL1, and a combination thereof; and the additional target gene comprises one or more members selected from the group consisting of ETS1, ETV2, GATA2, SCL, LMO2, and a combination thereof; and/or
   (B) the target gene comprises one or more members selected from the group consisting of TBXT, TBX6, MIXL1, and a combination thereof; and the additional target gene comprises one or more members selected from the group consisting of HOXA5, HOXA9, ERG, LCOR, RUNX1, and a combination thereof; and/or
   (C) the target gene comprises one or more members selected from the group consisting of ETS1, ETV2, GATA2, SCL, LMO2, and a combination thereof; and the additional target gene comprises one or more members selected from the group consisting of HOXA5, HOXA9, ERG, LCOR, RUNX1, and a combination thereof; and/or
   (D) the plurality of gate units comprises a third gate unit that is activatable, upon the activation of the second gate unit, to modulate expression of another target gene that is different from the target gene and the additional target gene, such that modulation of the expression of the another target gene is induced subsequent to modulation of the expression of the additional target gene, wherein the target gene comprises one or more members selected from the group consisting of TBXT, TBX6, MIXL1, and a combination thereof; the additional target gene comprises one or more members selected from the group consisting of ETS1, ETV2, GATA2, SCL, LMO2, and a combination thereof; and the another target gene comprises one or more members selected from the group consisting of HOXA5, HOXA9, ERG, LCOR, RUNX1, and a combination thereof,
wherein, upon the contacting, the plurality of gate units operates in concert to effect the conversion of the first plurality to the second plurality,
optionally wherein:
(1) the heterologous genetic circuit is characterized by (A); and/or
(2) the heterologous genetic circuit is characterized by (B); and/or
(3) the heterologous genetic circuit is characterized by (C); and/or
(4) the heterologous genetic circuit is characterized by (D); and/or
(5) the plurality of gate units comprises an additional gate unit that is activatable upon the activation of the first gate unit, wherein the activation of the additional gate unit is necessary to induce activation of the second gate unit, thereby inducing a time delay between the modulation of the target gene by the first gate unit and the modulation of the additional target gene by the second gate unit; and/or
(6) the second plurality comprises hemogenic endothelium cells that are KDR− and CD34+, further optionally wherein:
   a. within about 14 days or about 7 days following the contacting, a conversion rate from the first plurality to the second plurality is at least about 5%; and/or
   b. within about 14 days or about 7 days following the contacting, a conversion rate from the first plurality to the second plurality is at least about 10%; and/or
(7) the second plurality comprises hematopoietic cells (HPCs) that are (i) CD34+ and CD43+ or (ii) CD34+ and CD45+, further optionally wherein:
   a. within about 14 days or about 7 days following the contacting, a conversion rate from the first plurality to the second plurality is at least about 3%; and/or
   b. within about 14 days or about 7 days following the contacting, a conversion rate from the first plurality to the second plurality is at least about 6%; and/or
(8) a conversion rate from the first plurality of to the second plurality via the heterologous genetic circuit is greater than that in absence of the heterologous genetic circuit by at least about 1-fold; and/or
(9) a conversion rate from the first plurality of to the second plurality via the heterologous genetic circuit is greater than that in absence of the heterologous genetic circuit by at least about 3-fold; and/or
(10) the contacting occurs ex vivo in a medium that is substantially free of one or more exogenous factors selected from the group consisting of thrombopoietin (TPO), exogenous FLT-3 Ligand (FLT3L), interleukin (IL), and a combination thereof; and/or
(11) each of (i) the modulation of the expression of the target gene, (ii) the modulation of the expression of the additional target gene, and/or (ii) the modulation of the expression of the another target gene is necessary but individually insufficient to effect the conversion of the first plurality of the second plurality; and/or
(12) a gate unit of the plurality of gate units comprises a gene regulating moiety that is activated upon activation of the gate unit, to induce modulation of a corresponding target gene via specific binding of the gene regulating moiety to the corresponding target gene; and/or
(13) the gene regulating moiety comprises a nucleic acid sequence exhibiting complementarity to at least a portion of the corresponding target gene; and/or
(14) the gene regulating moiety comprises a guide nucleic acid (gNA) molecule capable of forming a complex with an endonuclease, wherein the complex exhibits specific binding to the corresponding target gene; and/or
(15) the endonuclease comprises a CRISPR/Cas protein, further optionally wherein (i) the CRISPR/Cas protein substantially lacks cleavage activity and/or (ii) the CRISPR/Cas protein is coupled to a transcription activator; and/or
(16) the plurality of distinct target genes is endogenous to the first plurality.

Embodiment 12. A system for converting a plurality of stem cells (first plurality) to a plurality of hematopoietic lineage cells (second plurality), the system comprising:
a heterologous genetic circuit comprising a plurality of gate units, wherein, upon activation of the heterologous genetic circuit, the plurality of gate units configured to operate in concert to modulate expression levels of a plurality of distinct target genes in a sequential manner, wherein the plurality of gate units comprises:
   a first gate unit that is activatable, upon the activation of the heterologous genetic circuit, modulate expression of a target gene; and
   a second gate unit that is activatable, upon the activation of the first gate unit, to modulate expression of an additional target gene that is different from the target gene, such that modulation of the expression of the additional target gene is induced subsequent to modulation of the expression of the target gene,
wherein the heterologous genetic circuit is programmed such that:
   (A) the target gene comprises one or more members selected from the group consisting of TBXT, TBX6, MIXL1, and a combination thereof; and the additional target gene comprises one or more members selected from the group consisting of ETS1, ETV2, GATA2, SCL, LMO2, and a combination thereof; and/or
   (B) the target gene comprises one or more members selected from the group consisting of TBXT, TBX6, MIXL1, and a combination thereof; and the additional target gene comprises one or more members selected from the group consisting of HOXA5, HOXA9, ERG, LCOR, RUNX1, and a combination thereof; and/or
   (C) the target gene comprises one or more members selected from the group consisting of ETS1, ETV2, GATA2, SCL, LMO2, and a combination thereof; and the additional target gene comprises one or more members selected from the group consisting of HOXA5, HOXA9, ERG, LCOR, RUNX1, and a combination thereof; and/or
   (D) the plurality of gate units comprises a third gate unit that is activatable, upon the activation of the second gate unit, to modulate expression of another target gene that is different from the target gene and the additional target gene, such that modulation of the expression of the another target gene is induced subsequent to modulation of the expression of the additional target gene, wherein the target gene comprises one or more members selected from the group consisting of TBXT, TBX6, MIXL1, and a combination thereof; the additional target gene comprises one or more members selected from the group consisting of ETS1, ETV2, GATA2, SCL, LMO2, and a combination thereof; and the another target gene comprises one or more members selected from the group consisting of HOXA5, HOXA9, ERG, LCOR, RUNX1, and a combination thereof,
wherein, upon contacting of the first plurality by the heterologous genetic circuit, the plurality of gate units operates in concert to effect the conversion of the first plurality to the second plurality,
optionally wherein:
(1) the heterologous genetic circuit is characterized by (A); and/or
(2) the heterologous genetic circuit is characterized by (B); and/or
(3) the heterologous genetic circuit is characterized by (C); and/or
(4) the heterologous genetic circuit is characterized by (D); and/or
(5) the plurality of gate units comprises an additional gate unit that is activatable upon the activation of the first gate unit, wherein the activation of the additional gate unit is necessary to induce activation of the second gate unit, thereby inducing a time delay between the modulation of the target gene by the first gate unit and the modulation of the additional target gene by the second gate unit; and/or
(6) the second plurality comprises hemogenic endothelium cells that are KDR− and CD34+, further optionally wherein:
   a. the plurality of gate units is configured to operate in concert to effect a conversion rate from the first plurality to the second plurality of at least about 5%, within about 14 days or about 7 days following the contacting; and/or
   b. the plurality of gate units is configured to operate in concert to effect a conversion rate from the first plurality to the second plurality of at least about 10%, within about 14 days or about 7 days following the contacting; and/or
(7) the second plurality comprises hematopoietic cells (HPCs) that are (i) CD34+ and CD43+ or (ii) CD34+ and CD45+, further optionally wherein:
   a. the plurality of gate units is configured to operate in concert to effect a conversion rate from the first plurality to the second plurality of at least about 3%, within about 14 days or about 7 days following the contacting; and/or
   b. the plurality of gate units is configured to operate in concert to effect a conversion rate from the first plurality to the second plurality of at least about 6%, within about 14 days or about 7 days following the contacting; and/or
(8) a conversion rate from the first plurality of to the second plurality via the heterologous genetic circuit is greater than that in absence of the heterologous genetic circuit by at least about 1-fold; and/or
(9) a conversion rate from the first plurality of to the second plurality via the heterologous genetic circuit is greater than that in absence of the heterologous genetic circuit by at least about 3-fold; and/or
(10) the plurality of gate units is configured to operate in concert to effect the conversion ex vivo in a medium that is substantially free of one or more exogenous factors selected from the group consisting of thrombopoietin (TPO), exogenous FLT-3 Ligand (FLT3L), interleukin (IL), and a combination thereof; and/or
(11) each of (i) the modulation of the expression of the target gene, (ii) the modulation of the expression of the additional target gene, and/or (ii) the modulation of the expression of the another target gene is necessary but individually insufficient to effect the conversion of the first plurality of the second plurality; and/or
(12) a gate unit of the plurality of gate units comprises a gene regulating moiety that is activated upon activation of the gate unit, to induce modulation of a corresponding target gene via specific binding of the gene regulating moiety to the corresponding target gene; and/or
(13) the gene regulating moiety comprises a nucleic acid sequence exhibiting complementarity to at least a portion of the corresponding target gene; and/or
(14) the gene regulating moiety comprises a guide nucleic acid (gNA) molecule capable of forming a complex with an endonuclease, wherein the complex exhibits specific binding to the corresponding target gene; and/or
(15) the endonuclease comprises a CRISPR/Cas protein, further optionally wherein (i) the CRISPR/Cas protein substantially lacks cleavage activity and/or (ii) the CRISPR/Cas protein is coupled to a transcription activator; and/or
(16) the plurality of distinct target genes is endogenous to the first plurality.

Embodiment 13. An engineered cell comprising the system of Embodiment 12.

Embodiment 14. A composition comprising the system of Embodiment 12 or the engineered cell of Embodiment 13.

Systems and methods of the present disclosure may be combined with or modified by other systems and methods, such as, for example, those described in International Patent Application No. PCT/US2018/052211, International Patent Application No. PCT/US2018/052211, International Patent Application No. PCT/US2023/028169, International Patent Application No. PCT/US2023/028255, and International Patent Application No. PCT/US2023/028033, each of which is incorporated herein by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for converting a plurality of stem cells (first plurality) to a plurality of hematopoietic lineage cells (second plurality), the method comprising:
culturing ex vivo the first plurality in a medium that is substantially free of one or more exogenous factors selected from the group consisting of thrombopoietin (TPO), exogenous FLT-3 Ligand (FLT3L), and interleukin (IL),
wherein, within about 14 days following the culturing, a conversion rate from the first plurality to the second plurality is at least about 3%, wherein the second plurality is CD45+.

2. The method of claim 1, wherein the medium is substantially free of the TPO, the FLT3L, or the IL.

3.-4. (canceled)

5. The method of claim 1, wherein the IL comprises one or more members selected from the group consisting of IL-2, IL-3, IL-7, IL-15, and IL-21.

6. The method of claim 1, wherein the second plurality is CD34+CD43−CD45+.

7. The method of claim 1, wherein, within about 7 days following the culturing, the conversion rate from the first plurality to the second plurality is at least about 3%, wherein the second plurality is CD45+ (or CD34+ and CD45+).

8. The method of claim 1, wherein, within about 7 days following the culturing, the conversion rate from the first plurality to the second plurality is at least about 6%, wherein the second plurality is CD45+ (or CD34+ and CD45+).

9. (canceled)

10. The method of claim 1, wherein the culturing comprises contacting a target gene in the first plurality by a heterologous gene regulating moiety to modulate expression and/or activity level of the target gene, wherein the target gene encodes an erythroblast transformation specific (ETS) transcription factor.

11. The method of claim 10, wherein the ETS transcription factor comprises one or more members selected from the group consisting of ETS1, ETV2, GATA2, SCL, and/or LMO2.

12. (canceled)

13. The method of claim 10, wherein the heterologous gene regulating moiety enhances the expression and/or activity level of the target gene.

14. The method of claim 10, wherein the target gene is endogenous to the first plurality.

15. The method of claim 1, further comprising contacting an additional target gene in the first plurality by an additional heterologous gene regulating moiety to modulate expression and/or activity level of the additional target gene, thereby to effect the conversion, wherein the additional target gene comprises one or more members selected from the group consisting of a T-box transcription factor (TBX), a homeobox protein, a GATA transcription factor, and a basic helix-loop-helix transcription factor (bHLH).

16. The method of claim 15, wherein the additional heterologous gene regulating moiety enhances the expression and/or activity level of the additional target gene.

17. (canceled)

18. The method of claim 10, wherein each heterologous gene regulating moiety comprises (i) a nucleic acid molecule and/or (ii) a polypeptide molecule exhibiting specific binding to the target gene.

19. The method of claim 18, wherein the polypeptide molecule comprises an endonuclease, and wherein the nucleic acid molecule comprises a guide nucleic acid (gNA) that forms a complex with the endonuclease.

20. The method of claim 19, wherein the endonuclease comprises a CRISPR/Cas protein, and wherein the CRISPR/Cas protein substantially lacks cleavage activity.

21.-22. (canceled)

23. The method of claim 1, further comprising directing conversion of the plurality of hematopoietic lineage cells into a lymphoid cell.

24.-39. (canceled)

40. A method for converting a plurality of stem cells (first plurality) to a plurality of hematopoietic lineage cells (second plurality), the method comprising:
contacting a target gene in the first plurality by a heterologous gene regulating moiety to modulate expression and/or activity level of the target gene, wherein the target gene encodes an erythroblast transformation specific (ETS) transcription factor,
wherein the contacting effects conversion of the first plurality to the second plurality.

41.-79. (canceled)

80. A system for converting a plurality of stem cells (first plurality) to a plurality of hematopoietic lineage cells (second plurality), the system comprising:
a heterologous gene regulating moiety exhibiting specific binding to a target gene in the first plurality to modulate expression and/or activity level of the target gene, wherein the target gene encodes an erythroblast transformation specific (ETS) transcription factor,
wherein contacting of the target gene by the heterologous gene regulating moiety effects conversion of the first plurality to the second plurality.

81.-84. (canceled)


SEQUENCE LISTING (518 sequences)

SEQ ID NO: 1 - DNA, 14 bp, synthetic construct
  source 1..14: mol_type = other DNA
aaatgcaccgctga

SEQ ID NO: 2 - DNA, 14 bp, synthetic construct
  source 1..14: mol_type = other DNA
cgccctgctccgcg

SEQ ID NO: 3 - DNA, 14 bp, synthetic construct
  source 1..14: mol_type = other DNA
tgtagtcaggcccg

SEQ ID NO: 4 - DNA, 14 bp, synthetic construct
  source 1..14: mol_type = other DNA
cacgtgacccgctg

SEQ ID NO: 5 - DNA, 14 bp, synthetic construct
  source 1..14: mol_type = other DNA
agtgtgtcacacgg

SEQ ID NO: 6 - DNA, 14 bp, synthetic construct
  source 1..14: mol_type = other DNA
gcatggcttcacac

SEQ ID NO: 7 - DNA, 14 bp, synthetic construct
  source 1..14: mol_type = other DNA
gatgatatgcagcc

SEQ ID NO: 8 - DNA, 14 bp, synthetic construct
  source 1..14: mol_type = other DNA
cagcccaaagaaac

SEQ ID NO: 9 - DNA, 14 bp, synthetic construct
  source 1..14: mol_type = other DNA
agcgaccccgagtt

SEQ ID NO: 10 - DNA, 14 bp, synthetic construct
  source 1..14: mol_type = other DNA
gggacctcggtcgt

SEQ ID NO: 11 - DNA, 14 bp, synthetic construct
  source 1..14: mol_type = other DNA
ctcatcagcatcga

SEQ ID NO: 12 - DNA, 14 bp, synthetic construct
  source 1..14: mol_type = other DNA
agtccggccccagc

SEQ ID NO: 13 - DNA, 14 bp, synthetic construct
  source 1..14: mol_type = other DNA
gggcgatgtccgct

SEQ ID NO: 14 - DNA, 14 bp, synthetic construct
  source 1..14: mol_type = other DNA
gaaacgccctaaag

SEQ ID NO: 15 - DNA, 14 bp, synthetic construct
  source 1..14: mol_type = other DNA
cggcccaaagcgaa

SEQ ID NO: 16 - DNA, 14 bp, synthetic construct
  source 1..14: mol_type = other DNA
ggaggagcagtgcg

SEQ ID NO: 17 - DNA, 14 bp, synthetic construct
  source 1..14: mol_type = other DNA
gagcaacaccagct

SEQ ID NO: 18 - DNA, 14 bp, synthetic construct
  source 1..14: mol_type = other DNA
gctccaagagtccc

SEQ ID NO: 19 - DNA, 14 bp, synthetic construct
  source 1..14: mol_type = other DNA
gaactgcgtccccg

SEQ ID NO: 20 - DNA, 14 bp, synthetic construct
  source 1..14: mol_type = other DNA
gtacagtccgtgcg

SEQ ID NO: 21 - DNA, 14 bp, synthetic construct
  source 1..14: mol_type = other DNA
ggtcccatgcttgt

SEQ ID NO: 22 - DNA, 14 bp, synthetic construct
  source 1..14: mol_type = other DNA
ctcagtgtgatccc

SEQ ID NO: 23 - DNA, 14 bp, synthetic construct
  source 1..14: mol_type = other DNA
agacgcacatacac

SEQ ID NO: 24 - DNA, 14 bp, synthetic construct
  source 1..14: mol_type = other DNA
tccaagaatcccca

SEQ ID NO: 25 - DNA, 14 bp, synthetic construct
  source 1..14: mol_type = other DNA
ccgcgggtcagtcc

SEQ ID NO: 26 - DNA, 14 bp, synthetic construct
  source 1..14: mol_type = other DNA
gcgccccaccacta

SEQ ID NO: 27 - DNA, 14 bp, synthetic construct
  source 1..14: mol_type = other DNA
ggagggaacggtct

SEQ ID NO: 28 - DNA, 14 bp, synthetic construct
  source 1..14: mol_type = other DNA
gcccagagcatcga

SEQ ID NO: 29 - DNA, 14 bp, synthetic construct
  source 1..14: mol_type = other DNA
gcgttgcggccggt

SEQ ID NO: 30 - DNA, 14 bp, synthetic construct
  source 1..14: mol_type = other DNA
ccctgagtgcctag

SEQ ID NO: 31 - DNA, 14 bp, synthetic construct
  source 1..14: mol_type = other DNA
gtgcgctccggaga

SEQ ID NO: 32 - DNA, 14 bp, synthetic construct
  source 1..14: mol_type = other DNA
gggtgcagagcccc

SEQ ID NO: 33 - DNA, 14 bp, synthetic construct
  source 1..14: mol_type = other DNA
cttcgctaggaaag

SEQ ID NO: 34 - DNA, 14 bp, synthetic construct
  source 1..14: mol_type = other DNA
ctgtagttcccatc

SEQ ID NO: 35 - DNA, 14 bp, synthetic construct
  source 1..14: mol_type = other DNA
atgcccagtggtcg

SEQ ID NO: 36 - DNA, 14 bp, synthetic construct
  source 1..14: mol_type = other DNA
ctggctgtgcttta

SEQ ID NO: 37 - DNA, 14 bp, synthetic construct
  source 1..14: mol_type = other DNA
actcagtaacgctc

SEQ ID NO: 38 - DNA, 14 bp, synthetic construct
  source 1..14: mol_type = other DNA
ctgtgggtcggcag

SEQ ID NO: 39 - DNA, 14 bp, synthetic construct
  source 1..14: mol_type = other DNA
gggacgggctggaa

SEQ ID NO: 40 - DNA, 14 bp, synthetic construct
  source 1..14: mol_type = other DNA
caacccccgtcggg

SEQ ID NO: 41 - DNA, 14 bp, synthetic construct
  source 1..14: mol_type = other DNA
ccggggtcgaattg

SEQ ID NO: 42 - DNA, 14 bp, synthetic construct
  source 1..14: mol_type = other DNA
actaataggggagt

SEQ ID NO: 43 - DNA, 14 bp, synthetic construct
  source 1..14: mol_type = other DNA
gcggcaactggcgg

SEQ ID NO: 44 - DNA, 14 bp, synthetic construct
  source 1..14: mol_type = other DNA
gcaggatttacgac

SEQ ID NO: 45 - DNA, 14 bp, synthetic construct
  source 1..14: mol_type = other DNA
ccgcctagaatgga

SEQ ID NO: 46 - DNA, 14 bp, synthetic construct
  source 1..14: mol_type = other DNA
gccaacctccgcac

SEQ ID NO: 47 - DNA, 14 bp, synthetic construct
  source 1..14: mol_type = other DNA
ctagcaggagtcgg

SEQ ID NO: 48 - DNA, 14 bp, synthetic construct
  source 1..14: mol_type = other DNA
gcaggctgttgcgg

SEQ ID NO: 49 - DNA, 14 bp, synthetic construct
  source 1..14: mol_type = other DNA
ccgcgccttcttga

SEQ ID NO: 50 - DNA, 14 bp, synthetic construct
  source 1..14: mol_type = other DNA
ccgcacgctattaa

SEQ ID NO: 51 - DNA, 14 bp, synthetic construct
  source 1..14: mol_type = other DNA
tgccgctctacgat

SEQ ID NO: 52 - DNA, 14 bp, synthetic construct
  source 1..14: mol_type = other DNA
gcacgtgacgcgca

SEQ ID NO: 53 - DNA, 14 bp, synthetic construct
  source 1..14: mol_type = other DNA
aggttgcgactgga

SEQ ID NO: 54 - DNA, 14 bp, synthetic construct
  source 1..14: mol_type = other DNA
gagagagcgaggca

SEQ ID NO: 55 - DNA, 14 bp, synthetic construct
  source 1..14: mol_type = other DNA
caggagggctacac

SEQ ID NO: 56 - DNA, 14 bp, synthetic construct
  source 1..14: mol_type = other DNA
gcgattcccggata

SEQ ID NO: 57 - DNA, 14 bp, synthetic construct
  source 1..14: mol_type = other DNA
cgcggccttaaaaa

SEQ ID NO: 58 - DNA, 14 bp, synthetic construct
  source 1..14: mol_type = other DNA
agcggctccagaag

SEQ ID NO: 59 - DNA, 14 bp, synthetic construct
  source 1..14: mol_type = other DNA
cagcttggctcgcg

SEQ ID NO: 60 - DNA, 14 bp, synthetic construct
  source 1..14: mol_type = other DNA
ccgccgcactcccg

SEQ ID NO: 61 - DNA, 14 bp, synthetic construct
  source 1..14: mol_type = other DNA
gccgcgtagaagga

SEQ ID NO: 62 - DNA, 14 bp, synthetic construct
  source 1..14: mol_type = other DNA
gcgggactgacttc

SEQ ID NO: 63 - DNA, 14 bp, synthetic construct
  source 1..14: mol_type = other DNA
ccgtggccccggcg

SEQ ID NO: 64 - DNA, 14 bp, synthetic construct
  source 1..14: mol_type = other DNA
aggagcagtttcga

SEQ ID NO: 65 - DNA, 14 bp, synthetic construct
  source 1..14: mol_type = other DNA
gggtttatctcagg

SEQ ID NO: 66 - DNA, 14 bp, synthetic construct
  source 1..14: mol_type = other DNA
ctggacaacagtca

SEQ ID NO: 67 - DNA, 14 bp, synthetic construct
  source 1..14: mol_type = other DNA
gcagccttctgatt

SEQ ID NO: 68 - DNA, 14 bp, synthetic construct
  source 1..14: mol_type = other DNA
ccgtcaagtgtaga

SEQ ID NO: 69 - DNA, 14 bp, synthetic construct
  source 1..14: mol_type = other DNA
gaccttcccgagag

SEQ ID NO: 70 - DNA, 14 bp, synthetic construct
  source 1..14: mol_type = other DNA
gccgagggcagaga

SEQ ID NO: 71 - DNA, 14 bp, synthetic construct
  source 1..14: mol_type = other DNA
cttggcttcttaaa

SEQ ID NO: 72 - DNA, 14 bp, synthetic construct
  source 1..14: mol_type = other DNA
tcaaatacaataga

SEQ ID NO: 73 - DNA, 14 bp, synthetic construct
  source 1..14: mol_type = other DNA
caaaggtgggctcg

SEQ ID NO: 74 - DNA, 14 bp, synthetic construct
  source 1..14: mol_type = other DNA
aagcgattattccc

SEQ ID NO: 75 - DNA, 14 bp, synthetic construct
  source 1..14: mol_type = other DNA
atccacatccccga

SEQ ID NO: 76 - DNA, 14 bp, synthetic construct
  source 1..14: mol_type = other DNA
gccatgtaaggcac

SEQ ID NO: 77 - DNA, 14 bp, synthetic construct
  source 1..14: mol_type = other DNA
gcgccgaatgggag

SEQ ID NO: 78 - DNA, 14 bp, synthetic construct
  source 1..14: mol_type = other DNA
ccgcgacagtgagt

SEQ ID NO: 79 - DNA, 14 bp, synthetic construct
  source 1..14: mol_type = other DNA
accgcggaggcggc

SEQ ID NO: 80 - DNA, 14 bp, synthetic construct
  source 1..14: mol_type = other DNA
caaacaaagagctt

SEQ ID NO: 81 - DNA, 14 bp, synthetic construct
  source 1..14: mol_type = other DNA
aaggggtccgcacg

SEQ ID NO: 82 - DNA, 14 bp, synthetic construct
  source 1..14: mol_type = other DNA
cgtacagtagcgcg

SEQ ID NO: 83 - DNA, 14 bp, synthetic construct
  source 1..14: mol_type = other DNA
gtagactttgcaag

SEQ ID NO: 84 - DNA, 14 bp, synthetic construct
  source 1..14: mol_type = other DNA
gtaagcccggccgg

SEQ ID NO: 85 - DNA, 14 bp, synthetic construct
  source 1..14: mol_type = other DNA
gaaccaattgagat

SEQ ID NO: 86 - DNA, 14 bp, synthetic construct
  source 1..14: mol_type = other DNA
gccagcccgttgta

SEQ ID NO: 87 - DNA, 14 bp, synthetic construct
  source 1..14: mol_type = other DNA
ggacttaactctcc

SEQ ID NO: 88 - DNA, 14 bp, synthetic construct
  source 1..14: mol_type = other DNA
tatcagccagggcg

SEQ ID NO: 89 - DNA, 14 bp, synthetic construct
  source 1..14: mol_type = other DNA
ttcttgcgcgcccg

SEQ ID NO: 90 - DNA, 14 bp, synthetic construct
  source 1..14: mol_type = other DNA
agggcgctgtgcga

SEQ ID NO: 91 - DNA, 14 bp, synthetic construct
  source 1..14: mol_type = other DNA
tggggattaaaacc

SEQ ID NO: 92 - DNA, 14 bp, synthetic construct
  source 1..14: mol_type = other DNA
gaggaaggcggcgt

SEQ ID NO: 93 - DNA, 14 bp, synthetic construct
  source 1..14: mol_type = other DNA
gagatagtcccctt

SEQ ID NO: 94 - DNA, 14 bp, synthetic construct
  source 1..14: mol_type = other DNA
agggatgactttgg

SEQ ID NO: 95 - DNA, 14 bp, synthetic construct
  source 1..14: mol_type = other DNA
cttgcgctacatac

SEQ ID NO: 96 - DNA, 14 bp, synthetic construct
  source 1..14: mol_type = other DNA
ctctgccacaccac

SEQ ID NO: 97 - DNA, 14 bp, synthetic construct
  source 1..14: mol_type = other DNA
cgaggtcacggcgt

SEQ ID NO: 98 - DNA, 14 bp, synthetic construct
  source 1..14: mol_type = other DNA
ccgcgtggtccgtc

SEQ ID NO: 99 - DNA, 14 bp, synthetic construct
  source 1..14: mol_type = other DNA
ggggcagcgtccca

SEQ ID NO: 100 - DNA, 14 bp, synthetic construct
  source 1..14: mol_type = other DNA
ccagaccttctggc

SEQ ID NO: 101 - DNA, 14 bp, synthetic construct
  source 1..14: mol_type = other DNA
gctttcacggaagg

SEQ ID NO: 102 - DNA, 14 bp, synthetic construct
  source 1..14: mol_type = other DNA
ctgcgcatcgggtg

SEQ ID NO: 103 - DNA, 14 bp, synthetic construct
  source 1..14: mol_type = other DNA
ccgcacttccgcga

SEQ ID NO: 104 - DNA, 14 bp, synthetic construct
  source 1..14: mol_type = other DNA
gcgccaagcggtgt

SEQ ID NO: 105 - DNA, 14 bp, synthetic construct
  source 1..14: mol_type = other DNA
caaagcgagtttcc

SEQ ID NO: 106 - DNA, 14 bp, synthetic construct
  source 1..14: mol_type = other DNA
tagggagacgagaa

SEQ ID NO: 107 - DNA, 14 bp, synthetic construct
  source 1..14: mol_type = other DNA
gtccgtgttgggaa

SEQ ID NO: 108 - DNA, 14 bp, synthetic construct
  source 1..14: mol_type = other DNA
ctggaattgggttc

SEQ ID NO: 109 - DNA, 14 bp, synthetic construct
  source 1..14: mol_type = other DNA
ttacagcgcgtcgg

SEQ ID NO: 110 - DNA, 14 bp, synthetic construct
  source 1..14: mol_type = other DNA
gaggcgcttatcgg

SEQ ID NO: 111 - DNA, 14 bp, synthetic construct
  source 1..14: mol_type = other DNA
tcgaaaggaaccga

SEQ ID NO: 112 - DNA, 14 bp, synthetic construct
  source 1..14: mol_type = other DNA
gcgtacccccgtag

SEQ ID NO: 113-116 - skipped (4 entries)

SEQ ID NO: 117 - DNA, 23 bp, synthetic construct
  source 1..23: mol_type = other DNA
cctacactcgacatcggtagcta

SEQ ID NO: 118 - DNA, 23 bp, synthetic construct
  source 1..23: mol_type = other DNA
ccctggcggaacgttcgagtaat

SEQ ID NO: 119 - DNA, 23 bp, synthetic construct
  source 1..23: mol_type = other DNA
cctacacgatagtggtcgtgcgc

SEQ ID NO: 120 - DNA, 23 bp, synthetic construct
  source 1..23: mol_type = other DNA
cccgaactctctatcgaacgagt

SEQ ID NO: 121 - DNA, 23 bp, synthetic construct
  source 1..23: mol_type = other DNA
ccatggcacgatcgacacggagg

SEQ ID NO: 122 - DNA, 23 bp, synthetic construct
  source 1..23: mol_type = other DNA
ccaaagctcattcgcgactgagc

SEQ ID NO: 123 - DNA, 23 bp, synthetic construct
  source 1..23: mol_type = other DNA
ccgacgtctacggacgggagcta

SEQ ID NO: 124 - DNA, 23 bp, synthetic construct
  source 1..23: mol_type = other DNA
cccgagaggtagtagacgacgca

SEQ ID NO: 125 - skipped

SEQ ID NO: 126 - DNA, 23 bp, synthetic construct
  source 1..23: mol_type = other DNA
cccgtcgtcacgtatgcgtagca

SEQ ID NO: 127 - DNA, 23 bp, synthetic construct
  source 1..23: mol_type = other DNA
cccgtcgtcacgtatgcgtagca

SEQ ID NO: 128 - DNA, 23 bp, synthetic construct
  source 1..23: mol_type = other DNA
cccgagaggtagtagacgacgca

SEQ ID NO: 129 - DNA, 23 bp, synthetic construct
  source 1..23: mol_type = other DNA
ccgacgtctacggacgggagcta

SEQ ID NO: 130 - DNA, 23 bp, synthetic construct
  source 1..23: mol_type = other DNA
ccaaagctcattcgcgactgagc

SEQ ID NO: 131 - DNA, 23 bp, synthetic construct
  source 1..23: mol_type = other DNA
ccatggcacgatcgacacggagg

SEQ ID NO: 132 - DNA, 23 bp, synthetic construct
  source 1..23: mol_type = other DNA
cccgaactctctatcgaacgagt

SEQ ID NO: 133 - DNA, 23 bp, synthetic construct
  source 1..23: mol_type = other DNA
cctacacgatagtggtcgtgcgc

SEQ ID NO: 134 - DNA, 23 bp, synthetic construct
  source 1..23: mol_type = other DNA
ccctggcggaacgttcgagtaat

SEQ ID NO: 135 - DNA, 23 bp, synthetic construct
  source 1..23: mol_type = other DNA
cctacactcgacatcggtagcta

SEQ ID NO: 136 - DNA, 29 bp, synthetic construct
  source 1..29: mol_type = other DNA
ttttttttcagccaactccaatttttttt

SEQ ID NO: 137-144 - skipped (8 entries)

SEQ ID NO: 145 - DNA, 29 bp, synthetic construct
  source 1..29: mol_type = other DNA
ttttttttcagccaactccaatttttttt

SEQ ID NO: 146-153 - skipped (8 entries)

SEQ ID NO: 154 - DNA, 23 bp, synthetic construct
  source 1..23: mol_type = other DNA
tagctaccgatgtcgagtgtagg

SEQ ID NO: 155 - DNA, 23 bp, synthetic construct
  source 1..23: mol_type = other DNA
attactcgaacgttccgccaggg

SEQ ID NO: 156 - DNA, 23 bp, synthetic construct
  source 1..23: mol_type = other DNA
gcgcacgaccactatcgtgtagg

SEQ ID NO: 157 - DNA, 23 bp, synthetic construct
  source 1..23: mol_type = other DNA
actcgttcgatagagagttcggg

SEQ ID NO: 158 - DNA, 23 bp, synthetic construct
  source 1..23: mol_type = other DNA
cctccgtgtcgatcgtgccatgg

SEQ ID NO: 159 - DNA, 23 bp, synthetic construct
  source 1..23: mol_type = other DNA
gctcagtcgcgaatgagctttgg

SEQ ID NO: 160 - DNA, 23 bp, synthetic construct
  source 1..23: mol_type = other DNA
tagctcccgtccgtagacgtcgg

SEQ ID NO: 161 - DNA, 23 bp, synthetic construct
  source 1..23: mol_type = other DNA
tgcgtcgtctactacctctcggg

SEQ ID NO: 162 - DNA, 23 bp, synthetic construct
  source 1..23: mol_type = other DNA
tgctacgcatacgtgacgacggg

SEQ ID NO: 163 - DNA, 23 bp, synthetic construct
  source 1..23: mol_type = other DNA
tgctacgcatacgtgacgacggg

SEQ ID NO: 164 - DNA, 23 bp, synthetic construct
  source 1..23: mol_type = other DNA
tgcgtcgtctactacctctcggg

SEQ ID NO: 165 - DNA, 23 bp, synthetic construct
  source 1..23: mol_type = other DNA
tagctcccgtccgtagacgtcgg

SEQ ID NO: 166 - DNA, 23 bp, synthetic construct
  source 1..23: mol_type = other DNA
gctcagtcgcgaatgagctttgg

SEQ ID NO: 167 - DNA, 23 bp, synthetic construct
  source 1..23: mol_type = other DNA
cctccgtgtcgatcgtgccatgg

SEQ ID NO: 168 - DNA, 23 bp, synthetic construct
  source 1..23: mol_type = other DNA
actcgttcgatagagagttcggg

SEQ ID NO: 169 - DNA, 23 bp, synthetic construct
  source 1..23: mol_type = other DNA
gcgcacgaccactatcgtgtagg

SEQ ID NO: 170 - DNA, 23 bp, synthetic construct
  source 1..23: mol_type = other DNA
attactcgaacgttccgccaggg

SEQ ID NO: 171 - DNA, 23 bp, synthetic construct
  source 1..23: mol_type = other DNA
tagctaccgatgtcgagtgtagg

SEQ ID NO: 172 - DNA, 20 bp, synthetic construct
  source 1..20: mol_type = other DNA
tagctaccgatgtcgagtgt

SEQ ID NO: 173 - DNA, 20 bp, synthetic construct
  source 1..20: mol_type = other DNA
attactcgaacgttccgcca

SEQ ID NO: 174 - DNA, 20 bp, synthetic construct
  source 1..20: mol_type = other DNA
gcgcacgaccactatcgtgt

SEQ ID NO: 175 - DNA, 20 bp, synthetic construct
  source 1..20: mol_type = other DNA
actcgttcgatagagagttc

SEQ ID NO: 176 - DNA, 20 bp, synthetic construct
  source 1..20: mol_type = other DNA
cctccgtgtcgatcgtgcca

SEQ ID NO: 177 - DNA, 20 bp, synthetic construct
  source 1..20: mol_type = other DNA
gctcagtcgcgaatgagctt

SEQ ID NO: 178 - DNA, 20 bp, synthetic construct
  source 1..20: mol_type = other DNA
tagctcccgtccgtagacgt

SEQ ID NO: 179 - DNA, 20 bp, synthetic construct
  source 1..20: mol_type = other DNA
tgcgtcgtctactacctctc

SEQ ID NO: 180 - DNA, 20 bp, synthetic construct
  source 1..20: mol_type = other DNA
tgctacgcatacgtgacgac

SEQ ID NO: 181 - DNA, 20 bp, synthetic construct
  source 1..20: mol_type = other DNA
tgctacgcatacgtgacgac

SEQ ID NO: 182 - DNA, 20 bp, synthetic construct
  source 1..20: mol_type = other DNA
tgcgtcgtctactacctctc

SEQ ID NO: 183 - DNA, 20 bp, synthetic construct
  source 1..20: mol_type = other DNA
tagctcccgtccgtagacgt

SEQ ID NO: 184 - DNA, 20 bp, synthetic construct
  source 1..20: mol_type = other DNA
gctcagtcgcgaatgagctt

SEQ ID NO: 185 - DNA, 20 bp, synthetic construct
  source 1..20: mol_type = other DNA
cctccgtgtcgatcgtgcca

SEQ ID NO: 186 - DNA, 20 bp, synthetic construct
  source 1..20: mol_type = other DNA
actcgttcgatagagagttc

SEQ ID NO: 187 - DNA, 20 bp, synthetic construct
  source 1..20: mol_type = other DNA
gcgcacgaccactatcgtgt

SEQ ID NO: 188 - DNA, 20 bp, synthetic construct
  source 1..20: mol_type = other DNA
attactcgaacgttccgcca

SEQ ID NO: 189 - DNA, 20 bp, synthetic construct
  source 1..20: mol_type = other DNA
tagctaccgatgtcgagtgt

SEQ ID NO: 190 - DNA, 14 bp, synthetic construct
  source 1..14: mol_type = other DNA
gcgtgtggacgcca

SEQ ID NO: 191 - DNA, 14 bp, synthetic construct
  source 1..14: mol_type = other DNA
tgcaagttcaagtg

SEQ ID NO: 192 - DNA, 14 bp, synthetic construct
  source 1..14: mol_type = other DNA
gcccgcaccgaccc

SEQ ID NO: 193 - DNA, 14 bp, synthetic construct
  source 1..14: mol_type = other DNA
agtcggagcactgg

SEQ ID NO: 194 - DNA, 14 bp, synthetic construct
  source 1..14: mol_type = other DNA
tctcggtctctcta

SEQ ID NO: 195 - DNA, 14 bp, synthetic construct
  source 1..14: mol_type = other DNA
cacaggaaaccggt

SEQ ID NO: 196 - DNA, 14 bp, synthetic construct
  source 1..14: mol_type = other DNA
gcgcgtcccgccgg

SEQ ID NO: 197 - DNA, 14 bp, synthetic construct
  source 1..14: mol_type = other DNA
gcgtgcggttcccc

SEQ ID NO: 198 - DNA, 14 bp, synthetic construct
  source 1..14: mol_type = other DNA
gggtggctcagagg

SEQ ID NO: 199 - DNA, 14 bp, synthetic construct
  source 1..14: mol_type = other DNA
cgggagagtactcc

SEQ ID NO: 200 - DNA, 14 bp, synthetic construct
  source 1..14: mol_type = other DNA
gccgccctcgatcc

SEQ ID NO: 201 - DNA, 14 bp, synthetic construct
  source 1..14: mol_type = other DNA
ctccacctcaccct

SEQ ID NO: 202 - DNA, 14 bp, synthetic construct
  source 1..14: mol_type = other DNA
gccggtaactcagc

SEQ ID NO: 203 - DNA, 14 bp, synthetic construct
  source 1..14: mol_type = other DNA
cggacggatgaaat

SEQ ID NO: 204 - DNA, 14 bp, synthetic construct
  source 1..14: mol_type = other DNA
tcgcggtagatctg

SEQ ID NO: 205 - DNA, 14 bp, synthetic construct
  source 1..14: mol_type = other DNA
gcccgagacgcaaa

SEQ ID NO: 206 - DNA, 14 bp, synthetic construct
  source 1..14: mol_type = other DNA
tagctcaacgagag

SEQ ID NO: 207 - DNA, 14 bp, synthetic construct
  source 1..14: mol_type = other DNA
aaatgcaccgctga

SEQ ID NO: 208 - DNA, 14 bp, synthetic construct
  source 1..14: mol_type = other DNA
cgccctgctccgcg

SEQ ID NO: 209 - DNA, 14 bp, synthetic construct
  source 1..14: mol_type = other DNA
tgtagtcaggcccg

SEQ ID NO: 210 - DNA, 14 bp, synthetic construct
  source 1..14: mol_type = other DNA
cacgtgacccgctg

SEQ ID NO: 211 - DNA, 14 bp, synthetic construct
  source 1..14: mol_type = other DNA
gcatccttcccgcg

SEQ ID NO: 212 - DNA, 14 bp, synthetic construct
  source 1..14: mol_type = other DNA
ggtcccaaaggcgg

SEQ ID NO: 213 - DNA, 14 bp, synthetic construct
  source 1..14: mol_type = other DNA
aagactaaaaaggg

SEQ ID NO: 214 - DNA, 14 bp, synthetic construct
  source 1..14: mol_type = other DNA
atatggaaggtcac

SEQ ID NO: 215 - DNA, 14 bp, synthetic construct
  source 1..14: mol_type = other DNA
ccgactggctgacg

SEQ ID NO: 216 - DNA, 14 bp, synthetic construct
  source 1..14: mol_type = other DNA
gatggcggggttaa

SEQ ID NO: 217 - DNA, 14 bp, synthetic construct
  source 1..14: mol_type = other DNA
agctagaggccggg

SEQ ID NO: 218 - DNA, 14 bp, synthetic construct
  source 1..14: mol_type = other DNA
gctgggggacctag

SEQ ID NO: 219 - DNA, 14 bp, synthetic construct
  source 1..14: mol_type = other DNA
ggagacacacacac

SEQ ID NO: 220 - DNA, 14 bp, synthetic construct
  source 1..14: mol_type = other DNA
gcttctgatccccc

SEQ ID NO: 221 - DNA, 14 bp, synthetic construct
  source 1..14: mol_type = other DNA
tgttcccccgccat

SEQ ID NO: 222 - DNA, 14 bp, synthetic construct
  source 1..14: mol_type = other DNA
aggcaggtcctaaa

SEQ ID NO: 223 - DNA, 14 bp, synthetic construct
  source 1..14: mol_type = other DNA
accaggcatctcaa

SEQ ID NO: 224 - DNA, 14 bp, synthetic construct
  source 1..14: mol_type = other DNA
taagaatgcatcct

SEQ ID NO: 225 - DNA, 14 bp, synthetic construct
  source 1..14: mol_type = other DNA
ccgcgagatcccgg

SEQ ID NO: 226 - DNA, 14 bp, synthetic construct
  source 1..14: mol_type = other DNA
tgtttaggagtcac

SEQ ID NO: 227 - DNA, 14 bp, synthetic construct
  source 1..14: mol_type = other DNA
cccgagaccccaag

SEQ ID NO: 228 - DNA, 14 bp, synthetic construct
  source 1..14: mol_type = other DNA
tcaactcgcgctgc

SEQ ID NO: 229 - DNA, 14 bp, synthetic construct
  source 1..14: mol_type = other DNA
tcatctacacgcaa

SEQ ID NO: 230 - DNA, 14 bp, synthetic construct
  source 1..14: mol_type = other DNA
tcccgggccgcgat

SEQ ID NO: 231 - DNA, 14 bp, synthetic construct
  source 1..14: mol_type = other DNA
ggagaccctcctat

SEQ ID NO: 232 - DNA, 14 bp, synthetic construct
  source 1..14: mol_type = other DNA
aggcgcgggctagt

SEQ ID NO: 233 - DNA, 14 bp, synthetic construct
  source 1..14: mol_type = other DNA
gagcaagaggtacg

SEQ ID NO: 234 - DNA, 14 bp, synthetic construct
  source 1..14: mol_type = other DNA
agtgtgtcacacgg

SEQ ID NO: 235 - DNA, 14 bp, synthetic construct
  source 1..14: mol_type = other DNA
gcatggcttcacac

SEQ ID NO: 236 - DNA, 14 bp, synthetic construct
  source 1..14: mol_type = other DNA
gatgatatgcagcc

SEQ ID NO: 237 - DNA, 14 bp, synthetic construct
  source 1..14: mol_type = other DNA
cagcccaaagaaac

SEQ ID NO: 238 - DNA, 14 bp, synthetic construct
  source 1..14: mol_type = other DNA
agcgaccccgagtt

SEQ ID NO: 239 - DNA, 14 bp, synthetic construct
  source 1..14: mol_type = other DNA
gggacctcggtcgt

SEQ ID NO: 240 - DNA, 14 bp, synthetic construct
  source 1..14: mol_type = other DNA
ctcatcagcatcga

SEQ ID NO: 241 - DNA, 14 bp, synthetic construct
  source 1..14: mol_type = other DNA
agtccggccccagc

SEQ ID NO: 242 - DNA, 14 bp, synthetic construct
  source 1..14: mol_type = other DNA
gggcgatgtccgct

SEQ ID NO: 243 - DNA, 14 bp, synthetic construct
  source 1..14: mol_type = other DNA
gaaacgccctaaag

SEQ ID NO: 244 - DNA, 14 bp, synthetic construct
  source 1..14: mol_type = other DNA
cggcccaaagcgaa

SEQ ID NO: 245 - DNA, 14 bp, synthetic construct
  source 1..14: mol_type = other DNA
ggaggagcagtgcg

SEQ ID NO: 246 - DNA, 14 bp, synthetic construct
  source 1..14: mol_type = other DNA
gagcaacaccagct

SEQ ID NO: 247 - DNA, 14 bp, synthetic construct
  source 1..14: mol_type = other DNA
gctccaagagtccc

SEQ ID NO: 248 - DNA, 14 bp, synthetic construct
  source 1..14: mol_type = other DNA
gaactgcgtccccg

SEQ ID NO: 249 - DNA, 14 bp, synthetic construct
  source 1..14: mol_type = other DNA
atgcgtcctagcag

SEQ ID NO: 250 - DNA, 14 bp, synthetic construct
  source 1..14: mol_type = other DNA
gtagtgcgtccgcg

SEQ ID NO: 251 - DNA, 14 bp, synthetic construct
  source 1..14: mol_type = other DNA
ctggagaactggta

SEQ ID NO: 252 - DNA, 14 bp, synthetic construct
  source 1..14: mol_type = other DNA
ggcgctggcagggg

SEQ ID NO: 253 - DNA, 14 bp, synthetic construct
  source 1..14: mol_type = other DNA
ctggtgacgtcaag

SEQ ID NO: 254 - DNA, 14 bp, synthetic construct
  source 1..14: mol_type = other DNA
cccgaaactgggga

SEQ ID NO: 255 - DNA, 14 bp, synthetic construct
  source 1..14: mol_type = other DNA
agggaagcgagcct

SEQ ID NO: 256 - DNA, 14 bp, synthetic construct
  source 1..14: mol_type = other DNA
gggcgggtggcccg

SEQ ID NO: 257 - DNA, 14 bp, synthetic construct
  source 1..14: mol_type = other DNA
gggctcaaccacgg

SEQ ID NO: 258 - DNA, 14 bp, synthetic construct
  source 1..14: mol_type = other DNA
atgctcacgagatt

SEQ ID NO: 259 - DNA, 14 bp, synthetic construct
  source 1..14: mol_type = other DNA
gcagccaacaccga

SEQ ID NO: 260 - DNA, 14 bp, synthetic construct
  source 1..14: mol_type = other DNA
acagggaaaggccg

SEQ ID NO: 261 - DNA, 14 bp, synthetic construct
  source 1..14: mol_type = other DNA
ggccaatgggcatg

SEQ ID NO: 262 - DNA, 14 bp, synthetic construct
  source 1..14: mol_type = other DNA
tcccgtaagtcggg

SEQ ID NO: 263 - DNA, 14 bp, synthetic construct
  source 1..14: mol_type = other DNA
cagagccgagtact

SEQ ID NO: 264 - DNA, 14 bp, synthetic construct
  source 1..14: mol_type = other DNA
tccccgtggaaaac

SEQ ID NO: 265 - DNA, 14 bp, synthetic construct
  source 1..14: mol_type = other DNA
cacaccgtacagcg

SEQ ID NO: 266 - DNA, 14 bp, synthetic construct
  source 1..14: mol_type = other DNA
acagggccaacccg

SEQ ID NO: 267 - DNA, 14 bp, synthetic construct
  source 1..14: mol_type = other DNA
ctctatgtgtggat

SEQ ID NO: 268 - DNA, 14 bp, synthetic construct
  source 1..14: mol_type = other DNA
ggtcccatgcttgt

SEQ ID NO: 269 - DNA, 14 bp, synthetic construct
  source 1..14: mol_type = other DNA
ctcagtgtgatccc

SEQ ID NO: 270 - DNA, 14 bp, synthetic construct
  source 1..14: mol_type = other DNA
agacgcacatacac

SEQ ID NO: 271 - DNA, 14 bp, synthetic construct
  source 1..14: mol_type = other DNA
tccaagaatcccca

SEQ ID NO: 272 - DNA, 14 bp, synthetic construct
  source 1..14: mol_type = other DNA
ccgcgggtcagtcc

SEQ ID NO: 273 - DNA, 14 bp, synthetic construct
  source 1..14: mol_type = other DNA
gcgccccaccacta

SEQ ID NO: 274 - DNA, 14 bp, synthetic construct
  source 1..14: mol_type = other DNA
ggagggaacggtct

SEQ ID NO: 275 - DNA, 14 bp, synthetic construct
  source 1..14: mol_type = other DNA
gcccagagcatcga

SEQ ID NO: 276 - DNA, 14 bp, synthetic construct
  source 1..14: mol_type = other DNA
gcgttgcggccggt

SEQ ID NO: 277 - DNA, 14 bp, synthetic construct
  source 1..14: mol_type = other DNA
ccctgagtgcctag

SEQ ID NO: 278 - DNA, 14 bp, synthetic construct
  source 1..14: mol_type = other DNA
gtgcgctccggaga

SEQ ID NO: 279 - DNA, 14 bp, synthetic construct
  source 1..14: mol_type = other DNA
gcggccacatttaa

SEQ ID NO: 280 - DNA, 14 bp, synthetic construct
  source 1..14: mol_type = other DNA
cccaaacccgctcc

SEQ ID NO: 281 - DNA, 14 bp, synthetic construct
  source 1..14: mol_type = other DNA
ttccggtcagtgga

SEQ ID NO: 282 - DNA, 14 bp, synthetic construct
  source 1..14: mol_type = other DNA
cctcccccccggcg

SEQ ID NO: 283 - DNA, 14 bp, synthetic construct
  source 1..14: mol_type = other DNA
cttcgctaggaaag

SEQ ID NO: 284 - DNA, 14 bp, synthetic construct
  source 1..14: mol_type = other DNA
ctgtagttcccatc

SEQ ID NO: 285 - DNA, 14 bp, synthetic construct
  source 1..14: mol_type = other DNA
atgcccagtggtcg

SEQ ID NO: 286 - DNA, 14 bp, synthetic construct
  source 1..14: mol_type = other DNA
ctggctgtgcttta

SEQ ID NO: 287 - DNA, 14 bp, synthetic construct
  source 1..14: mol_type = other DNA
actcagtaacgctc

SEQ ID NO: 288 - DNA, 14 bp, synthetic construct
  source 1..14: mol_type = other DNA
ctgtgggtcggcag

SEQ ID NO: 289 - DNA, 14 bp, synthetic construct
  source 1..14: mol_type = other DNA
gggacgggctggaa

SEQ ID NO: 290 - DNA, 14 bp, synthetic construct
  source 1..14: mol_type = other DNA
cgctccaagtgtcg

SEQ ID NO: 291 - DNA, 14 bp, synthetic construct
  source 1..14: mol_type = other DNA
ctcgacacttggag

SEQ ID NO: 292 - DNA, 14 bp, synthetic construct
  source 1..14: mol_type = other DNA
aatgaagagagcgc

SEQ ID NO: 293 - DNA, 14 bp, synthetic construct
  source 1..14: mol_type = other DNA
tgaaaatcagggag

SEQ ID NO: 294 - DNA, 14 bp, synthetic construct
  source 1..14: mol_type = other DNA
cctctatatatatc

SEQ ID NO: 295 - DNA, 14 bp, synthetic construct
  source 1..14: mol_type = other DNA
ccaggcacaaggtc

SEQ ID NO: 296 - DNA, 14 bp, synthetic construct
  source 1..14: mol_type = other DNA
ctgaaagttactgt

SEQ ID NO: 297 - DNA, 14 bp, synthetic construct
  source 1..14: mol_type = other DNA
caacgtcaatcaaa

SEQ ID NO: 298 - DNA, 14 bp, synthetic construct
  source 1..14: mol_type = other DNA
gcgcgtcccggggg

SEQ ID NO: 299 - DNA, 14 bp, synthetic construct
  source 1..14: mol_type = other DNA
gcggagcctatcgc

SEQ ID NO: 300 - DNA, 14 bp, synthetic construct
  source 1..14: mol_type = other DNA
ctggtgccccgccg

SEQ ID NO: 301 - DNA, 14 bp, synthetic construct
  source 1..14: mol_type = other DNA
cggccaatggcgcg

SEQ ID NO: 302 - DNA, 14 bp, synthetic construct
  source 1..14: mol_type = other DNA
ccaacgtggctggt

SEQ ID NO: 303 - DNA, 14 bp, synthetic construct
  source 1..14: mol_type = other DNA
atttatatcaatcg

SEQ ID NO: 304 - DNA, 14 bp, synthetic construct
  source 1..14: mol_type = other DNA
caagggcccttcta

SEQ ID NO: 305 - DNA, 14 bp, synthetic construct
  source 1..14: mol_type = other DNA
ccggggtcgaattg

SEQ ID NO: 306 - DNA, 14 bp, synthetic construct
  source 1..14: mol_type = other DNA
actaataggggagt

SEQ ID NO: 307 - DNA, 14 bp, synthetic construct
  source 1..14: mol_type = other DNA
gcggcaactggcgg

SEQ ID NO: 308 - DNA, 14 bp, synthetic construct
  source 1..14: mol_type = other DNA
ccgcctagaatgga

SEQ ID NO: 309 - DNA, 14 bp, synthetic construct
  source 1..14: mol_type = other DNA
gccaacctccgcac

SEQ ID NO: 310 - DNA, 14 bp, synthetic construct
  source 1..14: mol_type = other DNA
ctagcaggagtcgg

SEQ ID NO: 311 - DNA, 14 bp, synthetic construct
  source 1..14: mol_type = other DNA
gcaggctgttgcgg

SEQ ID NO: 312 - DNA, 14 bp, synthetic construct
  source 1..14: mol_type = other DNA
ccgcgccttcttga

SEQ ID NO: 313 - DNA, 14 bp, synthetic construct
  source 1..14: mol_type = other DNA
aggttgcgactgga

SEQ ID NO: 314 - DNA, 14 bp, synthetic construct
  source 1..14: mol_type = other DNA
gagagagcgaggca

SEQ ID NO: 315 - DNA, 14 bp, synthetic construct
  source 1..14: mol_type = other DNA
caggagggctacac

SEQ ID NO: 316 - DNA, 14 bp, synthetic construct
  source 1..14: mol_type = other DNA
ggcgcatcacgcgg

SEQ ID NO: 317 - DNA, 14 bp, synthetic construct
  source 1..14: mol_type = other DNA
gagtgatccctgac

SEQ ID NO: 318 - DNA, 14 bp, synthetic construct
  source 1..14: mol_type = other DNA
cggagcagactctc

SEQ ID NO: 319 - DNA, 14 bp, synthetic construct
  source 1..14: mol_type = other DNA
acatctggcgcaat

SEQ ID NO: 320 - DNA, 14 bp, synthetic construct
  source 1..14: mol_type = other DNA
agccaatccccgcg

SEQ ID NO: 321 - DNA, 14 bp, synthetic construct
  source 1..14: mol_type = other DNA
agcccgagcccggc

SEQ ID NO: 322 - DNA, 14 bp, synthetic construct
  source 1..14: mol_type = other DNA
gtacagtaagtgcg

SEQ ID NO: 323 - DNA, 14 bp, synthetic construct
  source 1..14: mol_type = other DNA
gcgcacgctcgccg

SEQ ID NO: 324 - DNA, 14 bp, synthetic construct
  source 1..14: mol_type = other DNA
cagtgttgggctaa

SEQ ID NO: 325 - DNA, 14 bp, synthetic construct
  source 1..14: mol_type = other DNA
ggaagcgctgatac

SEQ ID NO: 326 - DNA, 14 bp, synthetic construct
  source 1..14: mol_type = other DNA
cgcgcgtaacccgt

SEQ ID NO: 327 - DNA, 14 bp, synthetic construct
  source 1..14: mol_type = other DNA
cgctcccggccgac

SEQ ID NO: 328 - DNA, 14 bp, synthetic construct
  source 1..14: mol_type = other DNA
acgcaagggcgcgg

SEQ ID NO: 329 - DNA, 14 bp, synthetic construct
  source 1..14: mol_type = other DNA
cgccgcatcccgtg

SEQ ID NO: 330 - DNA, 14 bp, synthetic construct
  source 1..14: mol_type = other DNA
gggctccaccggat

SEQ ID NO: 331 - DNA, 14 bp, synthetic construct
  source 1..14: mol_type = other DNA
gagtgcgcgcgtcg

SEQ ID NO: 332 - DNA, 14 bp, synthetic construct
  source 1..14: mol_type = other DNA
cgcgggcggagggc

SEQ ID NO: 333 - DNA, 14 bp, synthetic construct
  source 1..14: mol_type = other DNA
aagtgctgccccgg

SEQ ID NO: 334 - DNA, 14 bp, synthetic construct
  source 1..14: mol_type = other DNA
caccacgtactttc

SEQ ID NO: 335 - DNA, 14 bp, synthetic construct
  source 1..14: mol_type = other DNA
gattcttaaggaac

SEQ ID NO: 336 - DNA, 14 bp, synthetic construct
  source 1..14: mol_type = other DNA
ggcagaacccagct

SEQ ID NO: 337 - DNA, 14 bp, synthetic construct
  source 1..14: mol_type = other DNA
ctatcttaggctag

SEQ ID NO: 338 - DNA, 14 bp, synthetic construct
  source 1..14: mol_type = other DNA
ctaaccacagacaa

SEQ ID NO: 339 - DNA, 14 bp, synthetic construct
  source 1..14: mol_type = other DNA
agggaatatccagg

SEQ ID NO: 340 - DNA, 14 bp, synthetic construct
  source 1..14: mol_type = other DNA
gtagattcacacct

SEQ ID NO: 341 - DNA, 14 bp, synthetic construct
  source 1..14: mol_type = other DNA
ggcaaggccatcac

SEQ ID NO: 342 - DNA, 14 bp, synthetic construct
  source 1..14: mol_type = other DNA
cggagggtcgccaa

SEQ ID NO: 343 - DNA, 14 bp, synthetic construct
  source 1..14: mol_type = other DNA
tcggggactgtcac

SEQ ID NO: 344 - DNA, 14 bp, synthetic construct
  source 1..14: mol_type = other DNA
gcctcgtggctgaa

SEQ ID NO: 345 - DNA, 14 bp, synthetic construct
  source 1..14: mol_type = other DNA
ggcatgaacctgga

SEQ ID NO: 346 - DNA, 14 bp, synthetic construct
  source 1..14: mol_type = other DNA
gcaggggatcgggg

SEQ ID NO: 347 - DNA, 14 bp, synthetic construct
  source 1..14: mol_type = other DNA
ggacgaccctgaca

SEQ ID NO: 348 - DNA, 14 bp, synthetic construct
  source 1..14: mol_type = other DNA
cgcggccttaaaaa

SEQ ID NO: 349 - DNA, 14 bp, synthetic construct
  source 1..14: mol_type = other DNA
agcggctccagaag

SEQ ID NO: 350 - DNA, 14 bp, synthetic construct
  source 1..14: mol_type = other DNA
cagcttggctcgcg

SEQ ID NO: 351 - DNA, 14 bp, synthetic construct
  source 1..14: mol_type = other DNA
ccgccgcactcccg

SEQ ID NO: 352 - DNA, 14 bp, synthetic construct
  source 1..14: mol_type = other DNA
ggtacccgggtgag

SEQ ID NO: 353 - DNA, 14 bp, synthetic construct
  source 1..14: mol_type = other DNA
cagtccagttgtgt

SEQ ID NO: 354 - DNA, 14 bp, synthetic construct
  source 1..14: mol_type = other DNA
gcgcgtcaaagtgc

SEQ ID NO: 355 - DNA, 14 bp, synthetic construct
  source 1..14: mol_type = other DNA
gtttggagccacaa

SEQ ID NO: 356 - DNA, 14 bp, synthetic construct
  source 1..14: mol_type = other DNA
gccgcgtagaagga

SEQ ID NO: 357 - DNA, 14 bp, synthetic construct
  source 1..14: mol_type = other DNA
gcgggactgacttc

SEQ ID NO: 358 - DNA, 14 bp, synthetic construct
  source 1..14: mol_type = other DNA
ccgtggccccggcg

SEQ ID NO: 359 - DNA, 14 bp, synthetic construct
  source 1..14: mol_type = other DNA
aggagcagtttcga

SEQ ID NO: 360 - DNA, 14 bp, synthetic construct
  source 1..14: mol_type = other DNA
gggtttatctcagg

SEQ ID NO: 361 - DNA, 14 bp, synthetic construct
  source 1..14: mol_type = other DNA
ctggacaacagtca

SEQ ID NO: 362 - DNA, 14 bp, synthetic construct
  source 1..14: mol_type = other DNA
gcagccttctgatt

SEQ ID NO: 363 - DNA, 14 bp, synthetic construct
  source 1..14: mol_type = other DNA
ccgtcaagtgtaga

SEQ ID NO: 364 - DNA, 14 bp, synthetic construct
  source 1..14: mol_type = other DNA
agtgtcgggtatca

SEQ ID NO: 365 - DNA, 14 bp, synthetic construct
  source 1..14: mol_type = other DNA
ctctggaggaaatc

SEQ ID NO: 366 - DNA, 14 bp, synthetic construct
  source 1..14: mol_type = other DNA
agggccgcgctgga

SEQ ID NO: 367 - DNA, 14 bp, synthetic construct
  source 1..14: mol_type = other DNA
ccaaagaaacttgg

SEQ ID NO: 368 - DNA, 14 bp, synthetic construct
  source 1..14: mol_type = other DNA
gaagcgacgcaagt

SEQ ID NO: 369 - DNA, 14 bp, synthetic construct
  source 1..14: mol_type = other DNA
agcgtgcctttcgg

SEQ ID NO: 370 - DNA, 14 bp, synthetic construct
  source 1..14: mol_type = other DNA
ggctgcccgctgga

SEQ ID NO: 371 - DNA, 14 bp, synthetic construct
  source 1..14: mol_type = other DNA
aagactcgtcctac

SEQ ID NO: 372 - DNA, 14 bp, synthetic construct
  source 1..14: mol_type = other DNA
gaccttcccgagag

SEQ ID NO: 373 - DNA, 14 bp, synthetic construct
  source 1..14: mol_type = other DNA
gccgagggcagaga

SEQ ID NO: 374 - DNA, 14 bp, synthetic construct
  source 1..14: mol_type = other DNA
cttggcttcttaaa

SEQ ID NO: 375 - DNA, 14 bp, synthetic construct
  source 1..14: mol_type = other DNA
caacgggccggaaa

SEQ ID NO: 376 - DNA, 14 bp, synthetic construct
  source 1..14: mol_type = other DNA
agtcctgccagcgc

SEQ ID NO: 377 - DNA, 14 bp, synthetic construct
  source 1..14: mol_type = other DNA
ccggaggaaaccag

SEQ ID NO: 378 - DNA, 14 bp, synthetic construct
  source 1..14: mol_type = other DNA
cggggtccctacag

SEQ ID NO: 379 - DNA, 14 bp, synthetic construct
  source 1..14: mol_type = other DNA
cgcgcgcgaatgcg

SEQ ID NO: 380 - DNA, 14 bp, synthetic construct
  source 1..14: mol_type = other DNA
agaaaactgcgtcc

SEQ ID NO: 381 - DNA, 14 bp, synthetic construct
  source 1..14: mol_type = other DNA
cttccgagcggcct

SEQ ID NO: 382 - DNA, 14 bp, synthetic construct
  source 1..14: mol_type = other DNA
ggtttgggattgtg

SEQ ID NO: 383 - DNA, 14 bp, synthetic construct
  source 1..14: mol_type = other DNA
caaaggtgggctcg

SEQ ID NO: 384 - DNA, 14 bp, synthetic construct
  source 1..14: mol_type = other DNA
aagcgattattccc

SEQ ID NO: 385 - DNA, 14 bp, synthetic construct
  source 1..14: mol_type = other DNA
atccacatccccga

SEQ ID NO: 386 - DNA, 14 bp, synthetic construct
  source 1..14: mol_type = other DNA
gccatgtaaggcac

SEQ ID NO: 387 - DNA, 14 bp, synthetic construct
  source 1..14: mol_type = other DNA
gcgccgaatgggag

SEQ ID NO: 388 - DNA, 14 bp, synthetic construct
  source 1..14: mol_type = other DNA
ccgcgacagtgagt

SEQ ID NO: 389 - DNA, 14 bp, synthetic construct
  source 1..14: mol_type = other DNA
accgcggaggcggc

SEQ ID NO: 390 - DNA, 14 bp, synthetic construct
  source 1..14: mol_type = other DNA
ggaggggcgcttat

SEQ ID NO: 391 - DNA, 14 bp, synthetic construct
  source 1..14: mol_type = other DNA
ctgagtataaaagc

SEQ ID NO: 392 - DNA, 14 bp, synthetic construct
  source 1..14: mol_type = other DNA
ttggcagcaaattg

SEQ ID NO: 393 - DNA, 14 bp, synthetic construct
  source 1..14: mol_type = other DNA
tgcgagggtctgga

SEQ ID NO: 394 - DNA, 14 bp, synthetic construct
  source 1..14: mol_type = other DNA
cgtagcgcggcgct

SEQ ID NO: 395 - DNA, 14 bp, synthetic construct
  source 1..14: mol_type = other DNA
cgccgccgcggagg

SEQ ID NO: 396 - DNA, 14 bp, synthetic construct
  source 1..14: mol_type = other DNA
ccgagcgcccgcga

SEQ ID NO: 397 - DNA, 14 bp, synthetic construct
  source 1..14: mol_type = other DNA
cggccattggcggc

SEQ ID NO: 398 - DNA, 14 bp, synthetic construct
  source 1..14: mol_type = other DNA
gttccattgacgca

SEQ ID NO: 399 - DNA, 14 bp, synthetic construct
  source 1..14: mol_type = other DNA
ctttggccatacaa

SEQ ID NO: 400 - DNA, 14 bp, synthetic construct
  source 1..14: mol_type = other DNA
aataaccagcggga

SEQ ID NO: 401 - DNA, 14 bp, synthetic construct
  source 1..14: mol_type = other DNA
tgcggggagcctag

SEQ ID NO: 402 - DNA, 14 bp, synthetic construct
  source 1..14: mol_type = other DNA
gggcgccgccctag

SEQ ID NO: 403 - DNA, 14 bp, synthetic construct
  source 1..14: mol_type = other DNA
tctgcgctgtgcgc

SEQ ID NO: 404 - DNA, 14 bp, synthetic construct
  source 1..14: mol_type = other DNA
ctgccccttcccgt

SEQ ID NO: 405 - DNA, 14 bp, synthetic construct
  source 1..14: mol_type = other DNA
ccggagcgaggaaa

SEQ ID NO: 406 - DNA, 14 bp, synthetic construct
  source 1..14: mol_type = other DNA
gggaacctcgtctg

SEQ ID NO: 407 - DNA, 14 bp, synthetic construct
  source 1..14: mol_type = other DNA
tgagagccgaagca

SEQ ID NO: 408 - DNA, 14 bp, synthetic construct
  source 1..14: mol_type = other DNA
gcagtgcgtgcgag

SEQ ID NO: 409 - DNA, 14 bp, synthetic construct
  source 1..14: mol_type = other DNA
aggaagggccgtcc

SEQ ID NO: 410 - DNA, 14 bp, synthetic construct
  source 1..14: mol_type = other DNA
acgccccctacgat

SEQ ID NO: 411 - DNA, 14 bp, synthetic construct
  source 1..14: mol_type = other DNA
cagccaatcgtagg

SEQ ID NO: 412 - DNA, 14 bp, synthetic construct
  source 1..14: mol_type = other DNA
gtgagtaggatgac

SEQ ID NO: 413 - DNA, 14 bp, synthetic construct
  source 1..14: mol_type = other DNA
aggtcctcgggggt

SEQ ID NO: 414 - DNA, 14 bp, synthetic construct
  source 1..14: mol_type = other DNA
caggtgacaggcat

SEQ ID NO: 415 - DNA, 14 bp, synthetic construct
  source 1..14: mol_type = other DNA
acagcacccaccac

SEQ ID NO: 416 - DNA, 14 bp, synthetic construct
  source 1..14: mol_type = other DNA
aggagaaacaggac

SEQ ID NO: 417 - DNA, 14 bp, synthetic construct
  source 1..14: mol_type = other DNA
aaggggtccgcacg

SEQ ID NO: 418 - DNA, 14 bp, synthetic construct
  source 1..14: mol_type = other DNA
cgtacagtagcgcg

SEQ ID NO: 419 - DNA, 14 bp, synthetic construct
  source 1..14: mol_type = other DNA
gtagactttgcaag

SEQ ID NO: 420 - DNA, 14 bp, synthetic construct
  source 1..14: mol_type = other DNA
ggggttagtacccc

SEQ ID NO: 421 - DNA, 14 bp, synthetic construct
  source 1..14: mol_type = other DNA
ggtgcccgcgatgg

SEQ ID NO: 422 - DNA, 14 bp, synthetic construct
  source 1..14: mol_type = other DNA
gcgccccagcgtca

SEQ ID NO: 423 - DNA, 14 bp, synthetic construct
  source 1..14: mol_type = other DNA
cgcccctccccgtt

SEQ ID NO: 424 - DNA, 14 bp, synthetic construct
  source 1..14: mol_type = other DNA
gagaagcctgctcg

SEQ ID NO: 425 - DNA, 14 bp, synthetic construct
  source 1..14: mol_type = other DNA
ggttgggttgacac

SEQ ID NO: 426 - DNA, 14 bp, synthetic construct
  source 1..14: mol_type = other DNA
gcgcgggagctggt

SEQ ID NO: 427 - DNA, 14 bp, synthetic construct
  source 1..14: mol_type = other DNA
ttcttgcgcgcccg

SEQ ID NO: 428 - DNA, 14 bp, synthetic construct
  source 1..14: mol_type = other DNA
agggcgctgtgcga

SEQ ID NO: 429 - DNA, 14 bp, synthetic construct
  source 1..14: mol_type = other DNA
tggggattaaaacc

SEQ ID NO: 430 - DNA, 14 bp, synthetic construct
  source 1..14: mol_type = other DNA
gaggaaggcggcgt

SEQ ID NO: 431 - DNA, 14 bp, synthetic construct
  source 1..14: mol_type = other DNA
cgtcgagcgaagcg

SEQ ID NO: 432 - DNA, 14 bp, synthetic construct
  source 1..14: mol_type = other DNA
cgcggaggtgacaa

SEQ ID NO: 433 - DNA, 14 bp, synthetic construct
  source 1..14: mol_type = other DNA
actcctccgaggcg

SEQ ID NO: 434 - DNA, 14 bp, synthetic construct
  source 1..14: mol_type = other DNA
ggcgcacctgctcg

SEQ ID NO: 435 - DNA, 14 bp, synthetic construct
  source 1..14: mol_type = other DNA
ttaggcccacgccc

SEQ ID NO: 436 - DNA, 14 bp, synthetic construct
  source 1..14: mol_type = other DNA
gtagccttgggcgc

SEQ ID NO: 437 - DNA, 14 bp, synthetic construct
  source 1..14: mol_type = other DNA
gcgtcctctcccac

SEQ ID NO: 438 - DNA, 14 bp, synthetic construct
  source 1..14: mol_type = other DNA
cccgcagtgtcact

SEQ ID NO: 439 - DNA, 14 bp, synthetic construct
  source 1..14: mol_type = other DNA
gattattattgcat

SEQ ID NO: 440 - DNA, 14 bp, synthetic construct
  source 1..14: mol_type = other DNA
gataaagaggcgcg

SEQ ID NO: 441 - DNA, 14 bp, synthetic construct
  source 1..14: mol_type = other DNA
gggttccaagccaa

SEQ ID NO: 442 - DNA, 14 bp, synthetic construct
  source 1..14: mol_type = other DNA
acacacacagcaaa

SEQ ID NO: 443 - DNA, 14 bp, synthetic construct
  source 1..14: mol_type = other DNA
tggagcatcggtga

SEQ ID NO: 444 - DNA, 14 bp, synthetic construct
  source 1..14: mol_type = other DNA
gataatttcgttgt

SEQ ID NO: 445 - DNA, 14 bp, synthetic construct
  source 1..14: mol_type = other DNA
aatcgtgatggtgt

SEQ ID NO: 446 - DNA, 14 bp, synthetic construct
  source 1..14: mol_type = other DNA
ggacgtatttatac

SEQ ID NO: 447 - DNA, 14 bp, synthetic construct
  source 1..14: mol_type = other DNA
gagatagtcccctt

SEQ ID NO: 448 - DNA, 14 bp, synthetic construct
  source 1..14: mol_type = other DNA
agggatgactttgg

SEQ ID NO: 449 - DNA, 14 bp, synthetic construct
  source 1..14: mol_type = other DNA
cggttggtccgcgc

SEQ ID NO: 450 - DNA, 14 bp, synthetic construct
  source 1..14: mol_type = other DNA
ccagcgcacgggca

SEQ ID NO: 451 - DNA, 14 bp, synthetic construct
  source 1..14: mol_type = other DNA
ggaaccactgcgac

SEQ ID NO: 452 - DNA, 14 bp, synthetic construct
  source 1..14: mol_type = other DNA
gcccagacgcactc

SEQ ID NO: 453 - DNA, 14 bp, synthetic construct
  source 1..14: mol_type = other DNA
caaagcgagtttcc

SEQ ID NO: 454 - DNA, 14 bp, synthetic construct
  source 1..14: mol_type = other DNA
tagggagacgagaa

SEQ ID NO: 455 - DNA, 14 bp, synthetic construct
  source 1..14: mol_type = other DNA
gtccgtgttgggaa

SEQ ID NO: 456 - DNA, 14 bp, synthetic construct
  source 1..14: mol_type = other DNA
ctggaattgggttc

SEQ ID NO: 457 - DNA, 14 bp, synthetic construct
  source 1..14: mol_type = other DNA
ttacagcgcgtcgg

SEQ ID NO: 458 - DNA, 14 bp, synthetic construct
  source 1..14: mol_type = other DNA
gaggcgcttatcgg

SEQ ID NO: 459 - DNA, 14 bp, synthetic construct
  source 1..14: mol_type = other DNA
tcgaaaggaaccga

SEQ ID NO: 460 - DNA, 14 bp, synthetic construct
  source 1..14: mol_type = other DNA
gcgtacccccgtag

SEQ ID NO: 461 - DNA, 14 bp, synthetic construct
  source 1..14: mol_type = other DNA
tctctccaccatgg

SEQ ID NO: 462 - DNA, 14 bp, synthetic construct
  source 1..14: mol_type = other DNA
ctgaatttccccga

SEQ ID NO: 463 - DNA, 14 bp, synthetic construct
  source 1..14: mol_type = other DNA
gcgcggcagctctc

SEQ ID NO: 464 - DNA, 14 bp, synthetic construct
  source 1..14: mol_type = other DNA
cctaggcggcggag

SEQ ID NO: 465 - DNA, 14 bp, synthetic construct
  source 1..14: mol_type = other DNA
ggcgcggagggatc

SEQ ID NO: 466 - DNA, 14 bp, synthetic construct
  source 1..14: mol_type = other DNA
gaggagccgcaccg

SEQ ID NO: 467 - DNA, 14 bp, synthetic construct
  source 1..14: mol_type = other DNA
agtccttgcattgg

SEQ ID NO: 468 - DNA, 14 bp, synthetic construct
  source 1..14: mol_type = other DNA
aggtgtagcaagct

SEQ ID NO: 469 - DNA, 14 bp, synthetic construct
  source 1..14: mol_type = other DNA
cctccaccaatgca

SEQ ID NO: 470 - DNA, 14 bp, synthetic construct
  source 1..14: mol_type = other DNA
tcacagagcacaga

SEQ ID NO: 471 - DNA, 14 bp, synthetic construct
  source 1..14: mol_type = other DNA
gggacggtagggca

SEQ ID NO: 472 - DNA, 14 bp, synthetic construct
  source 1..14: mol_type = other DNA
ctcccctccacaga

SEQ ID NO: 473 - DNA, 14 bp, synthetic construct
  source 1..14: mol_type = other DNA
caggctgccatctg

SEQ ID NO: 474 - DNA, 14 bp, synthetic construct
  source 1..14: mol_type = other DNA
gcgggggctcgcca

SEQ ID NO: 475 - DNA, 14 bp, synthetic construct
  source 1..14: mol_type = other DNA
ccgcgaaagtctga

SEQ ID NO: 476 - DNA, 14 bp, synthetic construct
  source 1..14: mol_type = other DNA
ggagggatccgcgt

SEQ ID NO: 477 - DNA, 14 bp, synthetic construct
  source 1..14: mol_type = other DNA
gcggggagaaatca

SEQ ID NO: 478 - DNA, 14 bp, synthetic construct
  source 1..14: mol_type = other DNA
cgagggtgctcgag

SEQ ID NO: 479 - DNA, 14 bp, synthetic construct
  source 1..14: mol_type = other DNA
gggacgcgcccact

SEQ ID NO: 480 - DNA, 14 bp, synthetic construct
  source 1..14: mol_type = other DNA
cctgagagagacgt

SEQ ID NO: 481 - DNA, 14 bp, synthetic construct
  source 1..14: mol_type = other DNA
cgcgccgcggcttg

SEQ ID NO: 482 - DNA, 14 bp, synthetic construct
  source 1..14: mol_type = other DNA
cggcacacgcgctc

SEQ ID NO: 483 - DNA, 14 bp, synthetic construct
  source 1..14: mol_type = other DNA
gcgggaccaagagg

SEQ ID NO: 484 - DNA, 14 bp, synthetic construct
  source 1..14: mol_type = other DNA
atccagaagggaca

SEQ ID NO: 485 - DNA, 14 bp, synthetic construct
  source 1..14: mol_type = other DNA
acaatagcgcgcgc

SEQ ID NO: 486 - DNA, 14 bp, synthetic construct
  source 1..14: mol_type = other DNA
gcgcgcccccctca

SEQ ID NO: 487 - DNA, 14 bp, synthetic construct
  source 1..14: mol_type = other DNA
gaggggtgccgttc

SEQ ID NO: 488 - DNA, 14 bp, synthetic construct
  source 1..14: mol_type = other DNA
cgcgggcagtgcct

SEQ ID NO: 489 - DNA, 14 bp, synthetic construct
  source 1..14: mol_type = other DNA
gagccgcgtgtgga

SEQ ID NO: 490 - DNA, 14 bp, synthetic construct
  source 1..14: mol_type = other DNA
cgggcgcgtgtctc

SEQ ID NO: 491 - DNA, 14 bp, synthetic construct
  source 1..14: mol_type = other DNA
gccgggtagctcgg

SEQ ID NO: 492 - DNA, 14 bp, synthetic construct
  source 1..14: mol_type = other DNA
gcgggatcccaagg

SEQ ID NO: 493 - DNA, 14 bp, synthetic construct
  source 1..14: mol_type = other DNA
cactacccggaggg

SEQ ID NO: 494 - DNA, 14 bp, synthetic construct
  source 1..14: mol_type = other DNA
ggggagcgcgtgtc

SEQ ID NO: 495 - DNA, 14 bp, synthetic construct
  source 1..14: mol_type = other DNA
gcggcaggggaaag

SEQ ID NO: 496 - DNA, 14 bp, synthetic construct
  source 1..14: mol_type = other DNA
ggcgaggccgtcgg

SEQ ID NO: 497 - DNA, 14 bp, synthetic construct
  source 1..14: mol_type = other DNA
tatgggacctcgga

SEQ ID NO: 498 - DNA, 14 bp, synthetic construct
  source 1..14: mol_type = other DNA
ggcaagttcagtcc

SEQ ID NO: 499 - DNA, 14 bp, synthetic construct
  source 1..14: mol_type = other DNA
aaactgggcaccgt

SEQ ID NO: 500 - DNA, 14 bp, synthetic construct
  source 1..14: mol_type = other DNA
agggagagtgagag

SEQ ID NO: 501 - DNA, 14 bp, synthetic construct
  source 1..14: mol_type = other DNA
tccctaccaatagc

SEQ ID NO: 502 - DNA, 14 bp, synthetic construct
  source 1..14: mol_type = other DNA
ctaacgtgataaca

SEQ ID NO: 503 - DNA, 14 bp, synthetic construct
  source 1..14: mol_type = other DNA
gattgagccattga

SEQ ID NO: 504 - DNA, 14 bp, synthetic construct
  source 1..14: mol_type = other DNA
tgcatgagcatctg

SEQ ID NO: 505 - DNA, 14 bp, synthetic construct
  source 1..14: mol_type = other DNA
gcgcagtaaccagg

SEQ ID NO: 506 - DNA, 14 bp, synthetic construct
  source 1..14: mol_type = other DNA
tcagaatccttaat

SEQ ID NO: 507 - DNA, 14 bp, synthetic construct
  source 1..14: mol_type = other DNA
cgaagcccggagcg

SEQ ID NO: 508 - DNA, 14 bp, synthetic construct
  source 1..14: mol_type = other DNA
gccgaagtactgta

SEQ ID NO: 509 - DNA, 14 bp, synthetic construct
  source 1..14: mol_type = other DNA
agaggggccacgga

SEQ ID NO: 510 - DNA, 14 bp, synthetic construct
  source 1..14: mol_type = other DNA
ccggctgcaacttc

SEQ ID NO: 511 - DNA, 14 bp, synthetic construct
  source 1..14: mol_type = other DNA
ttataattggtgtg

SEQ ID NO: 512 - DNA, 14 bp, synthetic construct
  source 1..14: mol_type = other DNA
attgggcggaggga

SEQ ID NO: 513 - DNA, 14 bp, synthetic construct
  source 1..14: mol_type = other DNA
ccctgtccgctcat

SEQ ID NO: 514 - DNA, 14 bp, synthetic construct
  source 1..14: mol_type = other DNA
cgctcgctgctggg

SEQ ID NO: 515 - DNA, 14 bp, synthetic construct
  source 1..14: mol_type = other DNA
ctggtcacctagtg

SEQ ID NO: 516 - DNA, 14 bp, synthetic construct
  source 1..14: mol_type = other DNA
gcttccaccacact

SEQ ID NO: 517 - DNA, 14 bp, synthetic construct
  source 1..14: mol_type = other DNA
atcaaaaagatgac

SEQ ID NO: 518 - DNA, 14 bp, synthetic construct
  source 1..14: mol_type = other DNA
agcctgtatgaggg